(19) United States
(12) Patent Application Publication
(10) Pub. No.: US 2023/0203587 A1
(43) Pub. Date: Jun. 29, 2023

(54) USE OF MICROVESICLE SIGNATURE FOR THE DIAGNOSIS AND TREATMENT OF KIDNEY TRANSPLANT REJECTION

(71) Applicants: Exosome Diagnostics, Inc., Waltham, MA (US); The Brigham and Women's Hospital, Inc., Boston, MA (US)

(72) Inventors: Johan SKOG, Lincoln, MA (US); Vasisht TADIGOTLA, Newton, MA (US); James HURLEY, Marblehead, MA (US); Jamil AZZI, Boston, MA (US)

(21) Appl. No.: 17/928,452

(22) PCT Filed: May 28, 2021
(86) PCT No.: PCT/US2021/034857

§ 371 Date: Nov. 29, 2022

Related U.S. Application Data
(60) Provisional application No. 63/032,267, filed on May 29, 2020.

(51) Int. Cl. C12Q 1/6883; C12Q 1/686

(52) U.S. Cl. CPC C12Q 1/6883; C12Q 1/686; C12Q 2600/118; C12Q 2600/158

(57) ABSTRACT

The present disclosure relates to methods of identifying and treating kidney rejection in a subject comprising analyzing microvesicular RNA, cell-free DNA or the combination of microvesicular and cell-free DNA.”

### RELATED APPLICATIONS

This application claims priority to, and the benefit of, U.S. Provisional Application No. 63/054,971, filed May 29, 2020, the contents of which is incorporated herein by reference in its entirety.

### GOVERNMENT SUPPORT

This invention was made with government support under Grant No. RO1-AI134842 awarded by the National Institutes of Health and under Grant No. F32D111106 awarded by the National Institutes of Health, The government has certain rights in the invention.

### BACKGROUND

In 2018 in the United States alone, there were an estimated 21,167 kidney transplants. Although the introduction of more potent immunosuppressive drugs has decreased the incidence of acute rejection following transplantation, roughly 10% of kidney transplant patients will experience acute rejection within the first year. Moreover, episodes of acute rejection, especially those that occur within the first year, are associated with poor long-term allograft outcome. The gold standard in the diagnoses of acute rejection following kidney rejection is kidney allograft biopsies followed by histopathological evaluation. However, such biopsies suffer from several limitations, including invasiveness, cost and inter-observer variability. Indeed, repeated biopsies for the monitoring of rejection has been associated with increased negative complications, on top of the already increased cost. Attempts at developing an alternative to biopsies for the diagnosis of kidney transplant rejection have thus far failed. Serum creatinine (SCr) and urinary protein excretion are traditional biomarkers currently used to monitor the kidney graft function, but they lack sensitivity, specificity and predictive ability, Therefore, there is an urgent need of an accurate, non-invasive method of identifying kidney transplant rejection, particularly at an early stage following transplant.

### SUMMARY

The present disclosure provides methods of identifying kidney transplant rejection in a subject who has undergone a kidney transplant. The present disclosure provides methods of treating a kidney transplant rejection in a subject who has undergone a kidney transplant. The present disclosure provides methods of determining the risk of a kidney transplant rejection in a subject who has undergone a kidney transplant. In some aspects, the kidney transplant rejection is any-cause rejection.

The present disclosure provides methods of identifying antibody-mediated kidney transplant rejection in a subject who has undergone a kidney transplant rejection. The present disclosure provides methods of treating antibody-mediated kidney transplant rejection in a subject who has undergone a kidney transplant rejection. The present disclosure provides methods of determining the risk of an antibody-mediated kidney transplant rejection in a subject who has undergone a kidney transplant rejection.

The present disclosure provides methods of identifying cell-mediated kidney transplant rejection in a subject who has undergone a kidney transplant rejection. The present disclosure provides methods of treating cell-mediated kidney transplant rejection in a subject who has undergone a kidney transplant rejection. The present disclosure provides methods of determining the risk of a cell-mediated kidney transplant rejection in a subject who has undergone a kidney transplant rejection. In some aspects, the cell-mediated kidney transplant rejection is T-cell mediated kidney transplant rejection.

The present disclosure provides methods of identifying antibody-mediated kidney transplant rejection or cell-mediated kidney transplant rejection in a subject who has undergone a kidney transplant and has been identified as having a kidney transplant rejection. The present disclosure provides methods of determining the risk of an antibody-mediated kidney transplant rejection as opposed to a cell-mediated kidney transplant rejection in a subject who has undergone a kidney transplant and has been identified as having a kidney transplant rejection. In some aspects, the cell-mediated kidney transplant rejection is T-cell mediated kidney transplant rejection.

The present disclosure provides methods of determining the risk of a kidney transplant rejection in a subject who has undergone a kidney transplant, the method comprising: a) determining the expression level of at least two of 15 biomarkers in microvesicular RNA isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP; b) inputting the expression levels from step (a) into an algorithm to generate a score; c) determining the risk of a kidney transplant rejection in the subject based on the score.

In some aspects, a kidney transplant rejection can be any-cause kidney transplant rejection.

In some aspects of the preceding methods, step (a) can comprise determining the expression level of: a.) at least three of the 15 biomarkers; h) at least four of the 15 biomarkers; c) at least five of the 15 biomarkers; d) at least six of the 15 biomarkers; e) at least seven of the 15 biomarkers; f) at least eight of the 15 biomarkers; g) at least nine of the 15 biomarkers; h) at least ten of the 15 biomarkers; i) at least 11 of the 15 biomarkers; j) at least 12 of the 15 biomarkers, k) at least 13 of the 15 biomarkers; or 1) at least 14 of the 15 biomarkers. In some aspects of the preceding methods, step (a) can comprise determining the expression level of each of the 15 biomarkers.

In some aspects of the preceding methods, determining the risk of a kidney transplant rejection in the subject based on the score can comprise: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

The present disclosure provides methods of determining the risk of an antibody-mediated kidney transplant rejection as opposed to a cell-mediated kidney transplant rejection in a subject who has undergone a kidney transplant and has been identified as having a kidney transplant rejection and/or identified as being at high risk of having a kidney transplant rejection, the method comprising: a) determining the expression level of at least two of five biomarkers in microvesicular RNA isolated from a biological sample from the subject, wherein the five biomarkers comprise CD74, C3, CXCL11, CD44 and IFNAR2; b) inputting the expression levels from step (a) into an algorithm to generate a score; and c) determining the risk of an antibody-mediated kidney transplant rejection as opposed to a cell-mediated kidney transplant rejection in the subject based on the score.

In some aspects of the preceding methods, step (a) can comprise determining the expression level of: a) at least three of the five biomarkers; or b) at least four of the five biomarkers. In some aspects of the preceding methods, step (a) can comprise determining the expression level of each of the five biomarkers.

In some aspects of the preceding methods, determining the risk of an antibody-mediated kidney transplant rejection as opposed to a cell-mediated kidney transplant rejection in the subject based on the score can comprise: i) comparing the score to a predetermined cutoff value; and ii) determining that the at the subject is at a higher risk of having an antibody-mediated kidney transplant rejection as opposed to a cell-mediated kidney transplant rejection when the score is greater than or equal to the predetermined cutoff value, or determining that the subject is at a higher risk of having a cell-mediated kidney transplant rejection when the score is less than the predetermined cutoff value.

The present disclosure provides methods of determining the risk of a cell-mediated kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CD74, CXCL11, C3, CCL2, B2M, IL15, IL18BP, FPR2, ALOX5AP, IL1RAP, TLR1, NAMPT and IL1R2; b) inputting the expression levels from step (a) into an algorithm to generate a score; c) determining the risk of a cell-mediated kidney transplant rejection in the subject based on the score.

In some aspects of the preceding methods, determining the risk of a cell-mediated kidney transplant rejection in the subject based on the score can comprise: i) comparing the score to a predetermined cutoff value; and ii) determining that the at the subject is at a high risk of having a cell-mediated kidney transplant rejection when the score is greater than or equal to the predetermined cutoff value or determining that the subject is at low risk of having a cell-mediated kidney transplant rejection when the score is less than the predetermined cutoff value.

The present disclosure provides methods of determining the risk of an antibody-mediated kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1; b) inputting the expression levels from step (a) into an algorithm to generate a score; and c) determining the risk of an antibody-mediated kidney transplant rejection in the subject based on the score.

In some aspects of the preceding methods, determining the risk of an antibody-mediated kidney transplant rejection in the subject based on the score can comprise: i) comparing the score to a predetermined cutoff value; and ii) determining that the at the subject is at a high risk of having an antibody-mediated kidney transplant rejection when the score is greater than or equal to the predetermined cutoff value or determining that the subject is at low risk of having an antibody-mediated kidney transplant rejection when the score is less than the predetermined cutoff value.

In some aspects of the preceding methods, step (a) can comprise determining the expression level of: a) at least three of the 13 biomarkers; b) at least four of the 13 biomarkers; c) at least five of the 13 biomarkers; d) at least six of the 13 biomarkers; e) at least seven of the 13 biomarkers; 0 at least eight of the 13 biomarkers; g) at least nine of the 13 biomarkers; h) at least ten of the 13 biomarkers; i) at least 11 of the 13 biomarkers; j) at least 12 of the 13 biomarkers. In some aspects of the preceding methods, step (a) can comprise determining the expression level of each of the 13 biomarkers.

In some aspects of the preceding methods, a biological sample can be a urine sample, preferably wherein the urine sample is: a) a first-catch urine sample; or b) a second voided urine sample.

In some aspects of the preceding methods, a biological sample can have a volume of between at least about 1 ml to at least about 50 ml, preferably wherein the biological sample has a volume of at least about 3 ml, preferably wherein the biological sample has a volume of up to about 20 ml.

In some aspects of the preceding methods, step (a) can further comprise: (i) determining the expression level of at least one reference biomarker; (ii) normalizing the expression level of the at least two biomarkers to the expression level of the at least one reference biomarker, and wherein inputting the expression levels from step (a) into an algorithm to generate a score comprises inputting the normalized expression levels from step (a) into an algorithm to generate a score.

In some aspects of the preceding methods, an at least one reference biomarker can comprise PGK1.

In some aspects of the preceding methods, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis or any combination thereof

In some aspects of the preceding methods, an algorithm can be the product of a feature selection wrapper algorithm, a machine learning algorithm, a trained classifier built from at least one predictive classification algorithm or any combination thereof, preferably wherein the predictive classification algorithm, the feature selection wrapper algorithm, and/or the machine learning algorithm comprises XGBoost (XGB), random forest (RF), Lasso and Elastic-Net Regularized. Generalized Linear Models (glmnet), cforest, classification and regression tree (CART), treebag, k nearest-neighbor (knn), neural network (nnet), support vector machine-radial (SVM-radial), support vector machine-linear (SVM-linear), naïve bayes (NB), multilayer perceptron (rasp), Boruta or any combination thereof.

In some aspects of the preceding methods, a predetermined cutoff value can have: i) a negative predictive value of at least about 80%; ii) a positive predictive value of at least about 80%; iii) a sensitivity of at least about 80%; iv) a specificity of at least about 80%; or v) any combination thereof.

In some aspects of the preceding methods, the methods can further comprise administering to a subject identified as being at risk for a kidney transplant rejection at least one kidney transplant rejection therapy, preferably wherein the at least one kidney transplant rejection therapy comprises administering to the subject at least one therapeutically effective amount of at least one immunosuppressant, at least one steroid, at least one corticosteroid, at least one anti-T-cell antibody, mycophenolate mofetil (MMF), cyclosporine A (CsA), tacrolimus, azathioprine, muromonab (OKT-3), anti-thymocyte (ATG), anti-lymphocyte globulin (ALG), Campath (alemtuzumab), prednisone, mycophenolic acid, rapamycin, belatacept, intravenous immunoglobulin (IVIg), an anti-CD20 agent, rituximab, bortezomib, or any combination thereof.

In some aspects of the preceding methods, the methods can further comprise administering to a subject identified as being at risk for a cell-mediated kidney transplant rejection at least one cell-mediated kidney transplant rejection therapy, preferably wherein the at least one cell-mediated kidney transplant rejection therapy comprises administering to the subject at least one therapeutically effective amount of at least one steroid, at least one corticosteroid, muromonab (OKT-3), anti-thymocyte globulin (ATG), Campath (alemtuzumab), prednisone, tacrolimus cyclosporine A, mycophenolic acid, azathioprine, rapamycin, amount of belatacept, or any combination thereof

In some aspects of the preceding methods, the methods can further comprise administering to a subject identified as being at risk for an antibody-mediated kidney transplant rejection at least one antibody-mediated kidney transplant rejection therapy, preferably wherein the at least one antibody-mediated kidney transplant rejection therapy comprises administering to the subject at least one therapeutically effective amount of at least one steroid, at least one corticosteroid, anti-thymocyte globulin (ATG), intravenous immunoglobulin (IVIg), an anti-CD20 agent, rituximab, bortezomib, or any combination thereof.

In some aspects of the preceding methods, a subject has not undergone a renal biopsy.

Any of the aspects described herein can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%. 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1A is a graph showing area under the curve-receiver operating characteristics analysis for the 8-gene signature of the present disclosure in the training set.

FIG. 1B is a graph showing area under the curve-receiver operating characteristics analysis for the 8-gene signature of the present disclosure in the validation set.

FIG. 2A is a graph showing the probability of any-cause rejection based on the 8-gene signature of the present disclosure in the training set.

FIG. 2B is a graph showing the probability of any-cause rejection based on the 8-gene signature of the present disclosure in the validation set.

FIG. 3A is a graph showing area under the curve-receiver operating characteristics analysis for the 13-gene signature of the present disclosure in the training set.

FIG. 3B is a graph showing area under the curve-receiver operating characteristics analysis for the 13-gene signatures of the present disclosure in the validation set.

FIG. 4A is a graph showing the probability of any-cause rejection based on the 13-gene signature of the present disclosure in the training set.

FIG. 4B is a graph showing the probability of any-cause rejection based on the 13-gene signature of the present disclosure in the validation set.

FIG. 5A is a graph showing area under the curve-receiver operating characteristics analysis for the 10-gene signature of the present disclosure in the training set.

FIG. SB is a graph showing area under the curve-receiver operating characteristics analysis for the 10-gene signature of the present disclosure in the validation set.

FIG. 6A is a graph showing the probability of any-cause rejection based on the 10-gene signature of the present disclosure in the training set.

FIG. 6B is a graph showing the probability of any-cause rejection based on the 10-gene signature of the present disclosure in the validation set.

FIG. 7A is a graph showing area under the curve-receiver operating characteristics analysis for the 5-gene signature (F3, CD74, CXCL10, UBE2D2 and IFNA4) of the present disclosure in the training set.

FIG. 7B is a graph showing area under the curve-receiver operating characteristics analysis for the 5-gene signature (F3, CD74, CXCL10, UBE2D2 and IFNA4) of the present disclosure in the training set.

FIG. 8A is a graph showing the probability of any-cause rejection based on the 5-gene signature (F3, CD74, CXCL10, UBE2D2 and IFNA4) of the present disclosure in the training set.

FIG. 8B is a graph showing the probability of any-cause rejection based on the 5-gene signature (F3, CD74, CXCL10, UBE2D2 and IFNA4) of the present disclosure in the validation set.

FIG. 9 is a graph showing area under the curve-receiver operating characteristics analysis for the S-gene signature (HPRT1, CXCR4, CXCL10, IL32 and IFNA4) of the present disclosure in the training set.

FIG. 10 is a graph showing the probability of any-cause rejection based on the 5-gene signature (HPRT1, CXCR4, CXCL10, IL32 and IFNA4) of the present disclosure in the training set.

FIG. 11 is a graph showing urine exosome RNA stability.

FIG. 12 is a flow chart showing the results for the 192 biopsies that had matched urine samples used in Example 2 of the present disclosure.

FIG. 13 is a graph showing Receiver-Operating-Characteristic (ROC) curve for diagnosis of any-cause acute rejection. The ROC analysis and area under the curve (AUC) is shown for the exosome RNA signature described in Example 2 of the present disclosure and compared to the current standard of care parameters eGFR, and serum creatinine. The fraction of true positive results (sensitivity) and the fraction of false positive results (1 specificity) for diagnosis of any-cause acute rejection is displayed on the y-and x-axis, respectively. The AUC for the RNA signature is 0.90 (95% CI 0.84-0.96) and the AUC for eGFR is 0.59 (95%CI 0.50.67).

FIG. 14 is a waterfall plot of the urine exosome gene scores for identifying any-cause kidney transplant rejection as described in Example 2 of the present disclosure. The dotted line represents the cutoff value for the gene signature for any cause rejection, The arrows denote samples from clinically confirmed kidney rejection patients.

FIG. 15 is a graph showing Receiver-Operating-Characteristic (ROC) curve showing the fraction of true positive results (sensitivity) and the fraction of false positive results (1—specificity) for discriminating TCMR (T cell-mediated rejection, also referred to as cell-mediated kidney transplant rejection) from ABMR (antibody-mediated rejection, also referred to as antibody-mediated kidney transplant rejection), AUC 0.87 (95% CI 0.76-0.97) for the gene signature discussed in Example 2 of the present disclosure.

FIG. 16 is a waterfall plot of the urine exosome gene scores for identifying TCMR and ABMR as discussed in Example 2 of the present disclosure. The dotted line represents the cutoff value for the gene signature for discriminating between TCMR and ABMR. The arrows denote samples from clinically confirmed ABMR patients.

FIG. 17 is a graph showing the relative importance of each gene in the signature described in Example 2 of the present disclosure.

FIG. 18 is a graph showing the relative importance of each gene in the signature described in Example 2 of the present disclosure.

FIG. 19 is a graph showing Receiver-Operating-Characteristic (ROC) curve showing the fraction of true positive results (sensitivity) and the fraction of false positive results (1—specificity) for identifying cell-mediated kidney transplant rejection using the gene signature described in Example 3 of the present disclosure.

FIG. 20 is a waterfall plot of the urine exosome gene scores for identifying cell-mediated kidney transplant rejection as described in Example 3 of the present disclosure. The dotted line represents the cutoff value for the gene signature for identifying cell-mediated kidney transplant rejection. The arrows denote samples from clinically confirmed cell-mediated kidney transplant rejection patients.

FIG. 21 is a graph showing the relative importance of each gene in the signature described in Example 3 of the present disclosure.

FIG. 22 is a graph showing Receiver-Operating-Characteristic (ROC) curve showing the fraction of true positive results (sensitivity) and the fraction of false positive results (1—specificity) for identifying antibody-mediated kidney transplant rejection using the gene signature described in Example 4 of the present disclosure.

FIG. 23 is a waterfall plot of the urine exosome gene scores for identifying antibody-mediated kidney transplant rejection as described in Example 3 of the present disclosure. The dotted line represents the cutoff value for the gene signature for identifying antibody-mediated kidney transplant rejection. The arrows denote samples from clinically confirmed antibody-mediated kidney transplant rejection patients.

FIG. 24 is a graph showing the relative importance of each gene in the signature described in Example 4 of the present disclosure.

### DETAILED DESCRIPTION

Chronic kidney disease (CKD) is a major health concern in the Unites States and worldwide. While patients with end stage kidney disease (ESKD) require either dialysis or transplantation to sustain their life, the latter remains the treatment of choice. However, long term graft survival remains a major challenge due mostly to acute and chronic rejection. Although the rate of acute rejection has decreased in the modem era of potent immunosuppression, recent reported incidence of acute rejections in the literature ranges from 11 to 26%, During the first year after transplantation, the incidence of acute rejection is around 7.9%. This has been associated with a poor long-term allograft survival. The implementation of the Banff classification in 1991 provided a valuable tool for histopathological diagnosis of kidney transplant injury and allowed for standardization when comparing biopsy results between different studies. Serum creatinine (SCr), estimated glomerular filtration rate (eGFR) and urinary protein excretion are traditional biomarkers currently used to monitor the kidney allograft but they lack sensitivity, specificity and predictive ability. Kidney allograft biopsies with histopathological evaluation remain the gold standard to diagnose acute rejection. However, there are limitations to their use as they are invasive, costly and can be associated with significant morbidity. Several biomarkers have been identified as potential non-invasive tools to early diagnose graft rejection such as cell mRNA isolated from urine pellet. Recently, donor-derived cell-free DNA (dd-cfDNA) has been introduced to the clinical practice as a novel biomarker for graft rejection after solid organ transplantation. Despite results showing good performances in discriminating active rejection from no-rejection status, biopsies with T-cell mediated rejection (TCMR) subclass IA didn't reach the 1% dd-cfDNA cut-off required for diagnosis.

The present disclosure provides methods of identifying and treating kidney rejection in a subject comprising analyzing microvesicular RNA, cell-free DNA or the combination of microvesicular and cell-free DNA. Advantageously, the methods of the present disclosure can allow for the selection of treatment and/or treatment of an individual identified as having a kidney transplant rejection without the need for a renal biopsy, which can be an expensive, painful and potentially dangerous procedure.

Microvesicles are shed by eukaryotic and prokaryotic cells, or budded off from the plasma membrane, to the exterior of the cell. These membrane vesicles are heterogeneous in size with diameters ranging from about 10 nm to about 5000 nm. All membrane vesicles shed by cells <0.8 μm in diameter are referred to herein collectively as “exosomes,” “extracellular vesicles,” or “microvesicles.” These extracellular vesicles (EVs) include microvesicles, microvesicle-like particles, prostasomes, dexosomes, texosotnes, ectosomes, oncosomes, apoptotic bodies, retrovirus-like particles, and human endogenous retrovirus (HERV) particles. Small microvesicles (approximately 10 to 1000 nm, and more often 30 to 200 nm in diameter) that are released by exocytosis of intracellular multivesicular bodies are referred to in the art as “microvesicles.” Microvesicles shed by cells are also herein referred to as “exosomes.”

Exosomes are known to contain nucleic acids, including various DNA and RNA types such as mRNA (messenger RNA), miRNA (micro RNA), tRNA (transfer RNA), piRNA (piwi-interacting RNA), snRNA (small nuclear RNA), snoRNA (small nucleolar RNA), and. rRNA (ribosomal RNA), various classes of long non-coding RNA, including long intergenic non-coding RNA (lincRNA) as well as proteins. Recent studies reveal that nucleic acids within microvesicles have a role as biomarkers. For example, WO 2009/100029 describes, among other things, the use of nucleic acids extracted from microvesicles in Glioblastoma. multiforme (GBM, a particularly aggressive form of cancer) patient serum for medical diagnosis, prognosis and therapy evaluation. WO 2009/100029 also describes the use of nucleic acids extracted from microvesicles in human urine for the same purposes. The use of nucleic acids extracted from microvesicles is considered to potentially circumvent the need for biopsies, highlighting the enormous diagnostic potential of microvesicle biology (Skog et al. Nature Cell Biology, 2008. 10(12): 1470-1476.

Microvesicles can be isolated from liquid biopsy samples from a subject, involving biofluids such as whole blood, serum, plasma, urine, and cerebrospinal fluid (CSF). The nucleic acids contained within the microvesicles can subsequently be extracted. The extracted nucleic acids, e.g., microvesicular RNA (also referred to as exosomal RNA), can be further analyzed based on detection of a biomarker or a combination of biomarkers. The analysis can be used to generate a clinical assessment that diagnoses a subject with a disease, predicts the disease outcome of the subject, stratifies the subject within a larger population of subjects, predicts whether the subject will respond to a particular therapy, or determines if a subject is responding to an administered therapy.

The present disclosure provides a method of identifying kidney transplant rejection in a subject who has undergone a kidney transplant, the method comprising: a) determining the expression level of at least two of 15 biomarkers in microvesicular RNA isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least two of 15 biomarkers in microvesicular RNA isolated from a biological sample from the subject, wherein the 15 biomarkers comprise CXCL11 CD74, IL32, STAT1, CXCL14, SERPINA1, B2M, C3, PYCARD, BMP7, TBP, NAMPT, IFNGR1, IRAK2 and IL18BP; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of at least two of 15 biomarkers in microvesicular RNA and cell-free DNA (cfDNA) isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least two of 15 biomarkers in microvesicular RNA and cell-free DNA isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP; h) inputting the expression levels from step (a) into an algorithm to generate a score; c) determining the risk of a kidney transplant rejection in the subject based on the score.

In some aspects of the preceding methods, the kidney transplant rejection can be any-cause kidney transplant rejection.

In some aspects of the preceding methods, step (a) can comprise determining the expression level of at least three, or at least four, or at least five, or at least six, or at least seven, or at least eight, or at least nine, or at least 10, or at least 11, or at least 12, or at least 14 of the 15 biomarkers. In some aspects of the preceding methods, step (a) can comprise determining the expression level of each of the 15 biomarkers.

The present disclosure provides a method of identifying kidney transplant rejection in a subject who has undergone a kidney transplant, the method comprising: a) determining the expression level of:

(i) CXCL11, CD74, IL32, STAT 1, CXCL14, SERPINA1, B2M, C3 and PYCARD;

(ii) CXCL11, CD74, IL32, STAT1, CXCL14, SERP1NA1, B2M and C3;

(iii) CXCL11, CD74, IL32, STAT1, CXCL14, SERPINA1 and B2M;

(iv) CXCL11, CD74, IL32, STAT1, CXCL14 and SERPINA1;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determine the expression level of:

(i) CXCL11, CD74, IL32, STAT1, CXCL14, SERPINA1, B2M, C3 and PYCARD;

(ii) CDCL11, CD74, IL32, STAT1, CXCL14, SERPINA1, B2M and C3;

(iii) CXCL11. CD74, IL32, STAT1, CXCL14, SERPINA1 and B2M;

(iv) CXCL11, CD74, IL32, STAT1, CXCL14 and SERPINA1;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, CXCL14, SERPINA1, B2M, C3 and PYCARD;

(ii) CXCL11, CD74, IL32, STAT1, CXCL14, SERPINA1, B2M and C3;

(iii) CXCL11, CD74, IL32, STAT1, CXCL14, SERPINA1 and B2M;

(iv) CXCL11, CD74, IL32, STAT1, CXCL14 and SERPINA1;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a. kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, CXCL14, SERPINA1, B2M, C3 and PYCARD;

(ii) CXCL11, CD74, IL32, STAT1, CXCL14, SERPINA1 B2M and. C3;

(iii) CXCL11, CD74, IL32, STAT1, CXCL14, SERPINA1 and B2M;

(iv) CXCL11, CD74, IL32, STAT1, CXCL14 and SERPINA1;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of:

(i) CXCL11, CD74, IL32, STAT1, CXCL14, SERPINA1, B2M, C3 and PYCARD;

(ii) CXCL11, CD74, IL32, STAT1, CXCL14, SERPINA1, B2M and C3;

(iii) CXCL11, CD74, IL32, STAY1, CXCL14, SERPINA1 and B2M;

(iv) CXCL11, CD74, IL32, STAT1, CXCL14 and SERPINA1;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA and cell-free DNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a. kidney transplant rejection in a subject who has undergone a kidney transplant, the method comprising: a) determining the expression level of:

(i) CXCL11, CD74, IL32, STAT1, CXCL14, SERPINA1, B2M, C3 and PYCARD;

(ii) CXCL11, CD74, IL32, STAT1, CXCL14, SERPINA1, B2M and C3;

(iii) CXCL11, CD74, IL32, STAT1, CXCL14, SERPINA1 and B2M;

(iv) CXCL11, CD74, IL32, STAT1, CXCL14 and SERPINA1;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA and cell-free DNA isolated from a biological sample from the subject; b) inputting the expression levels from step (a) into an algorithm to generate a score; c) determining the risk of a kidney transplant rejection in the subject based on the score,

The present disclosure provides a method of identifying kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, CXCL14, SERVINAL B2M, C3 and PYCARD;

(ii) CXCL11, CD74, IL32, STAT1, CXCL14, SERPINA1, B2M. and C3;

(iii) CXCL11, CD74, IL32, STAT1, CXCL14, SERPINA1 and B2M;

(iv) CXCL11, CD74, IL32, STAT1, CXCL14 and SERPINAI;

(v) CXCL11, CD74, IL32, STAT1 and CXCL14;

(vi) CXCL11, CD74, IL32 and STAT1;

(vii) CXCL11, CD74, and IL32; or

(viii) CXCL11 and CD74

in microvesicular RNA and cell-free DNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, CXCL14, SERPINA1, B2M, C3 and PYCARD;

(ii) CXCL11, CD74, IL32, STAT1, CXCL14, SERPINA1, B2M and C3;

(iii) CXCL11, CD74, IL32, STAT1, CXCL14, SERPINA1, and B2M;

(iv) CXCL11, CD74, IL32, STAT1, CXCL14 and SERPINA1;

(v) CXCL11, CD74, IL32, STAT1, and CXCL14;

(vi) CXCL11, CD74, IL32 and STAT1;

(vii) CXCL11, CD74, and IL32; or

(viii) CXCLI I and CD74

in microvesicular RNA and cell-free DNA isolated from a biological sample from the subject; b) inputting the expression levels from step (a) into an algorithm to generate a score; c) determining the risk of a kidney transplant rejection in the subject based on the score.

In some aspects of the preceding methods, step (a) can comprise determining the expression level of CXCL11, CD74, IL32, STAT1 CXCL14, SERPINA1, B2M, C3 and PYCARD.

In some aspects of the preceding methods, step (a) can comprise determining the expression level of CXCL11, CD74, IL32, STAT1, CXCL14, SERPINA1, B2M and C3.

In some aspects of the preceding methods, step (a) can comprise determining the expression level of CXCL11, CD74, IL32, STAT1, CXCL14, SERPINA1 and B2M.

In some aspects of the preceding methods, step (a) can comprise determining the expression level of CXCL11, CD74, IL32, STAT1, CXCL14 and SERPINA1.

In some aspects of the preceding methods, step (a) can comprise determining the expression level of CXCL 11, CD74, IL32, STAT1 and CXCL14.

In some aspects of the preceding methods, step (a) can comprise determining the expression level of CXCL11, CD74, IL32 and STAT1.

In some aspects of the preceding methods, step (a) can comprise determining the expression level of CXCL11, CD74 and IL32.

In some aspects of the preceding methods, step (a) can comprise determining the expression level of CXCL11 and CD74.

The present disclosure provides a method of identifying kidney transplant rejection in a subject who has undergone a kidney transplant, the method comprising: a) determining the expression level of:

(i) CXCL11, STAT1, CXCL14, C3, PYCARD, BMP7, IFNGR1, IRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2;

(iv) CXCL11, CD74, IL32, STAT1, CXCL14, C3, PYCARD, BMP7, IFNGR1, IRAK2;

(v) CXCL11, CD74, STAT1, CXCL14, C3, PYCARD, BMP7, IFNGR1, IRAK2;

(vi) CD74, IL32, STAT1, CXCL14, C3, PYCARD, BMP7, IFNGR1, IRAK2; or

(vii) CXCL11, IL32, STAT1 CXCL14, C3, PYCARD, BMP7. IFNGR1, IRAK2 in microvesicular RNA isolated from a biological sample from the subject; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of:

(i) CXCL11, STAT1, CXCL14, C3, PYCARD, BMP7, IFNGR1, TRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2;

(iv) CXCL11, CD74, IL32, STAT1, CXCL14, C3, PYCARD, BMP7, IFNGR1, IRAK2;

(v) CXCL 11, CD74, STAT1, CXCL 14, C3, PYCARD, BMP7, IFNGR1, IRAK2;

(vi) CD74, IL32, STAT1, CXCL14, C3, PYCARD, BMP7, IFNGR1, IRAK2, or

(vii) CXCL11, IL32, STAT1, CXCL14, C3, PYCARD, BMP7, IFNGR1, IRAK2

in microvesicular RNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CXCL11, STAT1, CXCL14, C3, PYCARD, BMP7, IRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2,

(iv) CXCL11, CD74, IL32, STAT1, CXCL14, C3, PYCARD, BMP7, IFNGR1, IRAK2;

(v) CXCL11, CD74, STAT1. CXCL14, C3, PYCARD, BMP7, IFNGR1, IRAK2;

(vi) CD74, IL 32, STAT1, CXCL14, C3, PYCARD, BMP7, IFNGR1, IRAK2; or

(vii) CXCL11, IL32, STAT1 CXCL14, C3, PYCARD, BMP7, IFNGR1, IRAK2 in microvesicular RNA isolated from a biological sample from the subject; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CXCL11, STAT1, CXCL14, C3, PYCARD, BMP7, IFNGR1, IRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2;

(iv) CXCL11, CD74, IL32, STAT1, CXCL 14, C3, PYCARD, BMP7, IFNGR1, IRAK2;

(v) CXCL11, CD74, STAT1, CXCL14, C3, PYCARD, BMP7, IFNGR1, IRAK2;

(vi) CD74, IL32, STAT1, CXCL14, C3, PYCARD, BMP7, IFNGR1, IRAK2; or

(vii) CXCL11, IL32, STAT1, CXCL14, C3, PYCARD, BMP7, IFNGR1, IRAK2 in microvesicular RNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of:

(i) CXCL11, STAT1, CXCL14, C3, PYCARD, BMP7, IRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2;

(iv) CXCL11, CD74, IL32, STAT1, CXCL14, C3, PYCARD, BMP7, IFNGR1, IRAK2;

(v) CXCL11, CD74, STAT1, CXCL14, C3, PYCARD, BMP7, IRAK2;

(vi) CD74, IL32, STAT1, CXCL14, C3, PYCARD, BMP7, IFNGR1, IRAK2; or

(vii) IL32, STAT1, CXCL14, C3, PYCARD, BMP7, IFNGR1, IRAK2 in microvesicular RNA and cell-free DNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of:

(i) CXCL11, STAT1, CXCL14, C3, PYCARD, BMP7, IFNGR1, IRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2;

(iv) CXCL11, CD74, IL32, STAT1, CXCL14, C3, PYCARD, BMP7, IFNGR1, IRAK2;

(v) CXCL 11, CD74, STAT1, CXCL 14, C3, PYCARD, BMP7, IRAK2;

(vi) CD74, IL32, STAT1, CXCL14, C3, PYCARD, BMP7, IFNGR1, IRAK2; or

(vii) CXCL11, IL32, STAT1, CXCL14, C3, PYCARD, BMP7, IFNGR1, IRAK2 in microvesicular RNA and cell-free DNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CXCL11, STAT1, CXCL14, C3, PYCARD, BMP7, IFNGR1, IRAK2;

(ii) CD74, STAT1, CXCL14, C3, PYCARD, BMP7, IFNGR1, IRAK2;

(iii) 1132, STAT1, CXCL 4, C3, PYCARD, BMP7, IFNGR1, IRAK2;

(iv) CXCL11, CD74, IL32, STAT1, CXCL14, C3, PYCARD, BMP7, IFNGR1, IRAK2;

(v) CXCL11, CD74, STAT1, CXCL14, C3, PYCARD, BMP7, IFNGR1, IRAK2;

(vi) CD74, IL32, STAT1, CXCL14, C3, PYCARD, BMP7, 1FNGR.1, IRAK2; or

(vii) CXCL11, IL32, STAT1, CXCL14, C3, PYCARD, BMP7, IFNGR1, IRAK2 in microvesicular RNA and cell-free DNA isolated from a biological sample from the subject; h) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CXCL11, STAT1, CXCL14, C3, PYCARD, BMP7, IFNGR1, TRAK2;

(ii) CD74, STAT1, CXCL14, C3, PYCARD, BMP7, IFNGR1, IRAK2;

(iii) IL32, STAT1, CXCL14, C3, PYCARD, BMP7, IFNGR1, IRAK2;

(iv) CXCL11, CD74, IL32, STAT1, CXCL14, C3, PYCARD, BMP7, IFNGR1, IRAK2;

(v) CXCL11, CD74, STAT1, CXCL14, C3, PYCARD, BMP7, IFNGR1, IRAK2;

(vi) CD74, IL32, STAT1, CXCL14, C3, PYCARD, BMP7, IFNGR1, IRAK12, or

(vii) CXCL11, IL32, STAT1, CXCL14, C3, PYCARD, BMP7, IFNGR1, IRAK2 microvesicular RNA and cell-free DNA isolated from a biological sample from the subject; b) inputting the expression levels from step (a) into an algorithm to generate a score; c) determining the risk of a kidney transplant rejection in the subject based on the score.

In some aspects of the preceding methods, step (a) can comprise determining the expression level of CXCL11, STAT1, CXCL14, C3, PYCARD, BMP7, IFNGR1, IRAK2.

In some aspects of the preceding methods, step (a) can comprise determining the expression level of CD74, STAT1, CXCL14, C3, PYCARD, BMP7, IFNGR1, IRAK2.

In some aspects of the preceding methods, step (a) can comprise determining the expression level of IL32, STAT1, CXCL14, C3, PYCARD, BMP7, IFNGR1, IRAK2.

In some aspects of the preceding methods, step (a) can comprise determining the expression level of CXCL11, CD74, IL32, STAT1, CXCL14, C3, PYCARD, BMP7, IFNGR1, IRAK2.

In some aspects of the preceding methods, step (a) can comprise determining the expression level of CXCL11, CD74, STAT1, CXCL14, C3, PYCARD, BMP7, IFNGR1,

In some aspects of the preceding methods, step (a) can comprise determining the expression level of CD74, IL32, STAT1, CXCL14, C3, PYCARD, BMP7, IFNGR1, IRAK2,

In some aspects of the preceding methods, step (a) can comprise determining the expression level of CXCL11, IL32, STAT1, CXCL14, C3, PYCARD, BMP7, IFNGR1, IRAK2.

The present disclosure provides a method of identifying kidney transplant rejection in a subject who has undergone a kidney transplant, the method comprising: a) determining the expression level of:

(i) CXCL11, CD74, IL32, STAT1, SERPINA1, B2M, TBP, NAMPT, IL18BP;

(ii) CXCL11, CD74, IL32, CXCL14, SERPINA1, B2M, TBP, NAMPT, IL18BP;

(iii) CXCL11, CD74, IL32, SERPINA1, B2M, C3, TBP, NAMPT, IL18BP;

(iv) CXCL11, CD74, IL32, SERPINA1, B2M, PYCARD, TBP, NAMPT, IL18BP;

(v) CXCL11, CD74, IL32, SERPINA1, B2M, BMP7, TBP, NAMPT, IL18BP;

(vi) CXCL11, CD74, IL32, SERPINA1, B2M, TBP, NAMPT, IFNGR1, IL18BP; or

(vii) CXCL11, CD74, IL32, SERPINA1, B2M, TBP, NAMPT, IRAK2, IL 18BP in microvesicular RNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of:

(i) CXCL11, CD74, IL32, STAT1, SERPINA1, TBP, NAMPT, IL18BP;

(ii) CXCL11, CD74, IL32, CXCL14, SERPINA1, B2M, TBP, NAMPT, IL18BP;

(iii) CXCL11, CD74, IL32, SERPINA1, B2M, C3, TBP, NAMPT, IL18BP;

(iv) CXCL11, CD74, IL32, SERPINA1, B2M, PYCARD, TBP, NAMPT, IL18BP;

(v) CXCL11, CD74, IL32, SERPINA1, B2M, BMP7, TBP, NAMPT, IL18BP;

(vi) CXCL11, CD74, IL32, SERPINA1, B2M, TBP, NAMPT, IFNGR1, IL18BP; or

(vii) CXCL11, CD74, IL32, SERPINA1, B2M, TBP, NAMPT, IRAK2, IL18BP in microvesicular RNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of a.t least one, or at least two, or at least three, or at least four, or at least live, or at least six, or at least seven, or at least eight, or at least nine genes in at least one of the following gene sets:

(i) CXCL11, CD74, IL32, STAT1, SERPINAL B2M, TBP, NAMPT, IL18BP;

(ii) CXCL11, CD74, IL32, CXCL14, SERPINA1, B2M, TBP, NAMPT, IL18BP;

(iii) CXCL11, CD74, IL32, SERPINA1, B2M, C3, TBP, NAMPT, IL18BP;

(iv) CXCL11, CD74, IL32, SERPINA1, B2M, PYCARD, TBP, NAMPT, IL18BP;

(v) CXCL11, CD74, IL32, SERPINA1, B2M, BMP7, TBP, NAMPT, IL18BP;

(vi) CXCL11, CD74, IL32, SERPINA1, B2M, TBP, NAMPT, IFNGR1, IL 18BP; or

(vii) CXCL11, CD74, IL32, SERPINA1, B2M, TBP, NAMPT, IRAK2, IL18BP in microvesicular RNA isolated from a biological sample from the subject; b) inputting the expression levels from step (a) into an algorithm to generate a score; c) comparing the score to a predetermined cutoff value; and d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, SERPINA1, B2M, TBP, NAMPT, IL18BP;

(ii) CXCL11, CD74, IL32, CXCL14, SERPINA1, B2M, TBP, NAMPT,

(iii) CXCL11, CD74, IL32, SERPINA1, B2M, C3, TBP, NAMPT, II,18BP;

(iv) CXCL11, CD74, IL32, SERPINA1, B2M, PYCARD, TBP, NAMPT, IL18BP;

(v) CXCL11, CD74, IL32, SERPINA1, B2M, BMP7, TBP, NAMPT, IL18BP;

(vi) CXCL11, CD74, IL32, SERPINA1, B2M, TBP, NAMPT, IL18BP; or

(vii) CDCL11, CD74,1L32, SERPINA1, B2M, TBP, NAMPT, IRAK2, IL18BP microvesicular RNA isolated from a biological sample from the subject; b) inputting the expression levels from step (a) into an algorithm to generate a score; c) determining the risk of a kidney transplant rejection in the subject based on the score.

The present disclosure provides a method of identifying kidney transplant rejection a subject who has undergone a kidney transplant, the method comprising: a) determining the expression level of:

(i) CXCL11, CD74, IL32, STATT, SERPINA1, B2M, TBP, NAMPT, IL18BP;

(ii) CXCL11, CD74, IL32, CXCL14, SERPINA1, B2M, TBP, NAMPT, IL18BP;

(iii) CXCL11, CD74, IL32, SERPINA1, B2M, C3, TBP, NAMPT, IL18BP;

(iv) CXCL11, CD74, IL32., SERPINA1, B2M, PYCARD, TBP, NAMPT, IL18BP;

(v) CXCL11 CD74, IL32, SERPINA1, B2M, BMP7, TBP, NAMPT, IL18BP;

(vi) CXCL11, CD74, IL32, SERPINA1, B2M, TBP, NAMPT, IFNGR1, IL18BP; or

(vii) CXCL11, CD74, IL32, SERPINA1, B2M, TBP, NAMPT, IRAK2, IL18BP in microvesicular RNA and cell-free DNA isolated from a biological sample from the subject; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of:

(i) CXCL11, CD74, IL32, STAT 1, SERPINA1, TBP, NAMPT, IL18BP;

(ii) CXCL11, CD74, IL32, CXCL14, SERPINAI. B2M, TBP, NAMPT, IL18BP;

(iii) CXCL11, CD74, IL32, SERPINA1, B2M, C3. TBP, NAMPT, II,18BP;

(iv) CXCL11, CD74, IL32. SERPINA1, B2M, PYCARD, TBP, NAMPT, IL18BP,

(v) CXCL11, CD74, IL32, SERPINA1, B2M, BMP7, TBP, NAMPT, IL18BP;

(vi) CXCL11, CD74, IL32. SERPINA1, B2M, TBP, NAMPT, IFNGR1, IL 8BP; or

(vii) CXCL11, CD74, IL32, SERPINA1, B2M, TBP, NAMPT, IRAK2, IL18BP in microvesicular RNA and cell-free DNA isolated from a biological sample from the subject; h) inputting the expression levels from step (a) into an algorithm to generate a score; c) determining the risk of a kidney transplant rejection in the subject based on the score.

The present disclosure provides a method of identifying kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, SERPINA1, B2 M, TBP, NAMPT, IL18BP;

(ii) CXCL11, CD74, IL32, CXCL14, SERPINA1, B2M, TBP, NAMPT, 1L18BP;

(iii) CXCL11, CD74, IL32, SERPINA1, B2M, C3, TBP, NAMPT, IL18BP;

(iv) CXCL11, CD74, IL32, SERPINA1, B2M, PYCARD, TBP, NAMPT, IL18BP;

(v) CXCL11, CD74, IL32, SERPINA1, B2M, BMP7, TBP, NAMPT, IL18BP,

(vi) CXCL11, CD74,1L32, SERPINA1, B2M, TBP, NAMPT, IFNGR1, IL18BP; or

(vii) CXCL11, CD74, IL32, SERPINA1, B2M, TBP, NAMPT, IRAK2, IL1.8BP in microvesicular RNA and cell-free DNA isolated from a biological sample from the subject; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of determining the risk of a kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CXCL11, CD74, IL32, STAT1, SERPINA1, B2M, TBP, NAMPT, IL18BP;

(ii) CXCL11, CD74, IL32, CXCL14, SERPINA1, B2M, TBP, NAMPT, IL18BP;

(iii) CXCL11, CD74, IL32, SERPINA1, B2M, C3, TBP, NAMPT, IL18BP,

(iv) CXCL11, CD74, IL32, SERPINA1, B2M, PYCARD, TBP, NAMPT, IL18BP;

(v) CXCL11, CD74, IL32, SERPINA1, B2M, BMP7, TBP, NAMPT, IL18BP;

(vi) CXCL11, CD74, IL32, SERPINA1, B2M, TBP, NAMPT, IFNGR1, IL18BP; or

(vii) CXCL11, CD74, IL32, SERPINA1, B2M, TBP, NAMPT, IRAK2, IL18BP in microvesicular RNA and cell-free DNA isolated from a biological sample from the subject; h) inputting the expression levels from step (a) into an algorithm to generate a score; c) determining the risk of a kidney transplant rejection in the subject based on the score.

In some aspects of the preceding methods, step (a) can comprise determining the expression level of CXCL11, CD74, IL32, STAT1, SERPINA1, B2M, TBP, NAMPT, IL18BP.

In some aspects of the preceding methods, step (a) can comprise determining the expression level of CXCL11, CD74, IL32, CXCL14, SERPINA1, B2M, TBP, NAMPT, IL18BP.

In some aspects of the preceding methods, step (a) can comprise determining the expression level of CDCL11, CD74, IL32, SERPINA1, B2M, C3, TBP, NAMPT, IL18BP.

In some aspects of the preceding methods, step (a) can comprise determining the expression level of CXCL11, CD74, IL32, SERPINA1, B2M, PYCARD, TBP, NAMPT, IL18BP.

In some aspects of the preceding methods, step (a) can comprise determining the expression level of CXCL11, CD74, IL32, SERPINA1, B2M, BMP7, TBP, NAMPT, IL18BP

In some aspects of the preceding methods, step (a) can comprise determining the expression level of CXCI.11, CD74, IL32, SERPINA1 B2M, TBP, NAMPT, IFNGR1, IL18BP.

In some aspects of the preceding methods, step (a) can comprise determining the expression level of CXCL11, CD74, IL32, SERPINA1, B2M, TBP, NAMPT, IRAK2, IL18BP.

The present disclosure provides a method of identifying cell-mediated kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CD74, CXCL11, C3, CCL2, B2M, IL15, IL18BP, FPR2, ALOX5AP, IL1RAP, TLR1, NAMPT and IL1R2; b) inputting the expression levels from step (a) into an algorithm to generate a score; c) comparing the score to a predetermined cutoff value; d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CD74, CXCL11, C3, CCL2, B2M, IL15, IL18BP, FPR2, ALOX5AP, IL1RAP, TLR1, NAMPT and IL1R2; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of at least two of 13 biomarkers in microvesicular RNA and cell-free DNA isolated from a biological sample from the subject, Wherein the 13 biomarkers comprise CD74, CXCL11, C3, CCL2, B2M, IL15, IL18BP, FPR2, ALOX5 AP, IL1RAP, TLR1, NAMPT and IL1R2; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least two of 13 biomarkers in microvesicular RNA and cell-free DNA isolated from a biological sample from the subject, wherein the 13 biomarkers comprise CD74, CXCL11, C3, CCL2, B2M, IL15, IL18BP, FPR2, ALOX5AP, IL1RAP, TLR1, NAMPT and IL1R2; b) inputting the expression levels from step (a) into an algorithm to generate a score; c) determining the risk of a cell-mediated kidney transplant rejection in the subject based on the score.

In some aspects of the preceding methods, step (a) can comprise determining the expression level of at least three, or at least four, or at least five, or at least six, or at least seven, or at least eight, or at least nine, or at least 10, or at least II, or at least 12 of the 13 biomarkers.

In sonic aspects of the preceding methods, step (a) can comprise determining the expression level of each of the 13 biomarkers.

The present disclosure provides a method of identifying cell-mediated kidney transplant rejection in a subject who has undergone a kidney transplant, the method comprising: a) determining the expression level of:

(i) CD74, CXCL11, C3, CCL2, B2M, IL15, IL18BP and FPR2;

(ii) CD74, CXCL11, C3, CCL2, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11

in microvesicular RNA isolated from a biological sample from the subject; b) inputting the expression levels from step (a) into an algorithm to generate a score; c) comparing the score to a predetermined cutoff value; d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of:

(i) CD74, CXCI.11, C3, CCL2, B2M, IL15, IL18BP and FPR2;

(ii) CD74, CXCL11, C3, CCL2, B2M, IL15 and IL18BP;

(iii) CD74, CXCL11, C3, CCL2, B2M and IL15,

(iv) CD74, CXCL11, C3, CCL2 and B2M;

(v) CD74, CXCL11, C3 and CCL2;

(vi) CD74, CXCL11 and C3; or

(vii) CD74 and CXCL11;

microvesicular RNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD74, CXCL11, C3, CCL2, B2M, IL15, IL18BP and FPR2.;

(ii) CD74, CXCL11, C3, CCL2, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11

in microvesicular RNA isolated from a biological sample from the subject; b) inputting the expression levels from step (a) into an algorithm to generate a score; c) comparing the score to a predetermined cutoff value; d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD74, CXCL11, C3, CCL2, B2M, IL15, IL18BP and FPR2;

(ii) C1)74, CXCL11, C3, CCL2, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11

in microvesicular RNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of:

(i) CD74, CXCL11, C3, CCL2, B2M, IL15, IL18BP and FPR2;

(ii) CD74, CXCL11, C3, CCL2, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11

microvesicular RNA and cell-free DNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of:

(i) CD74, CXCL11, C3, CCL2, B2M, IL15, IL18BP and FPR2;

(ii) CD74, CXCL11, C3, CCL2, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11;

in microvesicular RNA and cell-free DNA isolated from a biological sample from the subject; 11) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD74, CXCL11, C3. CCL2, B2M, IL15, IL18BP and FPR2;

(ii) CD74, CXCL11, C3, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11

in microvesictilar RNA and cell-free DNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD74, CXCL11, C3, CCL2, B2M, IL15, IL18BP and FPR2;

(ii) CD74, CXCL11, C3, CCL2, B2M, IL15 and IL18BP;

(iii) CD74, CXCL11, C3, CCL2, B2M and IL15;

(iv) CD74, CXCL11, C3, CCL2 and B2M;

(v) CD74, CXCL11, C3 and CCL2;

(vi) CD74, CXCL11 and C3; or

(vii) CD74 and CXCL11;

in microvesicular RNA and cell-free DNA isolated from a biological sample from the subject; h) inputting the expression levels from step (a) into an algorithm to generate a score; c) determining the risk of a cell-mediated kidney transplant rejection in the subject based on the score.

In some aspects of the preceding methods, step (a) can comprise determining the expression level of CD74, CXCL11, C3, CCL2, B2M, IL15, IL18BP and FPR2.

In some aspects of the preceding methods, step (a) can comprise determining the expression level of CD74, CXCL11, C3, CCL2, B2M, IL15 and IL18BP,

In some aspects of the preceding methods, step (a) can comprise determining the expression level of CD74, CXCL11, C3, CCL2, B2M and IL15.

In some aspects of the preceding methods, step (a) can comprise determining the expression level of CD74, CXCL11, C3, CCL2 and B2M.

In some aspects of the preceding methods, step (a) can comprise determining the expression level of CD74, CXCL11, C3 and CCL2.

In some aspects of the preceding methods, step (a) can comprise determining the expression level of CD74, CXCL11 and C3.

In some aspects of the preceding methods, step (a) can comprise determining the expression level of CD74 and CXCL11.

The present disclosure provides a method of identifying cell-mediated kidney transplant rejection in a subject who has undergone a. kidney transplant, the method comprising: a) determining the expression level of:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP;

(v) CD74, CXCL11, C3, B2M, IL1RAP;

(vi) CD74, CXCL11, C3, IL15, IL1RAP;

(vii) CD74, CXCL11, C3, IL18BP, IL1RAP;

(viii) CD74, CXCL11, C3, FPR2, IL1RAP; or

(ix) CD74, CXCL11, C3, ALOX5AP, IL1RAP

microvesicular RNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP;

(v) CD74, CXCL11, C3, B2M, IL1RAP;

(vi) CD74, CXCL11, C3, It15, IL1RAP;

(vii) CD74, CXCL11, C3, IL18BP, IL1RAP;

(viii) CD74, CXCL11, C3, FPR2, IL1RAP; or

(ix) CD74, CXCL11, C3, ALOX5AP, IL1RAP;

in microvesicular RNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of at least one, or at least two, or at least three, or at least four, or at least five genes in at least one of the following gene sets:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP;

(v) CD74, CXCL11, C3, B2M, IL1RAP;

(vi) CD74, CXCL,11, C3, IL15, IL1RAP;

(vii) CD74, CXCL11, C3, IL18BP, IL1RAP;

(viii) CD74, CXCL11, C3, FPR2, IL1RAP; or

(ix) CD74, CXCL11, C3, ALOX5AP, IL1RAP

in microvesicular RNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least one, or at least two, or at least three, or at least four, or at least five genes in at least one of the following gene sets:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP;

(v) CD74, CXCL11, C3, B2M, IL1RAP;

(vi) CD74, CXCL11, C3, IL15, IL1RAP;

(vii) CD74, CXCL11, C3, IL18BP, IL1RAP;

(ix) CD74, CXCL11, C3, ALOX5AP, IL1RAP;

in microvesicular RNA isolated from a biological sample from the subject; h) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a. kidney transplant, the method comprising: a) determining the expression level of:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP,

(v) CD74, CXCL11, C3, B2M, IL1RAP;

(vi) CD74, CXCL11, C3, II:15, IL1RAP;

(vii) CD74, CXCL11, C3, IL18BP, IL1RAP;

(viii) CD74, CXCL11, C3, FPR2, IL1RAP; or

(ix) CD74, CXCL11, C3, ALOX5AP, IL1RAP

in microvesicular RNA and cell-free DNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP;

(v) CD74, CXCL11, C3, B2M, IL1RAP;

(vi) CD74, CXCL1 I, C3, IL15, IL1RAP;

(vii) CD74, CXCL11, C3, IL18BP, IL1RAP;

(viii) CD74, CXCL11, C3, FPR2, IL1RAP; or

(ix) CD74, CXCL11, C3, ALOX5AP, IL1RAP;

in microvesicular RNA and cell-free DNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a. subject who has undergone a. kidney transplant, the method comprising: a) determining the expression level of at least one, or at least two, or at least three, or at least four, or at least five genes in at least one of the following gene sets:

(i) CD74, CXCL11, C3, IL1RAP;

(ii) CD74 C3, IL1RAP;

(iii) CXCL11, C3, IL1RAP;

(iv) CD74, CXCL11, C3, CCL2, IRAP;

(v) CD74, CXCL11, C3, B2M, IL1RAP;

(vi) CD74, CXCL11, C3, IL15, IL1RAP,

(vii) CD74, CXCL11, C3, IL18BP, IL1RAP,

(viii) CD74, CXCL11, C3, FPR2, IL1RAP; or

(ix) CD74, CXCL11, C3-ALOX5AP, IL1RAP

in microvesicular RNA and cell-free DNA isolated from a biological sample from the subject; b) inputting the expression levels from step (a) into an algorithm to generate a score; c) comparing the score to a. predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least one, or at least two, or at least three, or at least four, or at least five genes in at least one of the following gene sets:

(i) CD74, CXCL11, C3, IL1RAP;

(ii) CD74, C3, IL1RAP;

(iii) CXCL11, C3, IL1RAP;

(iv) CD74, CXCL11, C3, CCL2, IL1RAP;

(v) CD74, CXCL11, C3, B2M, IL1RAP,

(vi) CD74, CXCL11, C3, IL15, IL1RAP;

(vii) CD74, CXCL11, C3, IL18BP, IL1RAP;

(viii) CD74, CXCL11, C3, FPR2, IL1RAP; or

(ix) CD74, CXCL11, C3, ALOX5AP, IL1RAP;

microvesicular RNA and cell-free DNA isolated from a biological sample from the subject; b) inputting the expression levels from step (a) into an algorithm to generate a score; c) determining the risk of a cell-mediated kidney transplant rejection in the subject based on the score.

In some aspects of the preceding methods, step (a) can comprise determining the expression level of CD74, CXCL11, C3, IL1RAP.

In some aspects of the preceding methods, step (a) can comprise determining the expression level of CD74, C3, IL1RAP,

In some aspects of the preceding methods, step (a) can comprise determining the expression level of CXCL11, C3, IL1RAP.

In some aspects of the preceding methods, step (a) can comprise determining the expression level of CD74, CXCL11, C3, CCL2, IL1RAP.

In some aspects of the preceding methods, step (a) can comprise determining the expression level of CD74, CXCL11, C3, B2M, IL1RAP.

In some aspects of the preceding methods, step (a) can comprise determining the expression level of CD74, CXCL11, C3, IL15, IL1RAP.

In some aspects of the preceding methods, step (a) can comprise determining the expression level of CD74, CXCL11, C3, IL18BP, IL1RAP.

In some aspects of the preceding methods, step (a) can comprise determining the expression level of CD74, CXCL11, C3, FPR2,

In some aspects of the preceding methods, step (a) can comprise determining the expression level of CD74, CXCL11, C3, ALOX5AP, IL1RAP.

The present disclosure provides a method of identifying cell-mediated kidney transplant rejection in a subject who has undergone a kidney transplant, the method comprising: a) determining the expression level of:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP, TLR ; NAMPT, IL1R2

in microvesicular RNA isolated from a biological sample from the subject; b) inputting the expression levels from step (a) into an algorithm to generate a score; c) comparing the score to a. predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP, TLR1, NAMPT, IL1R2

in microvesicular RNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten, or at least eleven, or at least twelve genes in at least one of the following gene sets:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP, TLR1, NAMPT, IL1R2

in microvesicular RNA isolated from a. biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a. subject who has undergone a kidney transplant, the method comprising: a) determining the expression level of at least one, or at least two, or at least three, or at least four, or at least five, or at least six, or at least seven, or at least eight, or at least nine, or at least ten, or at least eleven, or at least twelve genes in at least one of the following gene sets:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP TLR1, NAMPT, IL1R2

in microvesicular RNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL 15, IL18BP, FPR2, ALOX5AP, IL1RAP, TLR1, NAMPT, IL1R2

in microvesicular RNA and cell-free DNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP, TLR1, NAMPT, IL1R2

in microvesicular RNA and cell-free DNA isolated from a biological sample from the subject; b) inputting the expression levels from step (a) into an algorithm to generate a score; c) determining the risk of a cell-mediated kidney transplant rejection in the subject based on the score.

The present disclosure provides a method of identifying cell-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten, or at least eleven, or at least twelve genes in at least one of the following gene sets:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP, TLR1, NAMPT, IL1R2

in microvesicular RNA and cell-free DNA isolated from a biological sample from the subject; b) inputting the expression levels from step (a) into an algorithm to generate a score; c) comparing the score to a predetermined cutoff value; and d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

The present disclosure provides a method of determining the risk of a cell-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten, or at least eleven, or at least twelve genes in at least one of the following gene sets:

(i) CD74, CXCL11, C3, CCL2, B2M, IL15, IL18BP, FPR2, ALOX5AP, TLR1, NAMPT, IL1R2; or

(ii) CD74, CXCL11, CCL2, B2M, IL15, IL18BP, FPR2, ALOX5AP, IL1RAP, TLR1, NAMPT, IL1R2

in microvesicular RNA and cell-free DNA isolated from a biological sample from the subject; b) inputting the expression levels from step (a) into an algorithm to generate a score; c) determining the risk of a cell-mediated kidney transplant rejection in the subject based on the score.

In some aspects of the preceding methods, step (a) can comprise determining the expression level of CD74, CXCL11, C3, CCL2, B2M, IL15, IL18BP, FPR2, ALOX5AP, TLR1, NAMPT, IL1R2.

In some aspects of the preceding methods, step (a) can comprise determining the expression level of CD74. CXCL11, CCL2, B2M, IL15, IL18BP, FPR2, ALOX5AP, IL1RAP, TLR1, NAMPT, IL1R2.

The present disclosure provides a method of identifying antibody-mediated kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least two of 13 biomarkers in microvesicular RNA and cell-free DNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1; b) inputting the expression levels from step (a) into an algorithm to generate a score; c) comparing the score to a predetermined cutoff value; d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least two of 13 biomarkers in microvesicular RNA and cell-free DNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1; b) inputting the expression levels from step (a) into an algorithm to generate a score; c) determining the risk of an antibody-mediated kidney transplant rejection in the subject based on the score.

In some aspects of the preceding methods, step (a) can comprise determining the expression level of at least three, or at least four, or at least five, or at least six, or at least seven, or at least eight, or at least nine, or at least ten, or at least eleven, or at least twelve of the 13 biomarkers.

In some aspects of the preceding methods, step (a) can. comprise determining the expression level of each of the 13 biomarkers.

The present disclosure provides a method of identifying antibody-mediated kidney transplant rejection in a subject who has undergone a kidney transplant, the method comprising: a) determining the expression level of:

(i) CD44, NAMPT, PYCARD, IRAK2, IL32, TBP, BCL10, IFNGR1, BMP7 and STAT1;

(ii) CD44, NAMPT, PYCARD, IRAK2, IL32, TBP, BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, IL32 and TBP;

(vi) CD44, NAMPT, PYCARD, IRAK2 and IL32;

(vii) CD44, NAMPT, PYCARD and IRAK2,

(viii) CD44, NAMPT and PYCARD; or

(ix) CD44 and NAMPT

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of:

(i) CD44, NAMPT, PYCARD, IRAK2, IL32, TBP, BCL10, IFNGR1, BMP7 and STAT1;

(ii) CD44, NAMPT, PYCARD, IRAK2, IL32, TBP, BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, IL32 and TBP;

(vi) CD44, NAMPT, PYCARD, IRAK2 and IL32;

(vii) CD44, NAMPT, PYCARD and IRAK2,

(viii) CD44, NAMPT and PYCARD; or

(ix) CD44 and NAMPT

in microvesicular RNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CD44, NAMPT, PYCARD, IRAK2, IL32, TBP, BCL10, IFNGR1, BMP7 and STAT1;

(ii) CD44, NAMPT, PYCARD, IRAK2, 1L32, TBP, BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, IL32 and TBP;

(vi) CD44, NAMPT, PYCARD, IRAK2 and IL32;

(vii) CD44, NAMPT, PYCARD and IRAK2;

(viii) CD44, NAMPT and PYCARD; or

(ix) CD44 and NAMPT

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CD44, NAMPT, PYCARD, IRAK2, IL32, TBP, BCL10, IFNGR1, BMP7 and STAT1;

(ii) CD44, NAMPT, PYCARD, IRAK2, 1L32, TBP, BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, 1L32 and TBP;

(vi) CD44, NAMPT, PYCARD, IRAK2 and IL32;

(vii) CD44, NAMPT, PYCARD and IRAK2;

(viii) CD44, NAMPT and PYCARD; or

(ix) CD44 and NAMPT

in microvesicular RNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of

(i) CD44, NAMPT, PYCARD, IRAK2, IL32, TBP, BCL10, IFNGR1, BMP7 and STAT1;

(ii) CD44, NAMPT, PYCARD, IRAK2, IL32, TBP, BCL10, IFNGR1 and BMP7;

(iii) CD44, NAMPT, PYCARD, IRAK2, 1L32, TBP, BCL10 and IFNGR1;

(iv) CD44, NAMPT, PYCARD, IRAK2, 1L32, TBP and BCL10;

(v) CD44, NAMPT, PYCARD, IRAK2, IL32 and TBP;

(vi) CD44, NAMPT, PYCARD, IRAK2 and IL32;

(vii) CD44, NAMPT, PYCARD and IRAK2;

(viii) CD44, NAMPT and PYCARD; or

(ix) CD44 and NAMPT

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of:

(i) CD44, NAMPT, PYCARD, IRAK2, 1L32, TBP, BCL10, IFNGR1, BMP7 and STAT 1;

(ii) CD44, NAMPT, PYCARD, IRAK2, 1L32, TBP. BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, 1L32 and TBP;

(vi) CD44, NAMPT, PYCARD, IRAK2 and IL32;

(vii) CD44, NAMPT, PYCARD and IRAK2;

(viii) CD44, NAMPT and PYCARD; or

(ix) CD44 and NAMPT

in microvesicular RNA and cell-free DNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CD44, NAMPT, PYCARD, IRAK2, IL32, TBP, BCL10, IFNGR1, BMP7 and STAT1;

(ii) CD44, NAMPT, PYCARD, IRAK2, IL32, TBP, BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, IL32 and TBP;

(vi) CD44, NAMPT, PYCARD, IRAK2 and IL32;

(vii) CD44, NAMPT, PYCARD and IRAK2;

(viii) CD44, NAMPT and PYCARD; or

(ix) CD44 and NAMPT

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine, or at least ten genes in at least one of the following gene sets:

(i) CD44, NAMPT, PYCARD, IRAK2, IL32, TBP, BCL10, IFNGR1, BMP7 and STAT 1;

(ii) CD44, NAMPT, PYCARD, IRAK2, IL32, TBP, BCL10, IFNGR1 and BMP7;

(iii) CD44, NAMPT, PYCARD, IRAK2, IL32, TBP, BCL10 and IFNGR1;

(iv) CD44, NAMPT, PYCARD, IRAK2, IL32, TBP and BCL10;

(v) CD44, NAMPT, PYCARD, IRAK2, IL32 and TBP;

(vi) CD44, NAMPT, PYCARD, IRAK2 and IL32;

(vii) CD44, NAMPT, PYCARD and IRAK2;

(viii) CD44, NAMPT and PYCARD; or

(ix) CD44 and NAMPT

in microvesicular RNA and cell-free DNA isolated from a biological sample from the subject; b) inputting the expression levels from step (a) into an algorithm to generate a score; c) determining the risk of an antibody-mediated kidney transplant rejection in the subject based on the score.

In some aspects of the preceding methods, step (a) can comprise determining the expression level of CD44, NAMPT, PYCARD, IRAK2, IL32, TBP, BCL10, IFNGR1, BMP7 and STAT1.

In some aspects of the preceding methods, step (a) can comprise determining the expression level of CD44, NAMPT, PYCARD, IRAK2, IL32, TBP, BCL10, IFNGR1 and BMP7.

In some aspects of the preceding methods, step (a) can comprise determining the expression level of CD44, NAMPT, PYCARD, IRAK2, IL32, TBP, BCL10 and IFNGR1.

In some aspects of the preceding methods, step (a) can comprise determining the expression level of CD44, NAMPT, PYCARD, RAK2, IL32, TBP and BCL10.

In some aspects of the preceding methods, step (a) can comprise determining the expression level of CD44, NAMPT, PYCARD, IRAK2, IL32 and TBP.

In some aspects of the preceding methods, step (a) can comprise determining the expression level of CD44, NAMPT, PYCARD, IRAK2 and IL32.

In some aspects of the preceding methods, step (a) can comprise determining the expression level of CD44, NAMPT, PYCARD and IRAK2,

In some aspects of the preceding methods, step (a) can comprise determining the expression level of CD44, NAMPT and PYCARD.

In sonic aspects of the preceding methods, step (a) can comprise determining the expression level of CD44 and NAMPT.

The present disclosure provides a method of identifying antibody-mediated kidney transplant rejection in a subject who has undergone a kidney transplant, the method comprising: a) determining the expression level of:

(i) CD44, PYCARD, IRAK2, IL32, IFNGR1, BMP7, STAT1;

(ii) NAMPT, PYCARD, IL32, IFNGR1, BMP7, STAT1;

(iii) PYCARD, IRAK2, IL32, TBP, IFNGR1, BMP7, STAT1;

(iv) PYCARD, IRAK2, IL32, BCL10, IFNGR1, BMP7, STAT1;

(v) CD44, NAMPT, PYCARD, IRAK2, IL32, IFNGR1, BMP7, STAT1;

(vii) CD44, NAMPT, PYCARD, IRAK2. IL32, BCL10, IFNGR1, BMP7, STAT1; or

(vii) CD44, NAMPT, PYCARD, IRAK2, IL32, TBP IFNGR1, BMP7, STAT1

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a. method of determining the risk of an antibody-mediated kidney transplant rejection in a subject who has undergone a kidney transplant, the method comprising: a) determining the expression level of

(i) CD44, PYCARD, IRAK2, IL32, IFNGR1, BMP7, STAT1;

(ii) NAMPT, PYCARD, IRAK2, IL32, IFNGR1, BMP7, STAT1;

(iii) PYCARD, IRAK2, IL32, TBP, IFNGR1, BMP7, STAT1;

(iv) PYCARD, IRAK2, IL32, BCL10, IFNGR1, BMP7, STAT1;

(v) CD44, NAMPT, PYCARD, IRAK2, IL32, IFNGR1, BMP7, STAT1;

(vi) CD44, NAMPT, PYCARD, IRAK2, IL32, BCL10, IFNGR1, BMP7, STAT1; or

(vii) CD44, NAMPT, PYCARD, IRAK2, IL32, TBP, IFNGR1, BMP7, STAT1

in microvesicular RNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CD44, PYCARD, IRAK2, IL32, IFNGR1, BMP7, STAT1;

(ii) NAMPT, PYCARD, IRAK2, IL32, IFNGR1, BMP7, STAT1;

(iii) PYCARD, IRAK2, IL32, TBP, IFNGR1, BMP7, STAT1;

(iv) PYCARD, IRAK2, IL32, BCL10, IFNGR1, BMP7, STAT1;

(v) CD44, NAMPT, PYCARD, IRAK2, IL32, IFNGR1, BMP7, STAT1;

(vi) CD44, NAMPT, PYCARD, IRAK2, IL32, BCL10, IFNGR1, BMP7, STAT1; or

(vii) CD44, NAMPT, PYCARD, IRAK2, IL32, TBP, IFNGR1, BMP7, STAT1

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined. cutoff value.

The present disclosure provides a method of determining the risk of an antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CD44, PYCARD, IRAK2, IL32, IFNGR1, BMP7, STAT1;

(ii) NAMPT, PYCARD, IRAK2, IL32, IFNGR1, BMP7, STAT1;

(iii) PYCARD, IRAK2, IL32, TBP, IFNGR1, BMP7, STAT1;

(iv) PYCARD, IRAK2, IL32, BCL10, BMP7, STAT1;

(v) CD44, NAMPT, PYCARD, IL32, IFNGR1, BMP7, STAT1;

(vi) CD44, NAMPT, PYCARD, IRAK2, IL32, BCL10, IFNGR1, BMP7, STAT1; or

(vii) CD44, NAMPT, PYCARD, IRAK2, IL32, TBP, IFNGR1, BMP7, STAT1

in microvesicular RNA isolated from a biological sample from the subject; h) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of:

(i) CD44, PYCARD, IRAK2, IL32, IFNGR1, BMP7, STAT1;

(ii) NAMPT, PYCARD, TRAK2, IL32, IFNGR1, BMP7, STAT1;

(iii) PYCARD, IRAK2, IL32, TBP, IFNGR1, BMP7, STAT1;

(iv) PYCARD, IRAK2, IL32, BCL10, IFNGR1, BMP7, STAT1;

(v) CD44. NAMPT, PYCARD, IRAK2, IL32, IFNGR1, BMP7, STAT1;

(vi) CD44, NAMPT, PYCARD, IRAK2, IL32, BCL10, BMP7, STAT1; or

(vii) CD44, NAMPT, PYCARD, IRAK2, IL32, TBP, IFNGR1, BMP7, STAT1

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of:

(i) CD44, PYCARD, IRAK2, IL32, IFNGR1, BMP7, STAT1;

(ii) NAMPT, PYCARD, IRAK2, IL32, IFNGR1, BMP7, STAT1;

(iii) PYCARD, IRAK2, IL32, TBP, IFNGR1, BMP7, STAT1,

(iv) PYCARD, IRAK2, IL32, BCL10, IFNGR1, BMP7, STAT1;

(v) CD44, NAMPT, PYCARD, IRAK2, IL32, IFNGR1, BMP7, STAT1;

(vi) CD44, NAMPT, PYCARD, IRAK2, IL32, BCL10, IFNGR1, BMP7. STAT1; or

(vii) CD44, NAMPT, PYCARD, IRAK2, IL32, TBP, IFNGR1, BMP7, STAT1

in microvesicular RNA and cell-free DNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CD44, PYCARD, IRAK2, IL32, IFNGR1, BMP7, STAT1;

(ii) NAMPT, PYCARD, IRAK2, IL32, IFNGR1, BMP7, STAT1;

(iii) PYCARD, IRAK2, IL32, TBP, IFNGR1, IBMP7, STAT1;

(iv) PYCARD, IRAK2, IL32, BCL10, IFNGR1, BMP7, STAT1;

(v) CD44, NAMPT, PYCARD, IRAK2, IL32, IFNGR1, BMP7, STAT1;

(vi) CD44, NAMPT, PYCARD, IRAK2, 1L32, BCL10, IFNGR1, BMP7, STAT1: or

(vii) CD44, NAMPT, PYCARD, IRAK2, IL32, TBP IFNGR1, BMP7, STAT1

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least one, or at least two, or at least three, or at least four, or at least five, or at least six, or at least seven, or at least eight, or at least nine genes in at least one of the following gene sets:

(i) CD44, PYCARD, IRAK2, IL32, IFNGR1, BMP7, STAT1;

(ii) NAMPT, PYCARD, IRAK2, IL32, IFNGR1, BMP7, STAT1;

(iii) PYCARD, IRAK2, 1L32, TBP, IFNGR1, BMP7, STAT1;

(iv) PYCARD, IRAK2, 1L32, BCL10, IFNGR1, BMP7, STAT1;

(v) CD44, NAMPT, PYCARD, IRAK2, IL32, IFNGR1, BMP7, STAT1;

(vi) CD44, NAMPT, PYCARD, IRAK2, IL32, BCL10, IFNGR1, BMP7, STAT1; or

(vii) CD44, NAMPT, PYCARD, IRAK2, IL32, TBP, IFNGR1, BMP7, STAT1

in microvesicular RNA and cell-free DNA isolated from a biological sample from the subject; b) inputting the expression levels from step (a) into an algorithm to generate a score; c) determining the risk of an antibody-mediated kidney transplant rejection in the subject based on the score.

In some aspects of the preceding methods, step (a) can comprise determining the expression level of CD44, PYCARD, IRAK2, IL32, IFNGR1, BMP7, STAT1.

In some aspects of the preceding methods, step (a) can comprise determining the expression level of NAMPT, PYCARD, IRAK2, IL32, IFNGR1, BMP7, STAT1.

In some aspects of the preceding methods, step (a) can comprise determining the expression level of PYCARD, IRAK2, IL32, TBP, BMP7, STAT1.

In some aspects of the preceding methods, step (a) can comprise determining the expression level of PYCARD, h2, IL32, BCL10, BMP7, STAT1.

In sonic aspects of the preceding methods, step (a) can comprise determining the expression level of CD44, NAMPT, PYCARD, IRAK2, IL32, BMP7, STAT1.

In some aspects of the preceding methods, step (a) can comprise determining the expression level of CD44, NAMPT, PYCARD, IRAK2, IL32, BCL10, IFNGR1, BMP7, STAT 1.

In some aspects of the preceding methods, step (a) can comprise determining the expression level of CD44, NAMPT, PYCARD, IRAK2, IL32, TBP, IFNGR1, BMP7, STAT1.

The present disclosure provides a method of identifying antibody-mediated kidney transplant rejection in a subject who has undergone a kidney transplant, the method comprising: a) determining the expression level of:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44, NAMPT, IL32, TBP, BCL10, ANXA1, TYMP, NFX1;

(iv) CD44, NAMPT, TBP, BCL10, IFNGR1, ANXA1, TYMP, NFX1;

(v) CD44, NAMPT, TBP, BCL10, BMP7, ANXA1, TYMP, NFX1; or

(vi) CD44, NAMPT, TBP, BCL10, STAT1, ANXA1, TYMP, NFX1

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of

(i) CD44, NAMPT, PYCARD, TBP, BC10, ANXA1, TYMP, NFX1;

(ii) CD44, NAMPT, IRAK2, TBP, BCL10, ANXA1, TYMP, NFX1;

(iii) CD44, NAMPT, IL32, TBP, BCL10, ANXA1, TYMP, NFX1;

(iv) CD44, NAMPT, TBP, BCL10, IFNGR1, ANXA1, TYMP,

(v) CD44, NAMPT, TBP, BCL10, BMP7, ANXA1, TYMP, NFX1; or

(vi) CD44, NAMPT, TBP, BCL10, STAT1, ANXA1, TYMP, NFX1

in microvesicular RNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44, NAMPT, IL32, TBP, BCL10, ANXA1, TYMP, NFX1;

(iv) CD44, NAMPT, TBP, IFNGR1, ANXA1, TYMP, NFX1;

(v) CD44, NAMPT, TBP, BCL10, BMP7, ANXA1, TYMP, NFX1; or

(vi) CD44, NAMPT, TBP, BCL10, STAT1, ANXA1, TYMP, NFX1

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44, NAMPT, 1132, TBP, BCL10, ANXA1, TYMP, NFX1;

(iv) CD44, NAMPT, TBP, BCL10, IFNGR1, ANXA1, TYMP, NFX1;

(v) CD44, NAMPT, TBP, BCL10, BMP7, ANXA1, TYMP, NFX1; or

(vi) CD44, NAMPT, TBP, BCL10, STAT1, ANXA1, TYMP, NFX1

in microvesicular RNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44, NAMPT, IL32, TBP, BCL10, ANXA1, TYMP, NFX1;

(iv) CD44, NAMPT, TBP, BCL10, IFNGR1, ANXA1, TYMP, NFX1;

(v) CD44, NAMPT, TBP, BCH10, BMP7, ANXA1, TYMP, NFX1; or

(vi) CD44, NAMPT, TBP, BCL10, STAT1, ANXA1, TYMP, NFX1

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a.) determining the expression level of:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44. NAMPT, IL32, TBP, BCL10, ANXA1, TYMP, NFX1;

(iv) CD44, NAMPT, TBP, BCL10, IFNGR1, ANXA1, TYMP, NFX1;

(v) CD44, NAMPT, TBP, BCL10, BMP7, ANXA1, TYMP, NFX1; or

(vi) CD44, NAMPT, TBP, BCL10, STAT1, ANXA1, TYMP, NFX1

in microvesicular RNA and cell-free DNA isolated from a biological sample from the subject; b) inputting the expression levels from step (a) into an algorithm to generate a score; c) determining the risk of an antibody-mediated kidney transplant rejection in the subject based on the score.

The present disclosure provides a method of identifying antibody-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44, NAMPT, IL32, TBP, BCL10, ANXA1, TYMP, NFX1;

(iv) CD44, NAMPT, TBP, BCL10, IFNGR1, ANXA1, TYMP, NFX1;

(v) CD44, NAMPT, TBP, BCL10,BMP7, ANXA1, TYMP, NFX1; or

(vi) CD44, NAMPT, TBP, BCL10, STAT1, ANXA1, TYMP, NFX1

in microvesicular RNA and cell-free DNA isolated from a biological sample from the subject; h) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

The present disclosure provides a method of determining the risk of an antibody-mediated kidney transplant rejection in a subject who has undergone a kidney transplant, the method comprising: a) determining the expression level of at least one, or at least two, or at least three, or at least four, or at least five, or at least six, or at least seven, or at least eight genes in at least one of the following gene sets:

(i) CD44, NAMPT, PYCARD, TBP, BCL10, ANXA1, TYMP, NFX1;

(ii) CD44, NAMPT, IRAK2, TBP, BCL10, ANXA1, TYMP, NFX1;

(iii) CD44, IL32, TBP, BCL10, ANXA1, TYMP, NFX1;

(iv) CD44, NAMPT, TBP, BCL10, IFNGR1, ANXA1, TYMP, NFX1;

(v) CD44, NAMPT, TBP, BCL10, BMP7, ANXA1, TYMP, NFX1; or

(vi) CD44, NAMPT, TBP, BCL10, STAT1, ANXA1, TYMP, NFX1

in microvesicular RNA and cell-free DNA isolated from a biological sample from the subject; b) inputting the expression levels from step (a) into an algorithm to generate a score; c) determining the risk of an antibody-mediated kidney transplant rejection in the subject based on the score.

In some aspects of the preceding methods, step (a) can comprise determining the expression level of CD44, NAMPT, PYCARD, TBP BCL10, ANXA1, TYMP, NFX1.

In some aspects of the preceding methods, step (a) can comprise determining the expression level of CD44, NAMPT, IRAK2, TBP, BCL10, ANXA1, TYMP, NFX1.

In some aspects of the preceding methods, step (a) can comprise determining the expression level of CD44, NAMPT, IL32, TBP, BCL10, ANXA1, TYMP, NFX1.

In some aspects of the preceding methods, step (a) can comprise determining the expression level of CD44, NAMPT, TBP, BCL10, IFNGR1, ANXA1, TYMP, NFX1.

In some aspects of the preceding methods, step (a) can comprise determining the expression level of CD44, NAMPT, TBP, BCL10, BMP7, ANXA1, TYMP, NFX1.

In some aspects of the preceding methods, step (a) can comprise determining the expression level of CD44, NAMPT, TBP, BCL10, STAT1, ANXA1, TYMP, NFX1.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of at least two of five biomarkers microvesicular RNA isolated from a biological sample from the subject, wherein the five biomarkers comprise CD74, C3, CXCL11, CD44 and IFNAR2; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of at least two of five biomarkers in microvesicular RNA isolated from a biological sample from the subject, wherein the five biomarkers comprise CD74, C3, CXCL11, CD44 and IFNAR2; b) inputting the expression levels from step (a) into an algorithm to generate a score; c) determining the risk of an antibody-mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of at least two of five biomarkers in microvesicular RNA and cell-free DNA isolated from a biological sample from the subject, wherein the five biomarkers comprise CD74, C3, CXCL11, CD44 and IFNAR2; b) inputting the expression levels from step (a) into an algorithm to generate a score; c) comparing the score to a predetermined cutoff value; d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of at least two of five biomarkers in microvesicular RNA and cell-free DNA isolated from a biological sample from the subject, wherein the five biomarkers comprise CD74, C3, CXCL11, CD44 and IFNAR2; b) inputting the expression levels from step (a.) into an algorithm to generate a score; c) determining the risk of an antibody-mediated kidney transplant rejection as opposed to a cell-mediated kidney transplant rejection in the subject based on the score.

In some aspects of the preceding methods, step (a) can comprise determining the expression level of at least three, or at least four of the 5 biomarkers.

In some aspects of the preceding methods, step (a) can comprise determining the expression level of each of the 5 biomarkers.

In some aspects of the preceding methods, step (a) can comprise the subject can be identified as having a kidney transplant rejection using any of the methods described herein.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of:

(i) CD74, C3, CXCL11 and CD44;

(ii) CD74, C3 and CXCL11; or

(iii) CD74 and C3

in microvesicular RNA isolated from a. biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of:

(i) CD74, C3, CXCL11 and CD44;

(ii) CD74, C3 and CXCL11; or

iii) CD74 and C3

in microvesicular RNA isolated from a biological sample from the subject; I)) inputting the expression levels from step (a) into an algorithm to generate a score; c) determining the risk of an antibody-mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of at least one, or at least two, or at least three genes in at least one of the following gene sets:

(i) CD74, C3, CXCL11 and CD44;

(ii) CD74, C3 and CXCL11; or

(iii) CD74 and C3

in microvesicular RNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of at least one, or at least two, or at least three genes in at least one of the following gene sets:

(i) CD74, C3, CXCL11 and CD44;

(ii) CD74, C3 and CXCL11; or

(iii) CD74 and C3

in microvesicular RNA isolated from a biological sample from the subject; b) inputting the expression levels from step (a) into an algorithm to generate a score; c) determining the risk of an antibody-mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of

(i) CD74, C3, CXCL11 and CD44;

(ii) CD74, C3 and CXCL11; or

(iii) CD74 and C3

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a. subject who has undergone a kidney transplant and has been identified as having a kidney transplant rejection, the method comprising: a) determining the expression level of:

(i) CD74, C3, CXCL11 and CD44;

(ii) CD74, C3 and CXCL11; or

(iii) CD74 and C3

in microvesicular RNA and cell-free DNA isolated from a biological sample from the subject; b) inputting the expression levels from step (a) into an algorithm to generate a score; c) determining the risk of an antibody-mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of at least one, or at least two, or at least three, or at least four genes in at least one of the following gene sets:

(i) CD74, C3, CXCL11 and CD44;

(ii) CD74, C3 and CXCL11; or

(iii) CD74 and C3

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of at least one, or at least two, or at least three genes in at least one of the following gene sets:

(i) CD74, C3, CXCL11 and. CD44;

(ii) CD74, C3 and CXCL11; or

(iii) CD74 and C3

in microvesicular RNA and cell-free DNA isolated from a biological sample from the subject; b) inputting the expression levels from step (a) into an algorithm to generate a score; c) determining the risk of an antibody-mediated kidney transplant rejection as opposed to a cell-mediated kidney transplant rejection in the subject based on the score.

In some aspects of the preceding methods, step (a) can comprise determining the expression level of CD74, C3, CXCL11 and CD44.

In some aspects of the preceding methods, step (a) can comprise determining the expression level of CD74, C3 and CXCL11.

In some aspects of the preceding methods, step (a) can comprise determining the expression level of CD74 and C3.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of

(i) C3, CXCL11;

(ii) C3, CD44;

(iii) C3, CXCL11, CD44; or

(iv) CD74, C3, CD44

in microvesicular RNA isolated from a biological sample from the subject; b) inputting the expression levels from step (a) into an algorithm to generate a score; c) comparing the score to a predetermined cutoff value; d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of:

(i) C3, CXCL11;

(ii) C3, CD44;

(iii) C3, CXCL11, CD44; or

(iv) CD74, C3, CD44

in microvesicular RNA isolated from a biological sample from the subject; h) inputting the expression levels from step (a) into an algorithm to generate a score; c) determining the risk of an antibody mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of at least one, or at least two, or at least three genes in at least one of the following gene sets:

(i) C3, CXCL11;

(ii) C3, CD44;

(iii) C3, CDCL11, CD44; or

(iv) CD74, C3, CD44

in microvesicular RNA isolated from a biological sample from the subject; b) inputting the expression levels from step (a) into an algorithm to generate a score; c) comparing the score to a predetermined cutoff value; d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a. cell-mediated kidney transplant rejection in a subject who has undergone a kidney transplant and has been identified as having a kidney transplant rejection, the method comprising: a) determining the expression level of at least one, or at least two, or at least three, or at least four genes in at least one of the following gene sets:

(i) C3, CXCL11;

(ii) C3, CD44;

(iii) C3, CXCL11, CD44; or

(iv) CD74, C3, CD44

in microvesicular RNA isolated from a biological sample from the subject; b) inputting the expression levels from step (a) into an algorithm to generate a score; c) determining the risk of an antibody-mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of

(i) C3, CXCL11;

(ii) C3, CD44;

(iii) C3, CXCL11, CD44; or

(iv) CD74, C3, CD44

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of:

(i) C3, CXCL11;

(ii) C3, CD44;

(iii) C3, CXCL11, CD44; or

(iv) CD74, C3, CD44

in microvesicular RNA and cell-free DNA isolated from a biological sample from the subject; b) inputting the expression levels from step (a) into an algorithm to generate a score; c) determining the risk of an antibody-mediated kidney transplant rejection as opposed to a cell-mediated kidney transplant rejection in the subject based on the score.

The present disclosure provides a method of identifying antibody-mediated kidney transplant rejection or cell-mediated kidney transplant rejection in a subject who has undergone a kidney transplant and has been identified as having a kidney transplant rejection, the method comprising: a) determining the expression level of at least one, or at least two, or at least three genes in at least one of the following gene sets:

(i) C3, CXCL11;

iii) C3, CD44;

(iii) C3, CXCL11, CD44; or

(iv) CD74, C3, CD44

in microvesicular RNA and cell-free DNA isolated from a biological sample from the subject; b) inputting the expression levels from step (a) into an algorithm to generate a score; c) comparing the score to a predetermined cutoff value; and d) identifying antibody-mediated kidney transplant rejection in the subject when the score is greater than or equal to the predetermined cutoff value or identifying the cell-mediated kidney transplant rejection in the subject when the score is less than the predetermined cutoff value.

The present disclosure provides a method of determining the risk of an antibody-mediated kidney transplant rejection as opposed to a cell-mediated kidney transplant rejection in a subject who has undergone a kidney transplant and has been identified as having a kidney transplant rejection, the method comprising: a) determining the expression level of at least one, or at least two, or at least three, or at least four genes in at least one of the following gene sets:

(i) C3, CXCL11;

(ii) C3, CD44;

(iii) C3, CXCL11, CD44; or

(iv) CD74, C3, CD44

in microvesicular RNA and cell-free DNA isolated from a biological sample from the subject; b) inputting the expression levels from step (a) into an algorithm to generate a score; c) determining the risk of an antibody-mediated kidney transplant rejection as opposed to a. cell-mediated kidney transplant rejection in the subject based on the score.

In some aspects of the preceding methods, step (a) can comprise determining the expression level of C3, CXCL11.

In some aspects of the preceding methods, step (a) can comprise determining the expression level of C3, CD44.

In some aspects of the preceding methods, step (a) can comprise determining the expression level of C3, CXCL11, CD44.

In some aspects of the preceding methods, step (a) can. comprise determining the expression level of CD74, C3, CD44.

The present disclosure provides a method of identifying kidney transplant rejection in a subject who has undergone a kidney transplant, the method comprising: a) determining the expression level of at least two of eight biomarkers in microvesicular RNA isolated from a biological sample from the subject, wherein the eight biomarkers comprise TBP, CXCL 10, IFNA4, IL32, UBE2D2, STAT5B, GPI and PYCARD; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of identifying kidney transplant rejection in a subject who has undergone a kidney transplant, the method comprising: a) determining the expression level of at least two of eight biomarkers in microvesicular RNA and cell-free DNA isolated from a biological sample from the subject, wherein the eight biomarkers comprise TBP, CXCL10, IFNA4, IL32, UBE2D2, STAT5B, GPI and PYCARD; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of treating kidney transplant rejection in a. subject who has undergone a kidney transplant, the method comprising: a) determining the expression level of at least two of eight biomarkers in microvesicular RNA isolated from a biological sample from the subject, wherein the eight biomarkers comprise TBP, CXCL10, IFNA4, IL32, UBE2D2, STAT5B, GPI and PYCARD; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

The present disclosure provides a method of treating kidney transplant rejection in a subject who has undergone a kidney transplant, the method comprising: a) determining the expression level of at least two of eight biomarkers in microvesicular RNA and cell-free DNA isolated from a biological sample from the subject, wherein the eight biomarkers comprise TBP, CXCL10, IFNA4, IL32, UBE2D2, STAT5B, GPI and PYCARD; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

In some aspects of the preceding methods, the kidney transplant rejection can be any-cause kidney transplant rejection.

In some aspects of the preceding methods, step (a) can comprise determining the expression level of at least three of the eight biomarkers, or at least four of the eight biomarkers, or at least five of the eight biomarkers, or at least six of the eight biomarkers, or at least seven of the eight biomarkers. In some aspects of the preceding methods, step (a) can comprise determining the expression level of each of the eight biomarkers.

The present disclosure provides a method of identifying kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CXCR4, CD74, HPRT1, CXCL10, TLR10, IFNA4, UBE2D2, GPI, F3, IFNE, FPR2, CXCR2 and IL32; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of identifying kidney transplant rejection in a subject who has undergone a kidney transplant, the method comprising: a) determining the expression level of at least two of 13 biomarkers in microvesicular RNA and cell-free DNA isolated from a biological sample from the subject, wherein the 13 biomarkers comprise CXCR4, CD74, HPRT1, CXCL10, TLR10, IFNA4, UBE2D2, GPI, F3, IFNE, FPR2, CXCR2 and IL32; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of treating kidney transplant rejection in a subject who has undergone a kidney transplant, the method comprising: a) determining the expression level of at least two of 13 biomarkers in microvesicular RNA isolated from a biological sample from the subject, wherein the 13 biomarkers comprise CXCR4, CD74, HPRT1, CXCL10, TLR10, IFNA4, UBE2D2, GPI, F3, IFNE, FPR2, CXCR2 and IL32; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

The present disclosure provides a method of treating kidney transplant rejection in a subject who has undergone a kidney transplant, the method comprising: a) determining the expression level of at least two of 13 biomarkers in microvesicular RNA and cell-free DNA isolated from a biological sample from the subject, wherein the 13 biomarkers comprise CXCR4, CD74, HPRT1, CXCL10, TLR10, IFNA4, UBE2D2, GPI, F3, IFNE, FPR2, CXCR2 and IL32; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

In some aspects of the preceding methods, the kidney transplant rejection can be a cell-mediated kidney transplant rejection. The cell-mediated kidney transplant rejection can be T-cell-mediated rejection (TCMR).

In some aspects of the preceding methods, step (a) can comprise determining the expression level of at least three of the 13 biomarkers, or at least four of the 13 biomarkers, or at least five of the 13 biomarkers, or at least six of the 13 biomarkers, or at least seven of the 13 biomarkers, or at least eight of the 13 biomarkers, or at least nine of the 13 biomarkers, or at least 10 of the 13 biomarkers, or at least 11 of the 13 biomarkers, or at least 12 of the 13 biomarkers, In some aspects of the preceding methods, step (a) can comprise determining the expression level of each of the 13 biomarkers.

The present disclosure provides a method of identifying kidney transplant rejection in a subject who has undergone a kidney transplant, the method comprising: a) determining the expression level of at least two of 10 biomarkers in microvesicular RNA isolated from a biological sample from the subject, wherein the 10 biomarkers comprise CXCL10, IL32, UBE2D2, F3, TBP, NAMPT, CD74, IFNA4, PYCARD and IFNGR1; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of identifying kidney transplant rejection in a subject who has undergone a kidney transplant, the method comprising: a) determining the expression level of at least two of 10 biomarkers in microvesicular RNA and cell-free DNA isolated from a biological sample from the subject, wherein the 10 biomarkers comprise CXCL10, IL32, UBE2D2, F3, TBP, NAMPT, CD74, IFNA4, PYCARD and IFNGR1; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of treating kidney transplant rejection in a subject who has undergone a kidney transplant, the method comprising: a) determining the expression level of at least two of 10 biomarkers in microvesicular RNA isolated from a biological sample from the subject, wherein the 10 biomarkers comprise CXCL10, IL32, UBE2D2, F3, TBP, NAMPT, CD74, IFNA4, PYCARD and IFNGR1; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

The present disclosure provides a method of treating kidney transplant rejection in a. subject who has undergone a kidney transplant, the method comprising: a) determining the expression level of at least two of 10 biomarkers in microvesicular RNA and cell-free DNA isolated from a biological sample from the subject, wherein the 10 biomarkers comprise CXCL,10, IL32, UBE2D2, F3, TBP, NAMPT, CD74, IFNA4, PYCARD and IFNGR1; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

In some aspects of the preceding methods, the kidney transplant rejection can be any-cause kidney transplant rejection.

In some aspects of the preceding methods, step (a) can comprise determining the expression level of at least three of the 10 biomarkers, or at least tour of the 10 biomarkers, or at least five of the 10 biomarkers, or at least six of the 10 biomarkers, or at least seven of the 10 biomarkers, or at least eight of the 10 biomarkers, or at least nine of the 10 biomarkers. In some aspects of the preceding methods, step (a) can comprise determining the expression level of each of the 10 biomarkers,

The present disclosure provides a method of identifying kidney transplant rejection in a subject who has undergone a kidney transplant, the method comprising: a) determining the expression level of at least two of five biomarkers in microvesicular RNA isolated from a. biological sample from the subject, wherein the five biomarkers comprise F3, CD74, CXCL10, UBE2D2 and IFNA4; h) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of identifying kidney transplant rejection in a subject who has undergone a kidney transplant, the method comprising: a) determining the expression level of at least two of five biomarkers in microvesicular RNA and cell-free DNA isolated from a. biological sample from the subject, wherein the five biomarkers comprise F3, CD74, CXCL10, UBE2D2 and IFNA4; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of treating kidney transplant rejection in a subject who has undergone a kidney transplant, the method comprising: a) determining the expression level of at least two of five biomarkers microvesicular RNA isolated from a biological sample from the subject, wherein the five biomarkers comprise F3, CD74, CXCL10, UBE2D2 and IFNA4; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

The present disclosure provides a method of treating kidney transplant rejection in a subject who has undergone a kidney transplant, the method comprising: a) determining the expression level of at least two of five biomarkers in microvesicular RNA and cell-free DNA isolated from a biological sample from the subject, wherein the five biomarkers comprise F3, CD74, CXCL10, UBE2D2 and IFNA4; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

In some aspects of the preceding methods, the kidney transplant rejection can be cell-mediated kidney transplant rejection. Cell-mediated kidney transplant rejection can be T-cell-mediated rejection (TCMR).

In some aspects of the preceding methods, step (a) can comprise determining the expression level of at least three of the five biomarkers or at least four of the five biomarkers. In some aspects of the preceding methods, step (a) can comprise determining the expression level of each of the five biomarkers.

The present disclosure provides a method of identifying kidney transplant rejection in a subject who has undergone a kidney transplant, the method comprising: a) determining the expression level of at least two of five biomarkers in microvesicular RNA isolated from a biological sample from the subject, wherein the five biomarkers comprise HPRT1, CXCR4, CXCL10, IL32 and IFNA4; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of identifying kidney transplant rejection in a subject who has undergone a kidney transplant, the method comprising: a) determining the expression level of at least two of five biomarkers in microvesicular RNA and cell-free DNA isolated from a biological sample from the subject, wherein the five biomarkers comprise HPRT1, CXCR4, CXCL10, IL32 and IFNA4; b) inputting the expression levels from step (a) into an algorithm to generate a score; c) comparing the score to a predetermined cutoff value; d) identifying kidney transplant rejection in the subject when the score is greater than or equal to the predetermined cutoff value or identifying the lack of kidney transplant rejection in the subject when the score is less than the predetermined cutoff value.

The present disclosure provides a method of treating kidney transplant rejection in a subject who has undergone a kidney transplant, the method comprising: a) determining the expression level of at least two of five biomarkers in microvesicular RNA isolated from a biological sample from the subject, wherein the five biomarkers comprise HPRT1, CXCR4, CXCL10, IL32 and IFNA4; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

The present disclosure provides a method of treating kidney transplant rejection in a subject who has undergone a kidney transplant, the method comprising: a) determining the expression level of at least two of five biomarkers in microvesicular RNA and cell-free DNA isolated from a biological sample from the subject, wherein the five biomarkers comprise HPRT1, CXCR4, CXCL10, IL32 and IFNA4; b) inputting the expression levels from step (a) into an algorithm to generate a score; c) comparing the score to a predetermined cutoff value; d) administering at least one kidney transplant rejection therapy to the subject when the score is greater than or equal to the predetermined cutoff value.

In some aspects of the preceding methods, the kidney transplant rejection can be antibody-mediated kidney transplant rejection.

In some aspects of the preceding methods, step (a) can comprise determining the expression level of at least three of the five biomarkers or at least four of the five biomarkers. In some aspects of the preceding methods, step (a) can comprise determining the expression level of each of the five biomarkers.

In some aspects, any method of the present disclosure, prior to step (a). can further comprise: i) isolating a plurality of microvesicles from a biological sample from the subject; and ii) extracting at least one microvesicular RNA from the plurality of isolated microvesicles.

In some aspects, any method of the present disclosure, prior to step (a), can further comprise: i) isolating a microvesicle fraction from a biological sample from the subject, wherein the microvesicle fraction comprises a plurality of microvesicles and cfDNA; ii) extracting at least one microvesicular RNA and at least one cfDNA molecule from the plurality of isolated microvesicles.

In some aspects of the methods of the present disclosure, isolating a plurality of microvesicles from a biological sample from the subject can comprise a processing step to remove cells, cellular debris or a combination of cells and cellular debris. A processing step can comprise filtering the sample, centrifuging the sample, or a combination of filtering the sample and centrifuging the sample. Centrifuging can comprise centrifuging at about 2000×g. Filtering can comprise filtering the sample through a filter with a pore size of about 0.8 microns.

In some aspects of the methods of the present disclosure, isolating a plurality of microvesicles can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof

In some aspects of the methods of the present disclosure, isolating a microvesicle fraction, wherein the microvesicle fraction comprises a plurality of microvesicles and cfDNA can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating an at least one microvesicle is from a bodily fluid sample can comprise contacting the bodily fluid sample with at least one affinity agent that binds to at least one surface marker present on the surface the at least one microvesicle.

Other microvesicle and microvesicle fraction isolation procedures are described in US 2017-0088898 A1, US 2016-0348095 A1, US 2016-0237422 A1, US 2015-0353920 A1, U.S. Pat. No. 10,465,183 and US 2019-0284548 A1, the contents of each of which are incorporated herein by reference in their entireties. The methods of the present disclosure can comprise any of the methods described in the aforementioned United States Patent Publications and United States Patents.

In some aspects of the methods of the present disclosure, a biological sample can be a urine sample, a first-catch urine sample or a second voided urine sample. A biological sample can have a volume of between at least about 1 ml to at least about 50 ml, A biological sample can have a volume of up to about 20 ml. A biological sample can have a volume of at least 3 ml.

In some aspects of the methods of the present disclosure, step (a) can further comprise: (i) determining the expression level of at least one reference biomarker; (ii) normalizing the expression level of the at least two, or the at least three, or the at least four, of the at least five, or at the at least six, or the at least seven, or the at least eight, or the at least nine, or the at least 10, or the at least 11, or the at least 12, or the at least 13 biomarkers to the expression level of the at least one reference biomarker. In some aspects of the methods of the present disclosure inputting the expression levels from step (a) into an algorithm to generate a score can comprise inputting the normalized expression levels from step (a) into an algorithm to generate a score. In some aspects, an at least one reference biomarker can comprise PGK1.

In some aspects of the methods of the present disclosure, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis or any combination thereof

In some aspects of the methods of the present disclosure, a predetermined cutoff value can have, or be selected as to have, a negative predictive value (NPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can have, or be selected as to have, a positive predictive value (PPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%. or at least about 95%, or at least about 99%, or at least about 99.9%.

In some aspects of the methods of the present disclosure, a predetermined cutoff value can have, or be selected as to have, a sensitivity of at least about 10%, or at least about 15%, or at least about 20%. or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%,

In some aspects of the methods of the present disclosure, a predetermined cutoff value can have, or be selected as to have, a specificity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected as to be optimized to rule-out kidney transplant rejection. Without wishing to he bound by theory, such a predetermined cutoff value would he advantageous in situations where kidney transplant rejection has been clinically indicated (e.g, serum creatinine levels in a subject are rising).

In some aspects of the methods of the present disclosure, a predetermined cutoff value can be selected as to be optimized to rule-in kidney transplant rejection. In a non-limiting example, such a predetermined cutoff value could have a high positive predictive value. Without wishing to be bound by theory, such a predetermined cutoff value would be advantageous in situations where kidney transplant rejection has not been clinically indicated and/or a clinician is determining whether to proceed with renal biopsy and/or kidney transplant rejection therapy.

In some aspects of the methods of the present disclosure, a predetermined cutoff value can be calculated and/or selected using at least one receiver operating characteristic (ROC) curve. In some aspects of the methods of the present disclosure, a predetermined cutoff value can be calculated and/or selected to have any of the features described herein (e.g, a specific sensitivity, specificity, PPV, NPV or any combination thereof) using any method known in the art, as would be appreciated by the skilled artisan.

In some aspects of the methods of the present disclosure, an algorithm can be the product of a feature selection wrapper algorithm. In some aspects of the methods of the present disclosure, an algorithm can be the product of a machine learning algorithm. in some aspects of the methods of the present disclosure, an algorithm can be the product of a trained classifier built from at least one predictive classification algorithm. In some aspects of the methods of the present disclosure, an algorithm can be the product of a of a logistic regression model. A logistic regression model can comprise LASSO regularization. In some aspects, an algorithm can be the product of a feature selection wrapper algorithm, a machine learning algorithm, a trained classifier built from at least one predictive classification algorithm or any combination thereof. In some aspects of the methods of the present disclosure, an algorithm can be the product of a feature selection wrapper algorithm and a trained classifier built from at least one predictive classification algorithm.

In some aspects of the methods of the present disclosure a predictive classification algorithm, a feature selection wrapper algorithm, and/or a machine learning algorithm can comprise XGBoost (XGB), random forest (RF), Lasso and Elastic-Net Regularized Generalized Linear Models (glmnet), cforest, classification and regression tree (CART), treebag, k nearest-neighbor (knn), neural network (nnet), support vector machine-radial (SVM-radial), support vector machine-linear (SVM-linear), naïve bayes (NB), multilayer perceptron (trip), Boruta (see Kursa M B, Rudnicki W R. Feature Selection with the Boruta Package. J Stat Softw 2010; 36(11), incorporated herein by reference in its entirety) or any combination thereof.

In some aspects of the methods of the present disclosure, an algorithm can be the product of a feature selection wrapper algorithm and a trained classifier built from at least one predictive classification algorithm. The feature selection wrapper algorithm can be Boruta and the at least one predicative classification algorithm can be SVM-radial.

In some aspects of the methods of the present disclosure, an algorithm can a product of a feature selection wrapper algorithm, machine learning algorithm, trained classifier, logistic regression model or any combination thereof, that was trained to identify kidney transplant rejection in a subject using: a) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is kidney transplant rejection negative: and b) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is kidney transplant rejection positive. In some aspects, the at least one subject who is kidney transplant rejection negative is determined to be kidney transplant rejection negative based on kidney transplant biopsy results. In some aspects, the at least one subject who is kidney transplant rejection positive is determined to be kidney transplant rejection positive based on kidney transplant biopsy results.

In some aspects of the methods of the present disclosure, an algorithm can a product of a feature selection wrapper algorithm, machine learning algorithm, trained classifier, logistic regression model or any combination thereof, that was trained to identify cell-mediated kidney transplant rejection in a subject using: a) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is cell-mediated kidney transplant rejection negative; and b) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is cell-mediated kidney transplant rejection positive. In some aspects, the at least one subject who is cell-mediated kidney transplant rejection negative is determined to be cell-mediated kidney transplant rejection negative based on kidney transplant biopsy results. In some aspects, the at least one subject who is cell-mediated kidney transplant rejection positive is determined to be cell-mediated kidney transplant rejection positive based on kidney transplant biopsy results.

In some aspects of the methods of the present disclosure, an algorithm can a product of a feature selection wrapper algorithm, machine learning algorithm, trained classifier, logistic regression model or any combination thereof, that was trained to identify antibody-mediated kidney transplant rejection in a subject using: a) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is antibody-mediated kidney transplant rejection negative; and b) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is antibody-mediated kidney transplant rejection positive. In some aspects, the at least one subject who is antibody-mediated kidney transplant rejection negative is determined to be antibody-mediated kidney transplant rejection negative based on kidney transplant biopsy results. In some aspects, the at least one subject who is antibody-mediated kidney transplant rejection positive is determined to be antibody-mediated kidney transplant rejection positive based on kidney transplant biopsy results.

In some aspects of the methods of the present disclosure, an algorithm can a product of a feature selection wrapper algorithm, machine learning algorithm, trained classifier, logistic regression model or any combination thereof, that was trained to identify antibody-mediated kidney transplant rejection in a subject as opposed to cell-mediated kidney transplant rejection using: a) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is antibody-mediated kidney transplant rejection positive; and b) the expression levels of the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15 biomarkers in at least one biological sample from at least one subject who is cell-mediated kidney transplant rejection positive. In some aspects, the at least one subject who is antibody-mediated kidney transplant rejection positive is determined to be antibody-mediated kidney transplant rejection positive based on kidney transplant biopsy results. In some aspects, the at least one subject who is cell-mediated kidney transplant rejection positive is determined to be cell-mediated kidney transplant rejection positive based on kidney transplant biopsy results.

The methods of the present disclosure can further comprise administering at least one kidney transplant rejection therapy to a subject identified as having kidney transplant rejection. The methods of the present disclosure can further comprise administering at least one kidney transplant rejection therapy to a subject identified as having a high risk of having a kidney transplant rejection.

The methods of the present disclosure can further comprise administering at least one kidney transplant rejection therapy to a subject identified as having kidney transplant rejection, wherein the subject does not require a renal biopsy.

In some aspects of the methods of the present disclosure, administering at least one kidney transplant rejection therapy can comprise administering an increased amount of a kidney transplant rejection therapy that the subject was previously receiving. In some aspects of the methods of the present disclosure, administering at least one kidney transplant rejection therapy can comprise augmenting or supplementing a kidney transplant rejection therapy that the subject was previously receiving.

In some aspects of the methods of the present disclosure, an at least one kidney transplant rejection therapy can comprise administering to the subject at least one therapeutically effective amount of at least one immunosuppressant, at least one therapeutically effective amount of at least one steroid, at least one therapeutically effective amount of at least one corticosteroid, at least one therapeutically effective amount of at least one steroid, at least one therapeutically effective amount of at least one anti-T-cell antibody, at least one therapeutically effective amount of mycophenolate mofetil (MMF), at least one therapeutically effective amount of cyclosporine A (CsA), at least one therapeutically effective amount of tacrolimus, at least one therapeutically effective amount of azathioprine, at least one therapeutically effective amount of muromonab (OKT-3), at least one therapeutically effective amount of anti-thymocyte globulin (AIG), at least one therapeutically effective amount of anti-lymphocyte globulin (ALG), at least one therapeutically effective amount of Campath (alemtuzwnab), at least one therapeutically effective amount of prednisone, at least one therapeutically effective amount of mycophenolic acid, at least one therapeutically effective amount of rapamycin, at least one therapeutically effective amount of belatacept, at least one therapeutically effective amount of intravenous immunoglobulin (IVIg), at least one therapeutically effective amount of an anti-CD20 agent (e.g. rituximab), at least one therapeutically effective amount of bortezomib or any combination thereof.

In some aspects, an at least one kidney transplant rejection therapy can comprise performing plasmapheresis.

In some aspects, a therapeutically effective amount of at least one steroid comprises a high dose regimen of the at least one steroid.

In some aspects, a therapeutically effective amount of at least one corticosteroid comprises a high dose regimen of the at least one steroid.

The methods of the present disclosure can further comprise administering at least one cell-mediated kidney transplant rejection therapy to a subject identified as having cell-mediated kidney transplant rejection. The methods of the present disclosure can further comprise administering at least one cell-mediated kidney transplant rejection therapy to a subject identified as having a high risk of having cell-mediated kidney transplant rejection.

In some aspects, a cell-mediated kidney transplant rejection therapy can comprise administering to the subject at least one therapeutically effective amount of at least one steroid, at least one therapeutically effective amount of at least one corticosteroid, at least one therapeutically effective amount of muromonab (OKT-3), at least one therapeutically effective amount of anti-thymocyte globulin (ATG), at least one therapeutically effective amount of Campath (alemtuzurnab), at least one therapeutically effective amount of prednisone, at least one therapeutically effective amount of tacrolimus, at least one therapeutically effective amount of cyclosporine A, at least one therapeutically effective amount of mycophenolic acid, at least one therapeutically effective amount of azathioprine, at least one therapeutically effective amount of rapamycin, at least one therapeutically effective amount of belatacept, or any combination thereof.

In some aspects, a cell-mediated kidney transplant rejection therapy can comprise administering to the subject at least one therapeutically effective amount of at least one steroid, at least one therapeutically effective amount of at least one corticosteroid, at least one therapeutically effective amount of murornonab (OKT-3), at least one therapeutically effective amount of anti-thymocyte globulin (ATG), at least one therapeutically effective amount of Campath (alemtuzumab), or any combination thereof.

The methods of the present disclosure can further comprise optimizing existing maintenance therapy that a subject is undergoing when the subject is identified as having cell-mediated kidney transplant rejection. The methods of the present disclosure can further comprise optimizing existing maintenance therapy that a subject is undergoing when the subject is identified as having a high risk of cell-mediated kidney transplant rejection. in some aspects, the maintenance therapy can comprise the administration of prednisone, tacrolimus, cyclosporine A, mycophenolic acid, azathioprine, rapamycin, belatacept or any combination thereof.

The methods of the present disclosure can further comprise administering at least one antibody-mediated kidney transplant rejection therapy to a subject identified as having antibody-mediated kidney transplant rejection. The methods of the present disclosure can further comprise administering at least one antibody-mediated kidney transplant rejection therapy to a subject identified as having a high risk of having antibody-mediated kidney transplant rejection.

In some aspects, an antibody-mediated kidney transplant rejection therapy can comprise administering to the subject at least one therapeutically effective amount of at least one steroid, at least one therapeutically effective amount of at least one corticosteroid, at least one therapeutically effective amount of anti-thymocyte globulin (ATG), at least one therapeutically effective amount of intravenous immunoglobulin (IVIg), at least one therapeutically effective amount of an anti-CD20 agent (e.g. rituxitnab), at least one therapeutically effective amount of bortezomib, or any combination thereof.

In some aspects, determining the risk of a kidney transplant rejection in a subject can comprise determining that the subject is at a high risk of having a kidney transplant rejection. In some aspects, determining the risk of a kidney transplant rejection in a subject can comprise determining that the subject is at a low risk of having a kidney transplant rejection. In some aspects, the methods of the present disclosure can further comprise administering at least one kidney transplant rejection therapy to a subject identified as having a high risk of kidney transplant rejection.

In some aspects, determining the risk of a kidney transplant rejection in a subject based on a score can comprise: i) comparing the score to a predetermined cutoff value; and ii) determining that the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, determining the risk of an antibody-mediated kidney transplant rejection in a subject can comprise determining that the subject is at a high risk of having an antibody-mediated kidney transplant rejection. In some aspects, determining the risk of an antibody-mediated kidney transplant rejection in a subject can comprise determining that the subject is at a low risk of having an antibody-mediated kidney transplant rejection. In some aspects, the methods of the present disclosure can further comprise administering at least one kidney transplant rejection therapy to a subject identified as having a high risk of an antibody-mediated kidney transplant rejection.

In some aspects, determining the risk of an antibody-mediated kidney transplant rejection in a subject based on a score can comprise: i) comparing the score to a predetermined cutoff value; and ii) determining that the subject is at a high risk of having an antibody-mediated kidney transplant rejection when the score is greater than or equal to the predetermined cutoff value or determining that the subject is at low risk of having an antibody-mediated kidney transplant rejection when the score is less than the predetermined cutoff value.

In some aspects, determining the risk of a cell-mediated kidney transplant rejection in a subject can comprise determining that the subject is at a high risk of having a cell-mediated kidney transplant rejection. In some aspects, determining the risk of a cell-mediated kidney transplant rejection in a subject can comprise determining that the subject is at a low risk of having a cell-mediated kidney transplant rejection. In some aspects, the methods of the present disclosure can further comprise administering at least one kidney transplant rejection therapy to a subject identified as having a high risk of a cell-mediated kidney transplant rejection.

In some aspects, determining the risk of a cell-mediated kidney transplant rejection in a subject based on a score can comprise: i) comparing the score to a predetermined cutoff value; and ii) determining that the subject is at a high risk of having a cell-mediated kidney transplant rejection when the score is greater than or equal to the predetermined cutoff value or determining that the subject is at low risk of having a cell-mediated kidney transplant rejection when the score is less than the predetermined cutoff value,

In some aspects, determining the risk of an antibody-mediated kidney transplant rejection as opposed to a cell-mediated kidney transplant rejection in a subject based on a score can comprise: comparing the score to a predetermined cutoff value; and ii) determining that the subject is at a higher risk of having an antibody-mediated kidney transplant rejection as opposed to a cell-mediated kidney transplant rejection when the score is greater than or equal to the predetermined cutoff value, or determining that the subject is at a higher risk of having a cell-mediated kidney transplant rejection when the score is less than the predetermined cutoff value.

In some aspects, determining the risk of an antibody-mediated kidney transplant rejection as opposed to a cell-mediated kidney transplant rejection in a subject based on a score can comprise i) comparing the score to a predetermined cutoff value; and ii) determining that the subject is at a higher risk of having a cell-mediated kidney transplant rejection as opposed to a cell-mediated kidney transplant rejection when the score is greater than or equal to the predetermined cutoff value, or determining that the subject is at a higher risk of having an antibody-mediated kidney transplant rejection when the score is less than the predetermined cutoff value.

In some aspects of the methods of the present disclosure, wherein the method is directed towards: a) identifying antibody-mediated kidney transplant rejection or cell-mediated kidney transplant rejection in a subject who has undergone a kidney transplant and has been identified as having a. kidney transplant rejection; and/or b) determining the risk of an antibody-mediated kidney transplant rejection as opposed to a cell-mediated kidney transplant rejection in a subject who has undergone a kidney transplant and has been identified as having a kidney transplant rejection, the subject can have been identified as having a kidney transplant rejection using at least one of the methods described herein. That is, any one of the methods described herein may be combined with any other method described herein.

In some aspects of the methods of the present disclosure, wherein the method is directed towards: a) identifying antibody-mediated kidney transplant rejection or cell-mediated kidney transplant rejection in a subject who has undergone a kidney transplant and has been identified as having a kidney transplant rejection; and/or b) determining the risk of an antibody-mediated kidney transplant rejection as opposed to a cell-mediated kidney transplant rejection in a subject who has undergone a kidney transplant and has been identified as having a kidney transplant rejection, the subject who has been identified as having a kidney transplant rejection can be a subject that has been identified as having a high risk of a kidney transplant rejection using at least one of the methods described herein. That is, any one of the methods described herein may be combined with any other method described herein.

### Exemplary Embodiments

Embodiment 1. A method of identifying kidney transplant rejection in a subject who has undergone a kidney transplant, the method comprising:

a) determining the expression level of at least two of 15 biomarkers in microvesicular RNA isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP;

b) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 2. A method of identifying kidney transplant rejection in a subject who has undergone a kidney transplant, the method comprising:

a) determining the expression level of at least two of 15 biomarkers in microvesicular RNA and cell-free DNA (cfDNA) isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP;

b) inputting the expression levels from step (a) into an algorithm to generate a score; c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 3. The method of any one of the preceding embodiments, wherein the kidney transplant rejection is any-cause kidney transplant rejection.

Embodiment 4. The method of any one of the preceding embodiments, wherein step (a) comprises determining the expression level of at least three of the 15 biomarkers.

Embodiment 5. The method of any one of the preceding embodiments, Wherein step (a) comprises determining the expression level of at least four of the 1.5 biomarkers.

Embodiment 6. The method of any one of the preceding embodiments, wherein step (a) comprises determining the expression level of at least five of the 15 biomarkers.

Embodiment 7. The method of any one of the preceding embodiments, wherein step (a) comprises determining the expression level of at least six of the 15 biomarkers.

Embodiment 8. The method of any of the preceding embodiments, wherein step (a) comprises determining the expression level of at least seven of the 15 biomarkers.

Embodiment 9. The method of any one of the preceding embodiments, wherein step (a) comprises determining the expression level of at least eight of the 15 biomarkers.

Embodiment 10. The method of any one of the preceding embodiments, wherein step (a) comprises determining the expression level of at least nine of the 15 biomarkers.

Embodiment 11. The method of any one of the preceding embodiments, wherein step (a) comprises determining the expression level of at least ten of the 15 biomarkers.

Embodiment 12. The method of any one of the preceding embodiments, wherein step (a) comprises determining the expression level of at least 11 of the 1.5 biomarkers.

Embodiment 13. The method of any one of the preceding embodiments, wherein step (a) comprises determining the expression level of at least 12 of the 15 biomarkers.

Embodiment 14. The method of any one of the preceding embodiments, wherein step (a) comprises determining the expression level of at least 13 of the 15 biomarkers.

Embodiment 15. The method of any one of the preceding embodiments, wherein step (a) comprises determining the expression level of at least 14 of the 15 biomarkers.

Embodiment 16. The method of any one of the preceding embodiments, wherein step (a) comprises determining the expression level of each of the 15 biomarkers.

Embodiment 17. A method of identifying kidney transplant rejection in a subject who has undergone a kidney transplant, the method comprising:

a) determining the expression level of:
(i) CXCL11, CD74, IL32, STAT1, CXCL14, SERPINA1, B21\4, C3 and PYCARD;
(ii) CXCL11, CD74, IL32, STAT1, CXCL14, SERPINA1, B21\4 and C3;
(iii) CXCL11, CD74, IL32, STAT1, CXCL14, SERPINA1 and B21\4;
(iv) CXCI.11, CD74, IL32, STAT1, CXCL14 and SERPINAI;
(v) CXCL11. CD74, It32, STAT1 and CXCLI4;
(vi) CXCL11 CD74, IL32 and STAT1;
(vii) CXCL11, CD74, and IL32; or
(viii) CXCL11 and CD74
in microvesicular RNA isolated from a biological sample from the subject;

b) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 18. A method of identifying kidney transplant rejection in a subject who has undergone a kidney transplant, the method comprising:

a) determining the expression level of:
(i) CXCL11, CD74, IL32, STAT1, CXCL14, SERPINA1, B2M, C3 and PYCARD;
(ii) CXCL11, CD74, IL32, STAT1, CXCL14, SERPINA1, B2M and C3;
(iii) CXCL11, CD74, IL32, STAT1, CXCL14, SERPINA1 and B2M;
(iv) CXCL11, CD74, IL32, STAT1, CXCL14 and SERPINA1;
(v) CXCL11, CD74, IL32, STAT1 and CXCL14;
(vi) CXCL11, CD74, IL32 and STAT1;
(vii) CXCL11, CD74, and IL32; or
(viii) CXCL11 and CD74
in microvesicular RNA and cell-free DNA (cfDNA) isolated from a biological sample from the subject;

b) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 19. The method of any one of the preceding embodiments, wherein step (a) comprises determining the expression level of CDCL11, CD74, IL32, STAT1, CXCL14, SERPINA1, B2M, C3 and PYCARD.

Embodiment 20. The method of any one of the preceding embodiments, wherein step (a) comprises determining the expression level of CXCL11, CD74, IL32, STAT1, CXCL14, SERPINA1, B2M and C3.

Embodiment 21. The method of any one of the preceding embodiments, wherein step (a) comprises determining the expression level of CXCL11, CD74, IL32, STAT1, CXCL14, SERPINA1 and B2M.

Embodiment 22. The method of any one of the preceding embodiments, wherein step (a) comprises determining the expression level of CXCL11, CD74, IL32, STAT1, CXCL14 and SERPINA1.

Embodiment 23. The method of any one of the preceding embodiments, wherein step (a) comprises determining the expression level of CXCL11, CD74, STAT1 and CXCL14.

Embodiment 24. The method of any one of the preceding embodiments, wherein step (a) comprises determining the expression level of CXCL11, CD74, IL32 and STAT1.

Embodiment 25. The method of any one of the preceding embodiments, wherein step (a) comprises determining the expression level of CXCL11, CD74 and IL32.

Embodiment 26. The method of any one of the preceding embodiments, wherein step (a) comprises determining the expression level of CXCL11 and CD74.

Embodiment 27. A method of identifying kidney transplant rejection in a subject who has undergone a. kidney transplant, the method comprising:

a) determining the expression level of:
(i) CXCL11, STAT1, CXCL14, C3, PYCARD, BMP7, IFNGR1, IRAK2;
(ii) CD74, STAT1, CXCL14, C3, PYCARD, BMP7, IFNGR1, IRAK2;
(iii) IL32, STAT1, CXCL14, C3, PYCARD, BMP7, IFNGR1, IRAK2;
(iv) CXCL11, CD74, IL32, STAT1, CXCL14, C3, PYCARD, BMP7, IFNGR1, IRAK2;
(v) CXCL11, CD74, STAT1, CXCL14, C3, PYCARD, BMP7, IFNGR1, IRAK2;
(vi) CD74, IL32, STAT1, CXCL14, C3, PYCARD, BMP7, IFNGR1, IRAK2; or
(vii) CXCL 11, IL32, STAT1, CXCL14, C3, PYCARD, BMP7, IRAK2
in microvesicular RNA isolated from a biological sample from the subject;

b) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 28, A method of identifying kidney transplant rejection in a subject who has undergone a kidney transplant, the method comprising:

a) determining the expression level of:
(i) CXCL11, STAT1, CXCL14, C3, PYCARD, BMP7, IFNGR1, IRAK2;
(ii) CD74, STAT1, CXCL14, C3, PYCARD, BMP7, IFNGR1, IRAK2;
(iii) IL32, STAT1, CXCL14, C3, PYCARD, BMP7, IFNGR1, IRAK2;
(iv) CXCL 11, CD74, IL32, STAT1, CXCL14, C3, PYCARD, BMP7, IRAK2;
(v) CXCL11, CD74, STAT1, CXCL14, C3, PYCARD, BMP7, IFNGR1, IRAK2;
(vi) CD74, IL32, STAT1, CXCL14, C3, PYCARD, BMP7, IFNGR1, IRAK2; or
(vii) CXCL11, IL32, STAT1, CXCL14, C3, PYCARD, BMP7, IFNGR1, IRAK2
in microvesicular RNA and cell-free DNA (cfDNA) isolated from a biological sample from the subject;

b) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 29, The method of any one of the preceding embodiments, wherein step (a) comprises determining the expression. level of CXCL11, STAT1, CXCL14, C3, PYCARD, BMP7, IFNGR1, IRAK2.

Embodiment 30. The method of any one of the preceding embodiments, wherein step (a) comprises determining the expression level of CD74, STAT1, CXCL14, C3, PYCARD, BMP7, IFNGR1, IRAK2.

Embodiment 31. The method of any one of the preceding embodiments, wherein step (a) comprises determining the expression level of IL32, STAT1, CXCL14, C3, PYCARD, BMP7, IFNGR1, IRAK2.

Embodiment 32. The method of any one of the preceding embodiments, wherein step (a) comprises determining the expression level of CXCL11, CD74, IL32, STAT1, CXCL14, C3, PYCARD, BMP7, IFNGR1, IRAK2.

Embodiment 33. The method of any one of the preceding embodiments, wherein step (a) comprises determining the expression level of CXCL11, CD74, STAT1, CXCL14, C3, PYCARD, BMP7, IFNGR1, IRAK2.

Embodiment 34. The method of any one of the preceding embodiments, wherein step (a) comprises determining the expression level of CD74, IL32, STAT1, CXCL14, C3, PYCARD, BMP7, IFNGR1, IRAK2.

Embodiment 35. The method of any one of the preceding embodiments, wherein step (a) comprises determining the expression level of CXCL11, STAT1, CXCL14, C3, PYCARD, BMP7, IFNGR1, IRAK2.

Embodiment 36. A method of identifying kidney transplant rejection in a subject who has undergone a kidney transplant, the method comprising:

a) determining the expression level of:
(i) CXCL11, CD74, IL32, STAT1, SERPINA1, B2M, IBP, NAMPT, IL18BP;
(ii) CXCL11, CD74, IL32, CXCL14, SERPINA1, B2M, TBP, NAMPT, IL18BP;
(iii) CXCL11, CD74, IL32, SERPINA1, B2M, C3, TBP, NAMPT, IL18BP;
(iv) CXCL11, CD74, IL32, SERPINA1, B2M, PYCARD, TBP, NAMPT, IL18BP;
(v) CXCL11, CD74, IL32, SERPINA1, B2M, BMP7, TBP, NAMPT, IL18BP;
(vi) CXCL11, CD74, IL32, SERPINA1, B2M, TBP, NAMPT, IFNGR1, IL18BP; or
(vii) CXCL11, CD74, IL32, SERPINA1, B2M, TBP, NAMPT, IRAK2, IL18BP
in microvesicular RNA isolated from a biological sample from the subject;

b) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 37. A method of identifying kidney transplant rejection in a subject who has undergone a kidney transplant, the method comprising:

a) determining the expression level of:
(i) CXCL11, CD74, IL32, STAT1, SERPINA1, B2M, TBP, NAMPT, IL18BP;
(ii) CXCL11, CD74, IL32, CXCL14, SERPINA1, B2M, TBP, NAMPT. IL1BP;
(iii) CXCL11, CD74, IL32, SERPINA1, B2M, C3, TBP, NAMPT. 11,18BP;
(iv) CXCL11, CD74, IL32, SERPINA1, B211/1, PYCARD, TBP, NAMPT, IL18BP;
(v) CXCL11, CD74, IL32, SERPINA1, B2M, BMP7, TBP, NAMPT, IL18BP;
(vi) CXCL11, CD74, IL32, SERPINA1, B2M, TBP, NAMPT, IFNGR1, IL18BP; or
(vii) CXCL11, CD74, IL32, SERPINA1, B2M, TBP, NAMPT, IRAK2, IL18BP
in microvesicular RNA and cell-free DNA (cfDNA) isolated from a biological sample from the subject;

h) inputting the expression levels from step (a) into an algorithm to generate a score;

c) comparing the score to a predetermined cutoff value;

d) identifying kidney transplant rejection in the subject when the score is greater than or equal to the predetermined cutoff value or identifying the lack of kidney transplant rejection in the subject when the score is less than the predetermined cutoff value.

Embodiment 38. The method of any one of the preceding embodiments, wherein step (a) comprises determining the expression level of CXCL11, CD74, IL32, STAT1, SERPINA1, B2M, TBP, NAMPT, IL18BP.

Embodiment 39. The method of any one of the preceding embodiments, wherein step (a) comprises determining the expression level of CXCL11, CD74, IL32, CXCL14, SERPINA1, B2M, TBP, NAMPT, IL18BP.

Embodiment 40. The method of any one of the preceding embodiments, wherein step (a) comprises determining the expression level of CXCL11, CD74, IL32, SERPINA1, B2M, C3, TBP, NAMPT, IL18BP.

Embodiment 41. The method of any one of the preceding embodiments, wherein step (a) comprises determining the expression level of CXCL11, CD74, IL32, SERPINA1, B2M, PYCARD, TBP, NAMPT, IL18BP.

Embodiment 42. The method of any one of the preceding embodiments, wherein step (a) comprises determining the expression level of CXCL11, CD74, IL32, SERPINA1, B2M, BMP7, TBP, NAMPT, IL18BP

Embodiment 43. The method of any one of the preceding embodiments, wherein step (a) comprises determining the expression level of CXCL11, CD74, IL32, SERPINA1, B2M, TBP, NAMPT, IL18BP.

Embodiment 44. The method of any one of the preceding embodiments, wherein step (a) comprises determining the expression level of CXCL11, CD74, IL32, SERPINA1, B2M, TBP, NAMPT, IRAK2, IL18BP.

Embodiment 45. A method of identifying cell-mediated kidney transplant rejection in a subject who has undergone a kidney transplant, the method comprising:

a) determining the expression level of at least two of 13 biomarkers in microvesicular RNA isolated from a biological sample from the subject, wherein the 13 biomarkers comprise CD74, CXCL11, C3, CCL2, B2M, IL15, IL18BP, FPR2, ALOX5AP, IL1RAP, TLR1, NAMPT and IL1R2;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 46. A method of identifying cell-mediated kidney transplant rejection in a subject who has undergone a kidney transplant, the method comprising:

a) determining the expression level of at least two of 13 biomarkers microvesicular RNA and cell-free DNA (cfDNA) isolated from a biological sample from the subject, wherein the 13 biomarkers comprise CD74, CXCL 11, C3, CCL2, B2M, IL15, IL18BP, FPR2, ALOX5AP, IL1RAP, TLR1, NAMPT and IL1R2;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 47. The method of any of the preceding embodiments, wherein step (a) comprises determining the expression level of at least three of the 13 biomarkers.

Embodiment 48. The method of any of the preceding embodiments, wherein step (a) comprises determining the expression level of at least four of the 13 biomarkers.

Embodiment 49. The method of any of the preceding embodiments, wherein step (a) comprises determining the expression level of at least five of the 13 biomarkers.

Embodiment 50, The method of any of the preceding embodiments, wherein step (a) comprises determining the expression level of at least six of the 13 biomarkers,

Embodiment 51. The method of any of the preceding embodiments, wherein step (a) comprises determining the expression level of at least seven of the 13 biomarkers,

Embodiment 52. The method of any of the preceding embodiments, wherein step (a) comprises determining the expression level of at least eight of the 13 biomarkers.

Embodiment 53. The method of any of the preceding embodiments, wherein step (a) comprises determining the expression level of at least nine of the 13 biomarkers.

Embodiment 54. The method of any of the preceding embodiments, wherein step (a) comprises determining the expression level of at least ten of the 13 biomarkers.

Embodiment 55. The method of any of the preceding embodiments, wherein step (a.) comprises determining the expression level of at least 11 of the 13 biomarkers.

Embodiment 56. The method of any of the preceding embodiments, wherein step (a) comprises determining the expression level of at least 12 of the 13 biomarkers.

Embodiment 57. The method of any of the preceding embodiments, wherein step (a) comprises determining the expression level of each of the 13 biomarkers.

Embodiment 58. A method of identifying cell-mediated kidney transplant rejection in a subject who has undergone a kidney transplant, the method comprising:

a) determining the expression level of:
(i) CD74, CXCL11, C3, CCL2, B2M, IL15, IL18BP and FPR2;
(ii) CD74, CXCL11, C3, CCL2, B2M, IL15 and IL18BP);
(iii) CD74, CXCL11, C3, CCL2. B2M and IL15;
(iv) CD74, CXCL11, C3, CCL2 and B2M;
(v) CD74, CXCL11, C3 and CCL2;
(vi) CD74, CXCL11 and C3;
(vii) CD74 and CXCL11;
in microvesicular RNA isolated from a biological sample from the subject;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 59. A method of identifying cell-mediated kidney transplant rejection in a subject who has undergone a kidney transplant, the method comprising:

a) determining the expression level of
(i) CD74, CXCL11, C3, CCL2, B2M, IL15, IL18BP and FPR2;
(ii) CD74, CXCL11, C3, CCL2, B2M, IL15 and IL18BP;
(iii) CD74, CXCL11, C3, CCL2, B2M and IL15;
(iv) CD74, CXCL11, C3, CCL2 and B2M;
(v) CD74, CXCL11, C3 and CCL2;
(vi) CD74, CXCL11 and C3;
(vii) CD74 and CXCL11;
in microvesicular RNA and cell-free DNA (ctDNA) isolated from a biological sample from the subject;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 60. The method of any one of the preceding embodiments, wherein step (a) comprises determining the expression level of CD74, CXCL11, C3, CCL2, B2M, IL15, IL18BP and FPR2.

Embodiment 61. The method of any one of the preceding embodiments, wherein step (a) comprises determining the expression level of CD74, CXCL11, C3, CCL2, B2M, IL15 and IL18BP.

Embodiment 62. The method of any one of the preceding embodiments, wherein step (a) comprises determining the expression level of CD74, CDCL11, CS, CCL2, B2M and IL15.

Embodiment 63. The method of any one of the preceding embodiments, wherein step (a) comprises determining the expression level of CD74, CXCL11, C3, CCL2 and B2M.

Embodiment 64. The method of any one of the preceding embodiments, wherein step (a) comprises determining the expression level of CD74, CXCL11, C3 and CCL2.

Embodiment 65. The method of any one of the preceding embodiments, wherein step (a) comprises determining the expression level of CD74, CXCL11 and C3.

Embodiment 66. The method of any one of the preceding embodiments, wherein step (a) comprises determining the expression level of CD74 and CXCL11,

Embodiment 67. A method of identifying cell-mediated kidney transplant rejection in a subject who has undergone a kidney transplant, the method comprising:

a) determining the expression level of:
(i) CD74. CXCL11, C3, IL1RAP;
(ii) CD74, C3, IL1RAP;
(iii) CXCL11, C3, IL1RAP;
(iv) CD74, CXCL11, C3, CCL2, IL1RAP;
(v) CD74, CXCL11, C3, B2M, IL1RAP;
(vi) CD74, CDCL11, C3, IL15, IL1RAP,
(vii) CD74, CXCL11, C3, IL18BP, IL1RAP;
(viii) CD74, CXCL11, C3, FPR2, IL1RAP; or
(ix) CD74, CXCL11, C3, ALOX5AP, IL1RAP;
in microvesicular RNA isolated from a biological sample from the subject;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 68. A method of identifying cell-mediated kidney transplant rejection in a subject who has undergone a kidney transplant, the method comprising:

a) determining the expression level of:
(i) CD74, CXCL11, CS, IL1RAP;
(ii) CD74, C3, IL1RAP;
(iii) CXCL11, C3, IL1RAP;
(iv) CD74, CXCL11, C3, CCL2, IL1RAP;
(v) CD74, CXCL11, C3, B2M, IL1RAP;
(vi) CD74, CXCL11, C3, IL15, IL1RAP;
(vii) CD74, CXCL11, C3, IL18BP, IL1RAP,
(viii) CD74. CXCL11, C3, FPR2, IL1RAP; or
(ix) CD74, CXCL11, C3, ALOX5AP, IL1RAP;
in microvesicular RNA and cell-free DNA (ctDNA) isolated from a biological sample from the subject;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 69. The method of any one of the preceding embodiments, wherein step (a) comprises determining the expression level of CD74, CXCL11, CS, IL1RAP.

Embodiment 70. The method of any one of the preceding embodiments, wherein step (a) comprises determining the expression level of CD74, C3, IL1RAP.

Embodiment 71. The method of any one of the preceding embodiments, wherein step (a) comprises determining the expression level of CXCL11, C3, IL1RAP.

Embodiment 72. The method of any one of the preceding embodiments, wherein step (a) comprises determining the expression level of CD74, CXCL11, C3, CCL2, IL1RAP.

Embodiment 73. The method of any one of the preceding embodiments, wherein step (a) comprises determining the expression level of CD74. CXCL11, C3, B2M, IL1RAP.

Embodiment 74. The method of any one of the preceding embodiments, wherein step (a) comprises determining the expression level of CD74, CXCL11, C3, IL15, IL1RAP.

Embodiment 75. The method of any one of the preceding embodiments, wherein step (a) comprises determining the expression level of CD74, CXCL11, C3, IL18BP , IL1RAP.

Embodiment 76. The method of any one of the preceding embodiments, wherein step (a) comprises determining the expression level of CD74, CXCL11, C3, FPR2,

Embodiment 77. The method of any one of the preceding embodiments, wherein step (a) comprises determining the expression level of CD74, CXCL11, C3, ALOX5AP, IL1RAP.

Embodiment 78. A method of identifying cell-mediated kidney transplant rejection in a subject who has undergone a kidney transplant, the method comprising:

a) determining the expression level of:
(i) CD74, CXCL11, C3, CCL2, B2M, IL15, IL18BP, FPR2, ALOX5AP, TLR1, NAMPT, IL1R2 ; or
(ii) CD74, CXCL11, CCL2, B2M, IL15, IL18BP, FPR2, ALOX5AP, IL1RAP, TLR1, NAMPT, IL1R2
microvesicular RNA isolated from a biological sample from the subject;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 79. A method of identifying cell-mediated kidney transplant rejection in a subject who has undergone a kidney transplant, the method comprising:

a) determining the expression level of:
(i) CD74, CXCL11, C3, CCL2, B2M, IL15, IL18BP, FPR2, ALOX5AP, TLR1, NAMPT, I R2 ; or
(ii) CD74, CXCL11, CCL2, B2M, IL15, IL18BP, FPR2, ALOX5AP, IL1RAP, TLR1, NAMPT, IL1R2
in microvesicular RNA and cell-free DNA (cfDNA) isolated from a biological sample from the subject;

b) inputting the expression levels from step (a) into an algorithm to generate a score;

c) comparing the score to a predetermined cutoff value;

d) identifying cell-mediated kidney transplant rejection in the subject when the score is greater than or equal to the predetermined cutoff value or identifying the lack of cell-mediated kidney transplant rejection in the subject when the score is less than the predetermined cutoff value.

Embodiment 80. The method of any one of the preceding embodiments, wherein step (a) comprises determining the expression level of CD74, CXCL11, C3, CCL2, B2M, IL15, IL18BP, FPR2, ALOX5AP, TLR1, NAMPT, IL1R2.

Embodiment 81. The method of any one of the preceding embodiments, wherein step (a) comprises determining the expression level of CD74, CXCL11, CCL2, B2M, IL15, IL18BP, FPR2, ALOX5AP, IL1RAP, TLR1, NAMPT, IL1R2,

Embodiment 82. A method of identifying antibody-mediated kidney transplant rejection in a subject who has undergone a kidney transplant, the method comprising:

a) determining the expression level of al least two of 13 biomarkers in microvesicular RNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 83. A method of identifying antibody-mediated kidney transplant rejection in a subject who has undergone a kidney transplant, the method comprising:

a) determining the expression level of at least two of 13 biomarkers in microvesicular RNA and cell-free DNA (cfDNA) isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 84, The method of any of the preceding embodiments, wherein step (a) comprises determining the expression level of at least three of the 13 biomarkers.

Embodiment 85. The method of any of the preceding embodiments, wherein step (a) comprises determining the expression level of at least four of the 13 biomarkers.

Embodiment 86. The method of any of the preceding embodiments, wherein step (a) comprises determining the expression level of at least five of the 13 biomarkers.

Embodiment 87. The method of any of the preceding embodiments, wherein step (a) comprises determining the expression level of at least six of the 13 biomarkers.

Embodiment 88. The method of any of the preceding embodiments, wherein step (a) comprises determining the expression level of at least seven of the 13 biomarkers.

Embodiment 89, The method of any of the preceding embodiments, wherein step (a) comprises determining the expression level of at least eight of the 13 biomarkers,

Embodiment 90. The method of any of the preceding embodiments, wherein step (a) comprises determining the expression level of at least nine of the 13 biomarkers.

Embodiment 91. The method of any of the preceding embodiments, wherein step (a) comprises determining the expression level of at least ten of the 13 biomarkers.

Embodiment 92. The method of any of the preceding embodiments, wherein step (a) comprises determining the expression level of at least 11 of the 13 biomarkers.

Embodiment 93, The method of any of the preceding embodiments, wherein step (a) comprises determining the expression level of at least 12 of the 13 biomarkers,

Embodiment. 94. The method of any of the preceding embodiments, wherein step (a) comprises determining the expression level of each of the 13 biomarkers.

Embodiment 95. A method of identifying antibody-mediated kidney transplant rejection in a subject who has undergone a kidney transplant, the method comprising:

a) determining the expression level of:
(i) CD44, NAMPT, PYCARD, IRAK2, IL32, TBP, BCL10, IFNGR1, BMP7 and STAT1;
(ii) CD44, NAMPT, PYCARD, IRAK2, 1L32, TBP, BCL10, IFNGR1 and BMP7;
(iii) CD44, NAMPT, PYCARD, IRAK2, IL32, TBP, BCL10 and IFNGR1;
(iv) CD44, NAMPT, PYCARD, IRAK2, IL32, TBP and BCL10;
(v) CD44, NAMPT, PYCARD, IRAK2, IL32 and TBP;
(vi) CD44, NAMPT, PYCARD, 1RAK2 and IL32;
(vii) CD44, NAMPT, PYCARD and IRAK2;
(viii) CD44, NAMPT and PYCARD; or
(ix) CD44 and NAMPT
in microvesicular RNA isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 96. A method of identifying antibody-mediated kidney transplant rejection in a. subject who has undergone a kidney transplant, the method comprising:

a) determining the expression level of
(i) CD44, NAMPT, PYCARD, IL32, TBP, BCL10, IFNGR1, BMP7 and STAT1;
(ii) CD44, NAMPT, PYCARF IR K2, IL32, TBP, BCH 0, IFNGR1 d BMP7;
(iii) CD44, NAMPT, PYCARD, IRAK2, IL32, TBP, BCL10 and IFINGR1;
(iv) CD44, NAMPT, PYCARD, IRAK2, IL32, TBP and BCL10;
(v) CD44, NAMPT, PYCARD, IRAK2, IL32 and TBP;
(vi) CD44, NAMPT, PYCARD, IRAK2 and IL32,
(vii) CD44, NAMPT, PYCARD and IRAK2;
(viii) CD44, NAMPT and PYCARD; or
(ix) CD44 and NAMPT
in microvesicular RNA and cell-free DNA (cfDNA) isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 97. The method of any one of the preceding embodiments, wherein step (a) comprises determining the expression level of CD44, NAMPT, PYCARD, IRAK2, IL32, TBP, BCL10, IFNGR1, BMP7 and STAT1.

Embodiment 98. The method of any one of the preceding embodiments, wherein step (a) comprises determining the expression level of CD44, NAMPT PYCARD, IRAK2, IL32, TBP, BCL10, IFNGR1 and BMP7.

Embodiment 99. The method of any one of the preceding embodiments, wherein step (a) comprises determining the expression level of CD44, NAMPT, PYCARD, IRAK2, IL32, TBP, BCL10 and IFNGR1.

Embodiment 100. The method of any one of the preceding embodiments, wherein step (a) comprises determining the expression level of CD44, NAMPT, PYCARD, IRAK2, IL32, TBP and BCL10.

Embodiment 101. The method of any one of the preceding embodiments, wherein step (a) comprises determining the expression level of CD44, NAMPT, PYCARD, IRAK2, IL32 and TBP.

Embodiment 102, The method of any one of the preceding embodiments, wherein step (a) comprises determining the expression level of CD44, NAMPT, PYCARD, IRAK2 and

Embodiment 103. The method of any one of the preceding embodiments, wherein step (a) comprises determining the expression level of CD44, NAMPT, PYCARD and IRAK2.

Embodiment 104. The method of any one of the preceding embodiments, wherein step (a) comprises determining the expression level of CD44, NAMPT and PYCARD.

Embodiment 105, The method of any one of the preceding embodiments, wherein step (a) comprises determining the expression level of CD44 and NAMPT.

Embodiment 106. A method of identifying antibody-mediated kidney transplant rejection in a subject who has undergone a kidney transplant, the method comprising:

a) determining the expression level of:
(i) CD44, PYCARD, IRAK2, IL32, IFNGR1, BMP7, STAT1;
(ii) NAMPT, PYCARD, IRAK2, IL32, IFNGR1, BMP7, STAT1;
(iii) PYCARD, IRAK2, IL32, TBP, IFNGR1, BMP7, STAT1;
(iv) PYCARD, IRAK2, IL32, BCL10, IFNGR1, BMP7, STAT1;
(v) CD44, NAMPT, PYCARD, IRAK2, IL32, IFNGR1, BMP7, STAT1;
(vi) CD44, NAMPT, PYCARD, IRAK2, IL32, BCL10, IFNGR1, BMP7, STAT1; or
(vii) CD44, NAMPT, PYCARD, IRAK2, IL32, TBP, IFNGR1, BMP7, STAT1
in microvesicular RNA isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 107. A method of identifying antibody-mediated kidney transplant rejection in a subject who has undergone a kidney transplant, the method comprising:

a) determining the expression level of:
(i) CD44, PYCARD, TRAK2, IL32, IFNGR1, BMP7, STAT1;
(ii) NAMPT, PYCARD, IRAK2, 1L32, IFNGR1, BMP7, STAT1;
(iii) PYCARD, IRAK2, IL32, TBP, IFNGR1, BMP7, STAT1;
(iv) PYCARD, IRAK2, IL32, BCL10, IFNGR1, BMP7, STAT1;
(v) CD44, NAMPT, PYCARD, IRAK2, IL32, IFNGR1, BMP7, STAT1;
(vi) CD44, NAMPT, PYCARD, IRAK2, 1L32, BCL10, IFNGR1, BMP7, STAT1; or
(vii) CD44, NAMPT, PYCARD, IRAK2, IL32, TBP IFNGR1, BMP7, STAT 1
in microvesicular RNA and cell-free DNA (cfDNA) isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 108. The method of any one of the preceding embodiments, wherein step (a) comprises determining the expression level of CD44, PYCARD, IRAK2, IL32, IFNGR1, BMP7, STAT1.

Embodiment 109. The method of any one of the preceding embodiments, wherein step (a) comprises determining the expression level of NAMPT, PYCARD, IRAK2, IL32, IFNGR1, BMP7, STAT1.

Embodiment 110. The method of any one of the preceding embodiments, wherein step (a) comprises determining the expression level of .PYCARD, IRAK2, IL32, TBP, BMP7, STAT1.

Embodiment 111. The method of any one of the preceding embodiments, wherein step (a) comprises determining the expression level of PYCARD, IRAK2, IL32, BCL10, IFNGR1, IBMP7, STAT1.

Embodiment 112. The method of any one of the preceding embodiments, wherein step (a) comprises determining the expression level of CD44, NAMPT, PYCARD, IRAK2, IL32, IFNGR1 BMP7, STAT1,

Embodiment 113. The method of any one of the preceding embodiments, wherein step (a) comprises determining the expression level of CD44, NAMPT, PYCARD, IRAK2, IL32, BCL10, IFNGR1, BMP7, STAT1

Embodiment 114. The method of any one of the preceding embodiments, wherein step (a) comprises determining the expression level of CD44, NAMPT, PYCARD, IRAK2, IL32, TBP, IFNGR1, BMP7, STAT 1 .

Embodiment 115, A method of identifying antibody-mediated kidney transplant rejection in a subject who has undergone a kidney transplant, the method comprising:

a) determining the expression level of:
(i) CD44, NAMPT, PYCARD, TBP, BCL10, ANXA1, TYMP, NFX1;
(ii) CD44, NAMPT, IRAK2, TBP, ANXA1, TYMP, NFX1;
(iii) CD44, NAMPT, IL32, TBP, BCL10, ANXA1, TYMP, NFX1;
(iv) CD44, NAMPT, TBP. BCL10, IFNGR1, ANXA1, TYMP, NFX1;
(v) CD44, NAMPT, TBP, BCL10, BMP7, ANXA1, TYMP, NFX1; or
(vi) CD44, NAMPT, TBP, BCL10, STAT1, ANXA1, TYMP, NFX1
in microvesicular RNA isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 116. A method of identifying antibody-mediated kidney transplant rejection in a subject who has undergone a kidney transplant, the method comprising:

a) determining the expression level of:
(i) CD44, NAMPT, PYCARD, TBP, BCL10, ANXA1, TYMP, NFX1;
(ii) CD44, NAMPT, IRAK2, TBP, BCL10, ANXA1, TYMP, NFX1;
(iii) CD44, NAMPT, IL32, TBP, BCL10, ANXA1, TYMP, NFX1;
(iv) CD44, NAMPT, TBP, BCL10, IFNGR1, ANXA1, TYMP, NFX1;
(v) CD44, NAMPT, TBP, BCL10, BMP7, ANXA1, TYMP, NFX1; or
(vi) CD44, NAMPT, TBP, BCL10, STAT1, ANXA1, TYMP, NFX1
in micro vesicular RNA and cell-free DNA (cfDNA) isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the lack of antibody-mediated kidney transplant rejection in the subject when the score is less than the predetermined cutoff value.

Embodiment 117, The method of any one of the preceding embodiments, wherein step (a) comprises determining the expression level of CD44, NAMPT, PYCARD, TBP, BCL10, ANXA1, TYMP, NFX1.

Embodiment 118. The method of any one of the preceding embodiments, wherein step (a) comprises determining the expression level of CD44, NAMPT, IRAK2, TBP, BCL10, ANXA1, TYMP, NFX1.

Embodiment 119. The method of any one of the preceding embodiments, wherein step (a) comprises determining the expression level of CD44, NAMPT, IL32, TBP, BCL10, ANXA1, TYMP, NFX1.

Embodiment 120. The method of any one of the preceding embodiments, wherein step (a) comprises determining the expression level of CD44, NAMPT, TBP, BCL10, IFNGR1, ANXA1, TYMP, NFX1.

Embodiment 121. The method of any one of the preceding embodiments, wherein step (a) comprises determining the expression level of CD44, NAMPT, TBP, BCL10, BMP7, ANXA1, TYMP, NFX1.

Embodiment 122, The method of any one of the preceding embodiments, wherein step (a) comprises determining the expression level of CD44, NAMPT, TBP, BCL10, STAT1, ANXA1, TYMP, NFX1.

Embodiment 123. A method of identifying antibody-mediated kidney transplant rejection or cell-mediated kidney transplant rejection in a subject who has undergone a kidney transplant and has been identified as having a kidney transplant rejection, the method comprising:

a) determining the expression level of at least two of five biomarkers in microvesicular RNA isolated from a biological sample from the subject, wherein the five biomarkers comprise CD74, C3, CXCL11, CD44 and IFNAR2;

b) inputting the expression levels from step (a) into an algorithm to generate a score;

c) comparing the score to a predetermined cutoff value;

d) antibody-mediated. kidney transplant rejection in the subject when the score is greater than or equal to the predetermined cutoff value or identifying the cell-mediated kidney transplant rejection in the subject when the score is less than the predetermined cutoff value.

Embodiment 124. A method of identifying antibody-mediated kidney transplant rejection or cell-mediated kidney transplant rejection in a subject who has undergone a kidney transplant and has been identified as having a kidney transplant rejection, the method comprising:

a) determining the expression level of at least two of five biomarkers in microvesicular RNA and cell-free DNA (cfDNA) isolated from a biological sample from the subject, wherein the five biomarkers comprise CD74, C3, CXCL11, CD44 and 1FNAR2;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the cell-mediated kidney transplant rejection in the subject when the score is less than the predetermined cutoff value.

Embodiment 125. The method of any of the preceding embodiments, wherein step (a) comprises determining the expression level of at least three of the 5 biomarkers.

Embodiment 126. The method of any of the preceding embodiments, wherein step (a) comprises determining the expression level of at least four of the 5 biomarkers.

Embodiment 127, The method of any of the preceding embodiments, wherein step (a) comprises determining the expression level of each of the 5 biomarkers.

Embodiment 128. The method of any one of the preceding embodiments, wherein the subject has been identified as having a kidney transplant rejection using the method of any one of preceding embodiments.

Embodiment 129. A method of identifying antibody-mediated kidney transplant rejection or cell-mediated kidney transplant rejection in a subject who has undergone a kidney transplant and has been identified as having a kidney transplant rejection, the method comprising:

a) determining the expression level of:
(i) CD74, C3, CXCL11 and CD44;
(ii) CD74, C3 and CXCL11; or
(iii) CD74 and C3
in microvesicular RNA isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the cell-mediated kidney transplant rejection in the subject when the score is less than the predetermined cutoff value.

Embodiment 130. A method of identifying antibody-mediated kidney transplant rejection or cell-mediated kidney transplant rejection in a subject who has undergone a kidney transplant and has been identified as having a kidney transplant rejection, the method comprising:

a) determining the expression level of
(i) CD74, C3, CXCL11 and CD44;
(ii) CD74, C3 and CXCL11; or
(iii) CD74 and C3
in microvesicular RNA and cell-free DNA (cfDNA) isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the cell-mediated kidney transplant rejection in the subject when the score is less than the predetermined cutoff value.

Embodiment 131. The method of any one of the preceding embodiments, wherein step (a) comprises determining the expression level of CD74, C3, CXCL11 and CD44.

Embodiment 132. The method of any one of the preceding embodiments, wherein step (a) comprises determining the expression level of CD74. C3 and CXCL11.

Embodiment 133. The method of any one of the preceding embodiments, wherein step (a) comprises determining the expression level of CD74 and C3.

Embodiment 134. A method of identifying antibody-mediated kidney transplant rejection or cell-mediated kidney transplant rejection in a subject who has undergone a kidney transplant and has been identified as having a kidney transplant rejection, the method comprising:

a) determining the expression level of
(i) C3, CXCL11;
(ii) C3, CD44;
(iii) C3, CXCL11, CD44; or
(iv) CD74, C3, CD44
in microvesicular RNA isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the cell-mediated kidney transplant rejection in the subject when the score is less than the predetermined cutoff value.

Embodiment 135. A method of identifying antibody-mediated kidney transplant rejection or cell-mediated kidney transplant rejection in a subject who has undergone a kidney transplant and has been identified as having a kidney transplant rejection, the method comprising:

a) determining the expression level of
(i) C3, CXCL11;
(ii) C3, CD44;
(iii) C3, CXCL11, CD44; or
(iv) CD74, C3, CD44
in microvesicular RNA and cell-free DNA (ctDNA) isolated from a biological sample from the subject;

b) inputting the expression levels from step (a) into an algorithm to generate a score;

c) comparing the score to a predetermined cutoff value;

d) identifying antibody-mediated kidney transplant rejection in the subject when the score is greater than or equal to the predetermined cutoff value or identifying the cell-mediated kidney transplant rejection in the subject when the score is less than the predetermined cutoff value.

Embodiment 136, The method of any one of the preceding embodiments, wherein step (a) comprises determining the expression level of C3, CXCL11.

Embodiment 137. The method of any one of the preceding embodiments, wherein step (a) comprises determining the expression level of C3, CD44.

Embodiment 138. The method of any one of the preceding embodiments, wherein step (a) comprises determining the expression level of C3, CXCL11, CD44.

Embodiment 139, The method of any one of the preceding embodiments, wherein step (a) comprises determining the expression level of CD74, C3, CD44.

Embodiment 140. The method of any of the preceding embodiments, further comprising prior to step (a):

i) isolating a plurality of microvesicles from a biological sample from the subject; and

ii) extracting at least one microvesicular RNA from the plurality of isolated microvesicles.

Embodiment 141. The method of any of the preceding embodiments, further comprising prior to step (a):

i) isolating a microvesicle fraction from a biological sample from the subject, wherein the microvesicle fraction comprises a plurality of microvesicles and cfDNA; and

ii) extracting at least one microvesicular RNA and at least one cfDNA molecule from the plurality of isolated microvesicles.

Embodiment 142. The method of any of the preceding embodiments, wherein isolating a plurality of microvesicles from a biological sample from the subject comprises a processing step to remove cells, cellular debris or a combination of cells and cellular debris.

Embodiment 143. The method of any of the preceding embodiments, wherein the processing step comprises filtering the sample, centrifuging the sample, or a combination of filtering the sample and centrifuging the sample.

Embodiment 144. The method of any of the preceding embodiments, wherein centrifuging comprises centrifuging at about 2000×g.

Embodiment 14.5, The method of any of the preceding embodiments, wherein filtering comprises filtering the sample through a filter with a pore size of about 0.8 microns.

Embodiment 146. The method of any of the preceding embodiments, wherein isolating a plurality of microvesicles comprises ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

Embodiment 147. The method of any of the preceding embodiments, wherein the at least one microvesicle is isolated from the bodily fluid sample by contacting the bodily fluid sample with at least one affinity agent that binds to at least one surface marker present on the surface the at least one microvesicle.

Embodiment 148, The method of any of the preceding embodiments, wherein the biological sample is a urine sample.

Embodiment 149. The method of any of the preceding embodiments, wherein the biological sample is a first-catch urine sample.

Embodiment 150. The method of any of the preceding embodiments, wherein the biological sample is a second voided urine sample.

Embodiment 151. The method of any of the preceding embodiments, wherein the biological sample has a volume of between at least about 1 ml to at least about 50 ml.

Embodiment 152. The method of any of the preceding embodiments, wherein the biological sample has a volume of up to about 20 ml.

Embodiment 153. The method of any of the preceding embodiments, wherein step (a) further comprises:

(i) determining the expression level of at least one reference biomarker;

(ii) normalizing the expression level of the at least two, or the at least three, or the at least four, of the at least five, or at the at least six, or the at least seven, or the at least eight, or the at least nine, or the at least 10, or the at least 11, or the at least 12. or the at least 13 biomarkers to the expression level of the at least one reference biomarker.

Embodiment 154. The method of any of the preceding embodiments, wherein inputting the expression levels from step (a) into an algorithm to generate a score comprises inputting the normalized expression levels from step (a) into an algorithm to generate a score.

Embodiment 155. The method of any of the preceding embodiments, wherein the at least one reference biomarker comprises PGK1.

Embodiment 156. The method of any of the preceding embodiments, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis or any combination thereof

Embodiment 157. The method of any of the preceding embodiments, wherein the predetermined cutoff value has a negative predictive value of at least about 80%, or at least about 85%, or at least about 90%, or at least about 95%, or at least about 99%, or at least about

Embodiment 158. The method of any of the preceding embodiments, wherein the predetermined cutoff value has a positive predictive value of at least about 80%, or at least about 85%, or at least about 90%, or at least about 95%, or at least about 99%, or at least about 99.9%.

Embodiment 159. The method of any of the preceding embodiments, wherein the predetermined cutoff value has a sensitivity of at least about 80%, or at least about 85%, or at least about 90%, or at least about 95%, or at least about 99%, or at least about 99.9%.

Embodiment 160. The method of any of the preceding embodiments, wherein the predetermined cutoff value has a. specificity of at least about 80%, or at least about 85%, or at least about 90%, or at least about 95%, or at least about 99%, or at least about 99.9%.

Embodiment 161. The method of any of the preceding embodiments, wherein the kidney transplant rejection is an any-cause kidney transplant rejection.

Embodiment 162. The method of any of the preceding embodiments, wherein the algorithm is the product of a feature selection wrapper algorithm,

Embodiment 163. The method of any of the preceding embodiments, wherein the algorithm is the product of a machine learning algorithm.

Embodiment 164. The method of any of the preceding embodiments, wherein the algorithm is the product of a trained classifier built from at least one predictive classification algorithm.

Embodiment 165. The method of any of the preceding embodiments, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), cforest, classification and regression tree (CART), treebag, k nearest-neighbor (knn), neural network (nnet), support, vector machine-radial (SVM-radial), support vector machine-linear (SVM-linear), naïve bayes (NB), multilayer perceptron (mlp) or any combination thereof.

Embodiment 166. The method of any of the preceding embodiments, wherein the algorithm is the product of a logistic regression model.

Embodiment 167. The method of any of the preceding embodiments, wherein the logistic regression model comprises a LASSO regularization.

Embodiment 168, The method of any of the preceding embodiments, wherein the predetermined cutoff value is calculated using at least one receiver operating characteristic (ROC) curve.

Embodiment 169. The method of any of the preceding embodiments, wherein the algorithm is a product of a feature selection wrapper algorithm, machine learning algorithm, trained classifier, logistic regression model or any combination thereof, that was trained to identify kidney transplant rejection in a subject using:

a) the expression levels of the at least two, or the at least three, or the at least four, or the at least five, or the at least six, or the at least seven, or the at least eight, or the at least nine, or the at least 10, or the at least 11, or the at least 12, or the at least 13 biomarkers, or the at least 14 biomarkers, or the at least 15 biomarkers in at least one biological sample from at least one subject who is kidney transplant rejection negative; and

b) the expression levels of the at least two, or the at least three, or the at least four, or the at least five, or the at least six, or the at least seven, or the at least eight, or the at least nine, or the at least 10, or the at least 11, or the at least 12, or the at least 13 biomarkers, or the at least 14 biomarkers, or the at least 15 biomarkers in at least one biological sample from at least one subject who is kidney transplant rejection positive.

Embodiment 170. The method of any of the preceding embodiments, wherein the at least one subject who is kidney transplant rejection negative is determined to be kidney transplant rejection negative based on kidney transplant biopsy results.

Embodiment 171. The method of any of the preceding embodiments, wherein the at least one subject who is kidney transplant rejection positive is determined to be kidney transplant rejection positive based on kidney transplant biopsy results.

Embodiment 172. The method of any of the preceding embodiments, further comprising administering at least one kidney transplant rejection therapy to a subject identified as having kidney transplant rejection.

Embodiment 173. The method of any of the preceding embodiments, wherein the at least one kidney transplant rejection therapy comprises administering to the subject at least one therapeutically effective amount of at least one immunosuppressant, at least one therapeutically effective amount of at least one corticosteroid, at least one therapeutically effective amount of at least one steroid, at least one therapeutically effective amount of at least one anti-T-cell antibody, at least one therapeutically effective amount of mycophenolate mofetil (MMF), at least one therapeutically effective amount of cyclosporine A (CsA), at least one therapeutically effective amount of tacrolimus, at least one therapeutically effective amount of azathioprine, at least one therapeutically effective amount of muromonab (OKT-3), at least one therapeutically effective amount of anti-thymocyte globulin (ATG), at least one therapeutically effective amount of anti-lymphocyte globulin (ALG) or any combination thereof.

Embodiment 174. The method of any one of the preceding embodiments, further comprising administering to a subject identified as being at risk for a kidney transplant rejection at least one kidney transplant rejection therapy.

Embodiment 175. The method of any one of the preceding embodiments, wherein the at least one kidney transplant rejection therapy comprises administering to the subject at least one therapeutically effective amount of at least one immunosuppressant, at least one steroid, at least one corticosteroid, at least one anti-T-cell antibody, mycophenolate mofetil (MMF), cyclosporine A (CsA), tacrolimus, azathioprine, muromonab (OKT-3), anti-thymocyte globulin (ATG), anti-lymphocyte globulin (ALG), Campath (alemtuzumab), prednisone, mycophenolic acid, rapamycin, belatacept, intravenous immunoglobulin (IVIg), an anti-CD20 agent, rituxirnab, bortezomib, or any combination thereof.

Embodiment 176. The method of any one of the preceding embodiments, further comprising administering to a subject identified as being at risk for a cell-mediated kidney transplant rejection at least one cell-mediated kidney transplant rejection therapy.

Embodiment 177. The method of any one of the preceding embodiments, wherein the at least one cell-mediated kidney transplant rejection therapy comprises administering to the subject at least one therapeutically effective amount of at least one steroid, at least one corticosteroid, muromonab (OKT-3), anti-thymocyte globulin (ATG), Campath (alemtuzwnab), prednisone, tacrolimus cyclosporine A, mycophenolic acid, azathioprine, rapamycin, amount of belatacept, or any combination thereof.

Embodiment 178. The method of any one of the preceding embodiments, further comprising administering to a subject identified as being at risk for an antibody-mediated kidney transplant rejection at least one antibody-mediated kidney transplant rejection therapy.

Embodiment 179, The method of any one of the preceding embodiments, wherein the at least one antibody-mediated kidney transplant rejection therapy comprises administering to the subject at least one therapeutically effective amount of at least one steroid, at least one corticosteroid, anti-thymocyte globulin (ATG), intravenous immunoglobulin (IVIg), an anti-CD20 agent, rituximab, bortezomib, or any combination thereof.

Embodiment 180. The method of any one of the preceding embodiments, wherein the subject has not undergone a renal biopsy.

### EXAMPLES

### Example 1

The following example describes a study of 198 urine samples collected from 183 renal transplant patients used to derive the gene signatures for the detection of kidney rejection described herein. Of these 198 samples, 133 were kidney transplant rejection negative, 41 were cell-mediated kidney rejection positive and 24 were antibody mediated kidney transplant rejection positive.

Methods

Patient and Sample Information

After kidney transplantation, urine samples were collected from patients undergoing a transplant kidney biopsy for clinical indications. The training set included 26 of 28 urine samples from 23 patients (2 samples were rejected during RNA extraction). The validation set included 38 of 39 urine samples from 32 patients (1 sample was rejected during RNA extraction.), 13 urine samples in the training set and 23 urine samples in the validation set had signs of any-cause acute rejection after careful medical chart and allograft biopsy adjudication based on Banff Criteria, respectively.

Urinary Microvesicles Isolation, mRNA Extraction and Gene Expression Analysis

The urine samples used were second voided urine sample collected on the morning of the biopsy. The urine samples were stored at −80° C. Three in-house controls were also used, consisting of 1 pooled male sample, 1 pooled female sample, and 1 pooled male & female sample. Up to 20 ml urine were centrifuged to remove cells and cellular debris at 2000×g for 20 minutes. Exosomes were isolated from the urine supernatant using Exosome Diagnostics' EXOPRO Urine Clinical Sample Concentrator Kit as described previously in Meehan S M, Siegel C T, Aronson A J, Bartosh S M, Thistlethwaite J R, Woodle E S, et al. “The relationship of untreated borderline infiltrates by the Banff criteria to acute rejection in renal allograft biopsies.” J Am Soc Nephrol JASN. 1999 August; 10(8):1806-14, which is incorporated herein by reference in its entirety. RNA was eluted in 16 μl nuclease-free H₂O, 14 μl of which was used in a 20 μd. reverse transcription (RI) reaction using the VILO cDNA synthesis kit (Thermo Fisher).

The first round of samples was analyzed using the TaqMan® OpenArray® Human Inflammation Panel (Thermo Fisher). This panel consists of 586 TaqMan assays for genes that have been studied as targets for a range of inflammatory diseases and includes 21 endogenous control assays. To prepare the samples for quantitative PCR (qPCR), 10 μl cDNA was split into two, equal portions and pre-amplified with two pools of mixed primers following the manufacturer's directions. The pre-amplification reactions were mixed and diluted prior to mixing with TaqMan® OpenArray® Real-Time PCR Master Mix. Reaction mixes were loaded onto the OpenArray plates and the plates run on the QuantStudio™ 12K Flex real-time PCR system (Thermo Fisher) using the preset protocol for this panel.

After initial analysis, a subset of assays was identified and plated onto a custom TagMan OpenArray Panel This panel consisted of 112 TagMan assays. For this panel, 5 μl cDNA was pre-amplified with a pool of the 112 assays using the manufacturer's directions. The pre-amplification reactions were diluted prior to mixing samples TagMan® OpenArray® Real-Time PCR Master Mix. Reaction mixes were loaded onto the Open Array plates and the plates run on the QuantStudio™ 12K Flex real-time PCR system (Thermo Fisher) using the preset protocol for this panel.

Statistics

Genes with data missing from >30% of the samples was imputed using a non-parametric missing value imputation (missForest). The et values from OpenArray were normalized to PGK1. The Boruta package was used for feature selection. A logistic regression model with LASSO regularization was fit to the relevant features to generate the rejection probabilities. The pROC package was used to generate the ROC curves. The cut point was derived using the OptimalCutpoints package by setting the minimum NPV and PPV thresholds to 0.9 and 0.4 respectively.

Results

Patients' Characteristics and Biopsies

183 renal transplant patients who underwent a clinically indicated kidney transplant biopsy were enrolled in the present study. A total of 190 matched urine samples for biopsies were included to form the training and the validation cohorts. The biopsy-based pathologists' reports, based on Banff classification were used to discriminate any-cause rejection (including TCMR (Grades IA, IB, IIA, IIB, III), borderline rejection, active ABMR and chronic active ABMR) from no rejection status. Within the 112 urine samples from the training cohort and the 79 urine samples from the validation set, 38 and 27 were from patients with any-cause rejection based on Banff Criteria, respectively. Table 1 shows the baseline characteristics of the study cohorts.

TABLE 1

| | Training Cohort (n = 112) | | | | Testing Cohort (n = 78) | | | |
|---|---|---|---|---|---|---|---|---|
| | No | | Any-Cause | | No | | Any-Cause | |
| | Rejection | | Rejection | | Rejection | | Rejection | |
| Characteristic | (n = 75) | | (n = 37) | | (n = 52) | | (n = 26) | |
| Age, years? | 48.8 ± 21.9? | | 46.8 ± 12.4 | | 52.47 ± 15.4 | | 55.1 ± 17.6 | |
| Female, % | 29.3 | | 54.1 | | 34.6 | | 34.6 | |
| Race, % | | | | | | | | |
| White | 86.7 | | 78.4 | | 84.6 | | 76.9 | |
| Black | 13.3 | | 21.6 | | 15.4 | | 23.1 | |
| Time to Biopsy, | 1.7 | [0.4-2.7] | 38.1 | [3.1-116] | 2.0 | [0.9-37.8] | 21.3 | [3.9-50.3.] |
| months | | | | | | | | |
| SCr at Biopsy, mg/dl | 1.8 | [1.5-2.7] | 2.2 | [1.7-2.7] | 1.8 | [1.5-2.4] | 2.0 | [1.5-2.4] |
| Previous Transplant, | 21.3 | | 24.3 | | 21.2 | | 7.7 | |
| % | | | | | | | | |
| Previous Rejection, % | 16.0 | | 48.6 | | 9.6 | | 38.5 | |
| Induction Therapy | | | | | | | | |
| Thymoglobulin | 50.0 | | 57.1 | | 33.3 | | 47.8 | |
| Basiliximab | 50.0 | | 42.9 | | 46.7 | | 39.1 | |
| Deceased Donor, % | 46.7 | | 56.8 | | 42.3 | | 23.1 | |
| Cold Ischemia time, | 10 | [1.2-12] | 7.8 | [1.1-10.6] | 1.3 | [1.0-10.4] | 1.2 | [1.0-9.5] |
| hours | | | | | | | | |

| | | | | |
|---|---|---|---|---|
| Rejection Type, % | — | | — | |
| Cellular | | 62.2 | | 57.7 |
| Antibody Mediated | | 37.8 | | 42.3 |

The mean age of patients without rejection was 48.8 years in the training cohort and 52.4 years in the validation cohort. Similarly, patients with any-cause rejection were slightly younger in the training cohort 46.8±12,4 years vs 55.1±17.6). The active rejection subgroup included a higher proportion of black patients with previous rejection episodes as well as a longer time to biopsy in both training and validation cohorts. Proportion of deceased donor was higher in patients with any-cause rejection in the training cohort but not in the validation cohort. In the training cohort, 62.2% of rejection cases were due to acute TCMR, and 37.8% attributed to ABMR compared to 57.7% and 42.3% in the validation cohort, respectively. In a second time, we evaluated the performance of the median serum creatinine (SCr) level at biopsy in patients with any-cause rejection in the training cohort was 2.2 mg/dl and 2.0 mg/dl in the validation cohort.

Identifying an Any-Cause Kidney Transplant Rejection Gene Signature from Urinary Exosomes

mRNA from urinary exosomes (urinary microvesicular RNA) isolated from urine samples collected from patients with biopsy-confirmed any-cause rejection was compared to urinary microvesicular RNA from urine samples collected from patients identified as rejection-negative by biopsy, To identify relevant genes in urinary exosomes that could predict any-cause rejection, the samples were first analyzed using the TaqMan® OpenArray® Human Inflammation Panel. The panel consists of 586 TagMan assays for genes that have been studied as targets for a range of inflammatory diseases and includes 21 endogenous control assays. In a second analysis, a subset of 112 TagMan assays was identified and plated onto a custom TagMan OpenArray Panel. Given the large number of investigated genes, feature selection was performed on the training data set using Boruta (see Kursa M B, Rudnicki W R. Feature Selection with the Boruta Package. J. Stat Softw 2010; 36(11), incorporated herein by reference in its entirety) to identify 16 relevant genes. To enhance the prediction accuracy and interpretability in generating the rejection probabilities, a LASSO logistic regression model was constructed for the binary outcome of rejection or no rejection.

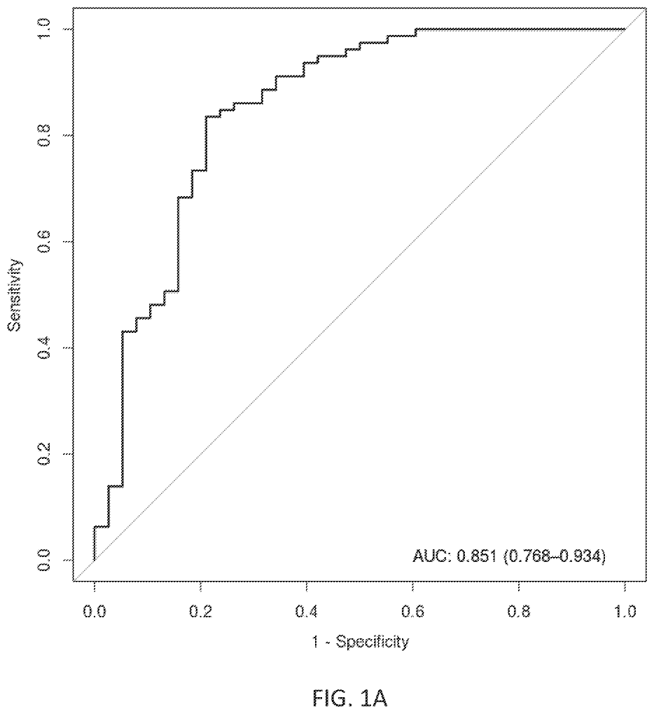

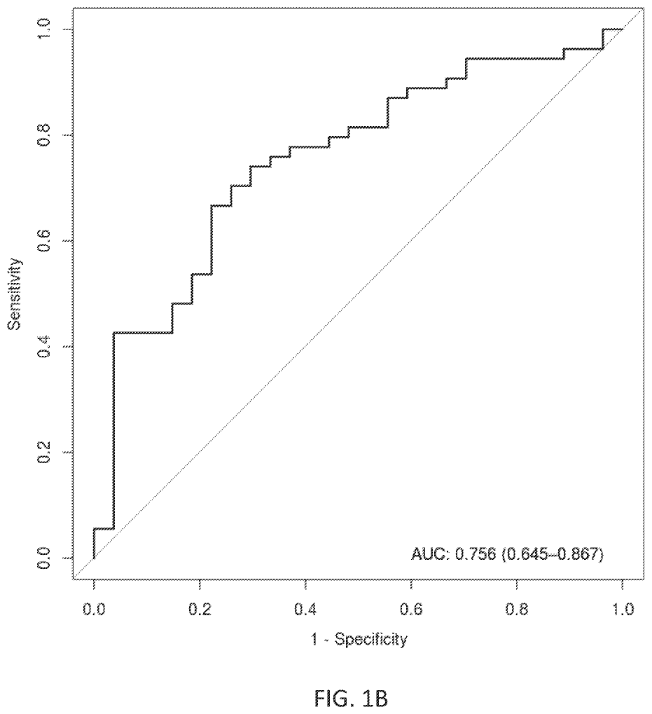

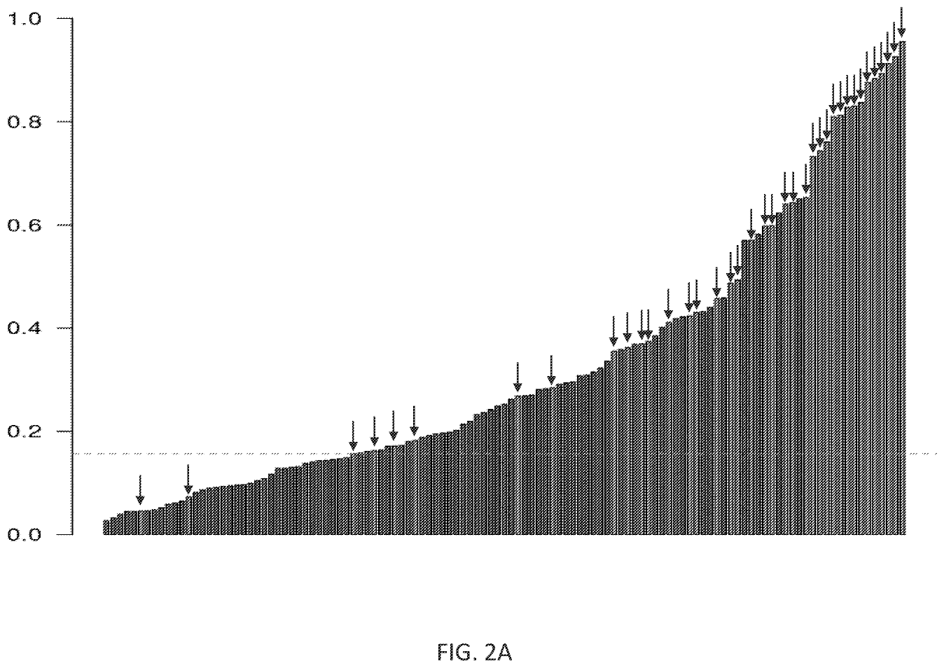

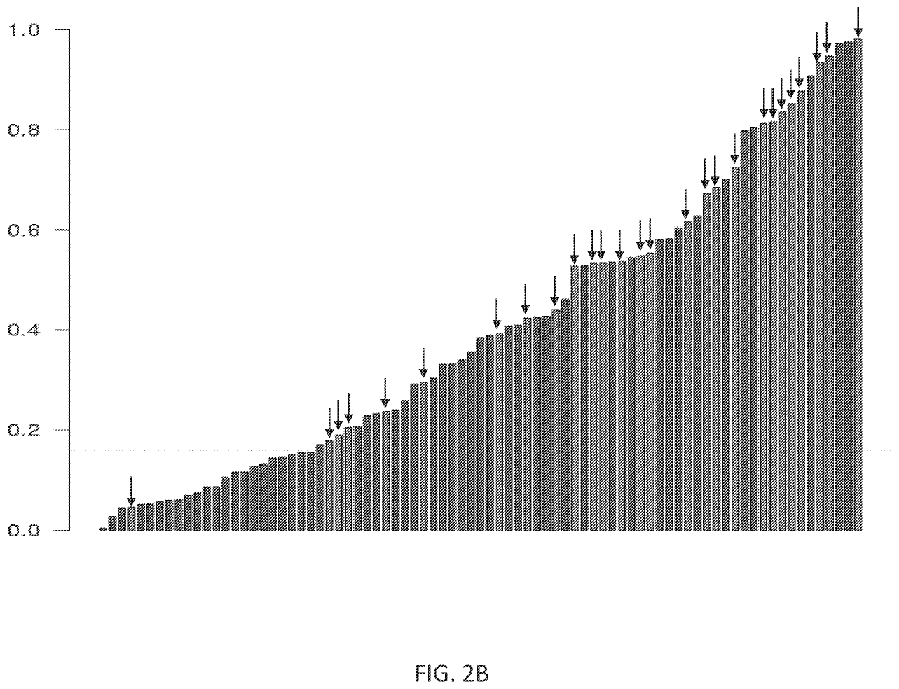

This analysis led to the identification of the 8 gene signature comprising the genes TBP, CXCL10, IFNA4, IL32, UBE2D2, STAT5B, GPI and PYCARD described herein that discriminated biopsies with any-cause rejection from no-rejection in the training and the validation cohorts. The area under the curve-receiver operating characteristics (AUC-ROC) performance was defined for the 8-gene signature in the training and validation data sets. The fraction for true and false positive results for urinary exosome 8-gene signature to discriminate any-cause rejection is shown in FIG. 1A and FIG. 1B. The area under the curve (AUC) was 0.851 (95% CI 0.768 to 0.934) for the training set, as shown in FIG. 1A and 0.756 (95% CI 0.645-0.867) in the validation set, as shown FIG. 1B. The probability of any-cause rejection based on the urinary microvesicular RNA signature for the 190 samples is shown in FIG. 2A and FIG. 2B. Arrows in FIG. 2A and 2B denote samples from biopsy-confirmed kidney rejection patients. A cutoff value for the gene signature that optimized both negative predictive value (NPV) and sensitivity in discriminating biopsies with any-cause rejection from those with no rejection was determined. Using this cutoff point, the gene signature had 94.74% sensitivity (95% CI 82.71 to 98.54%) and 94.44% NPV (95% CI 81,85 to 98.46%) in the training set and 96.3% sensitivity NPV in the validation set. These results indicate that the 8 gene signature comprising TBP, CXCL10, IFNA4, IL32, UBE2D2, STAT5B, GPI and PYCARD can be used to identify patients with any-cause kidney transplant rejection in a method analyzing microvesicular RNA extracted from urinary exosomes.

Identifying a Cell Mediated Kidney Transplant Rejection Gene Signature from Urinary Exosomes.

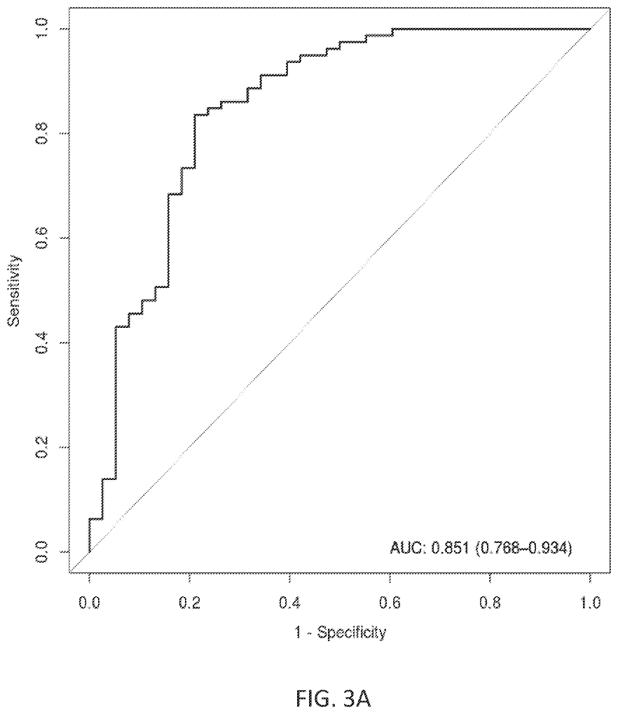

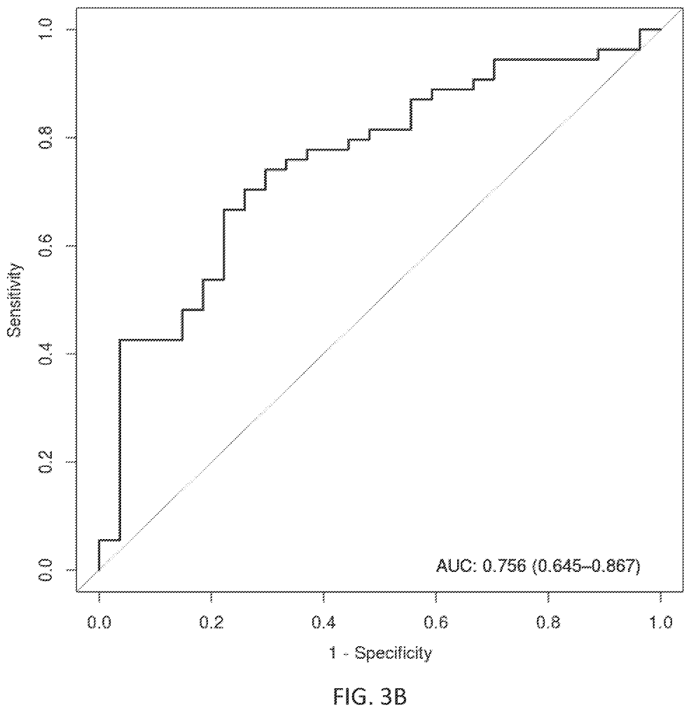

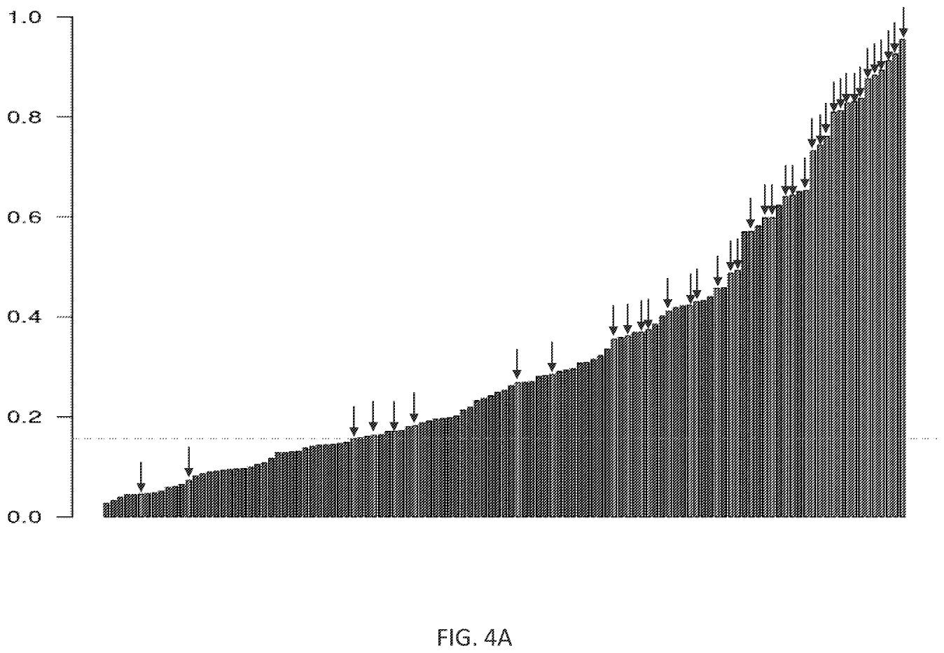

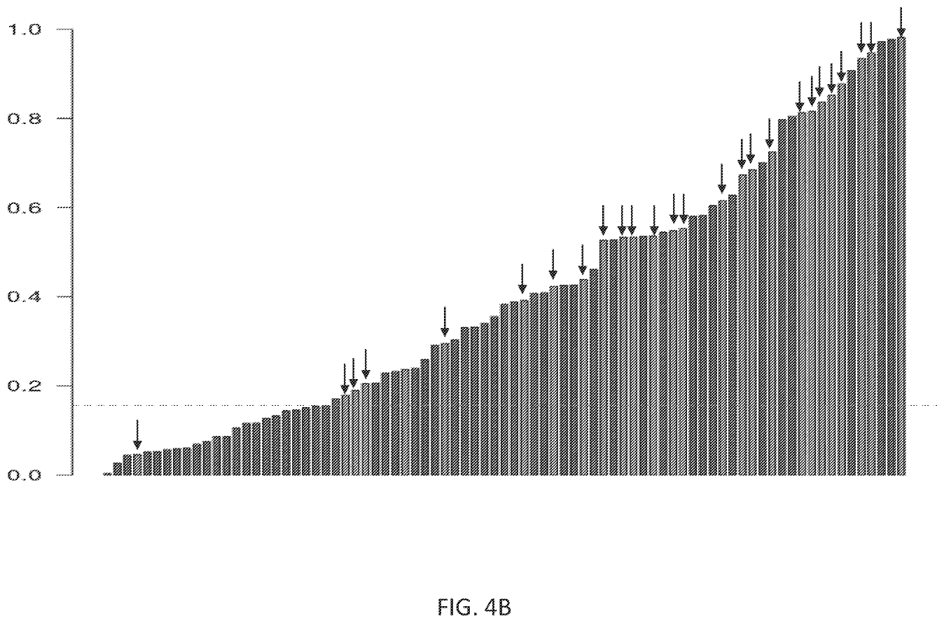

Using the same approach described above, a cell-mediated rejection signature that discriminates between biopsies showing cell-mediated rejection and those showing no-rejection was derived. This analysis led to the identification of the 13 genes signature comprising the genes CXCR4, CD74, HPRT1, CXCL 10, TLR10, IFNA4, UBE2D2, GPL F3, IFNE, FPR2, CXCR2 and IL32 described herein. There was significant overlap to the any-cause rejection signature (5/8). The fractions of true and false positive results for the 13 gene signature to discriminate cell-mediated rejection are shown in FIG. 3A and FIG. 3B for the training and validation sets respectively. The AUC was 0.851 (95% CI 0,768 to 0.934) and 0.756 (95% CI 0.645 to 0.867) for the training and validation sets respectively. FIG. 4A and FIG. 4B show the probability of cell-mediated rejection based on the urinary microvesicular RNA signature in training and validation set. Arrows in FIG. 4A and 4B denote samples from biopsy-confirmed kidney rejection patients. After optimization for NPV and sensitivity, a cutoff point of the 13 genes signature that discriminate biopsies with cell-mediated rejection from no rejection was derived. With this cutoff, the 13-gene signature had an NPV of 96.61% (95% CI 88.46 to 99.07%) in the training set and 94.12% in the validation set. These results indicate that the 13 gene signature comprising CXCR4, CD74, HPRT1, CXCL10, IFNA4, UBE2D2, GPI, F3, IFNE, FPR2, CXCR2, IL32 can he used to identify patients with cell-mediated kidney transplant rejection in a method analyzing microvesicular RNA extracted from urinary exosomes,

Identifying an Any-Cause Kidney Transplant Rejection Gene Signature from Urinary Exosomes

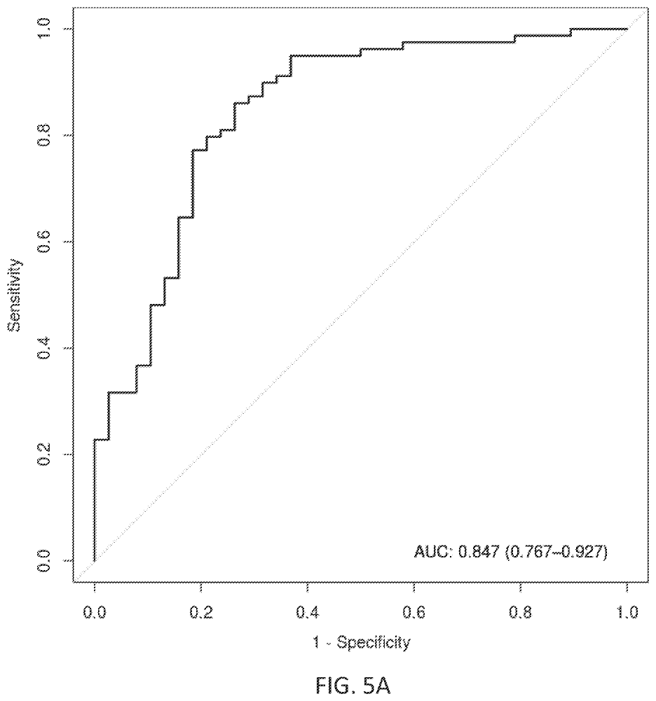

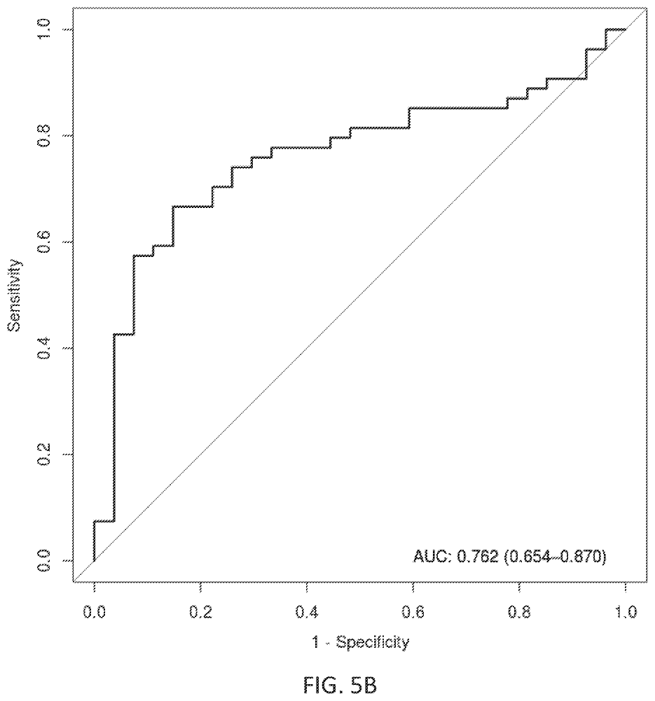

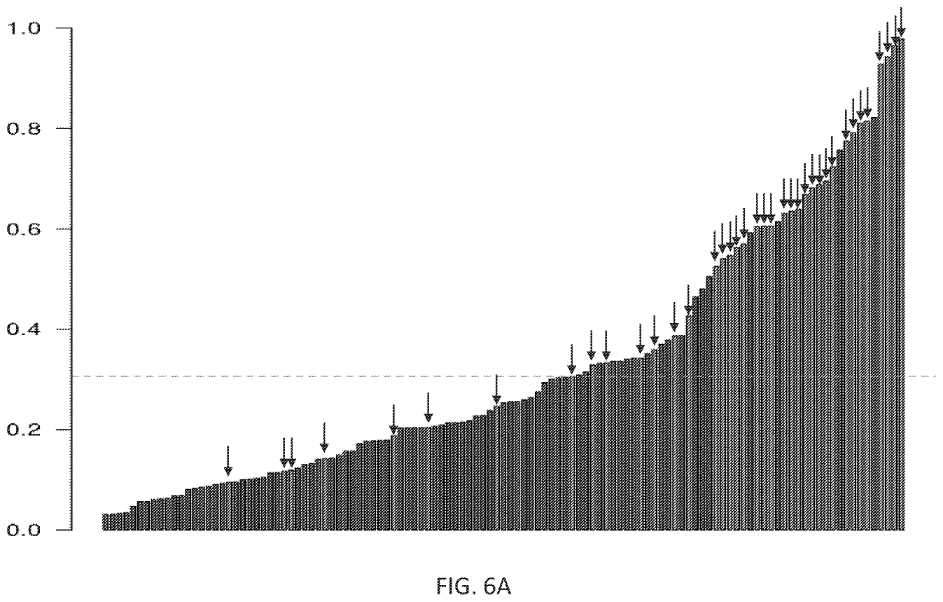

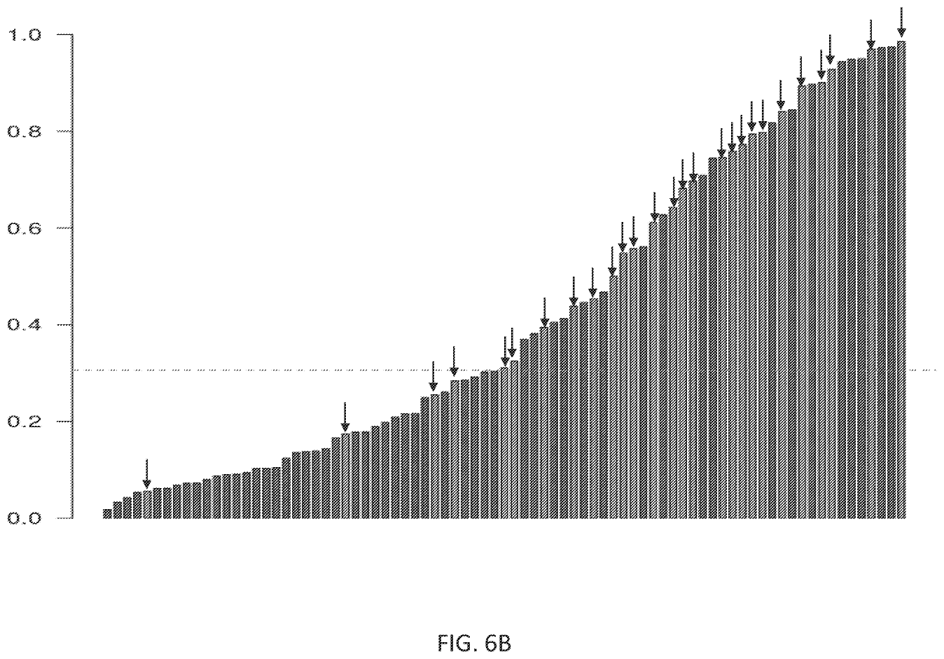

Using the methods described above, an additional 10-gene signature for any-cause kidney transplant rejection was identified. This 10-gene signature comprises the genes CXCL10, IL32, UBE2D2, F3, TBP, NAMPT, CD74, IFNA4, PYCARD and IFNGR1. The fractions of true and false positive results for this 10 gene signature to discriminate any-cause rejection are shown in FIG. 5A and FIG. 5B for the training and validation sets respectively. The AUC was 0.847 (95% CI 0.767-0.927) and 0.762 (95% CI 0.654-0.870) for the training and validation sets respectively. FIG. 6A and FIG. 6B show the probability of any-cause rejection based on the 10-gene signature in the training and validation sets respectively. Arrows in FIG. 6A and 6B denote samples from biopsy-confirmed kidney rejection patients. After optimization for NPV and sensitivity, a cutoff point of the 10-gene signature that discriminate biopsies with any-cause rejection from no rejection was derived. Table 2 shows the NPV, sensitivity, specificity and the PPV for the 10-gene signature when this cutoff value is used. These results indicate that the 10-gene signature comprising CXCL10, IL32, UBE2D2, F3, TBP, NAMPT, CD74, IFNA4, PYCARD, IFNGRI can be used to identify patients with any-cause kidney transplant rejection in a method analyzing microvesicular RNA extracted from urinary exosomes.

TABLE 2

| | Training Set | Validation Set |
|---|---|---|
| NPV | 89.71% | 90.00% |
| Sensitivity | 81.58% | 85.19% |
| Specificity | 77.22% | 66.67% |
| PPV | 63.27% | 56.10% |

Identifying a Cell-Mediated Kidney Transplant Refection Gene Signature from Urinary Exosomes

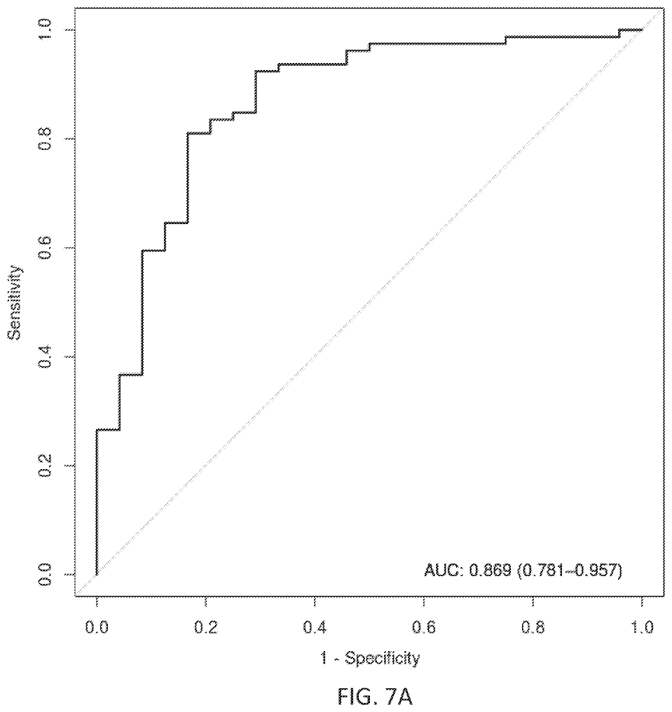

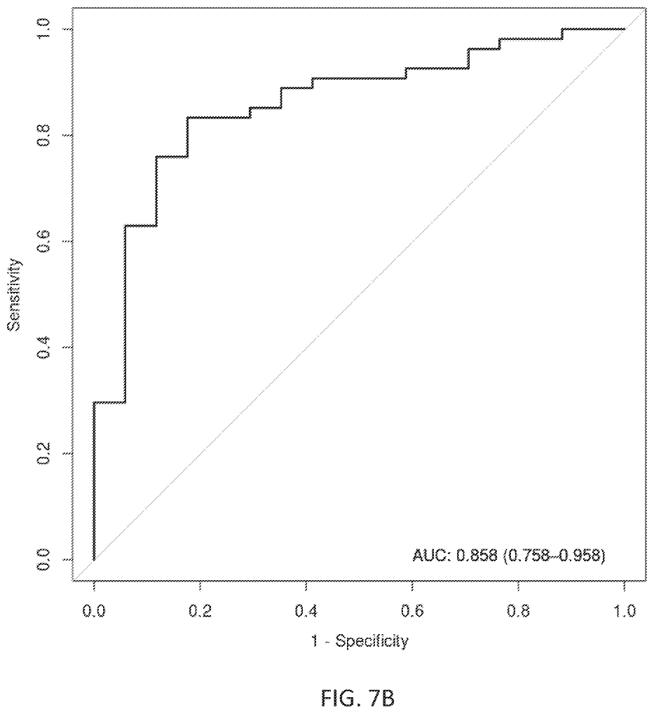

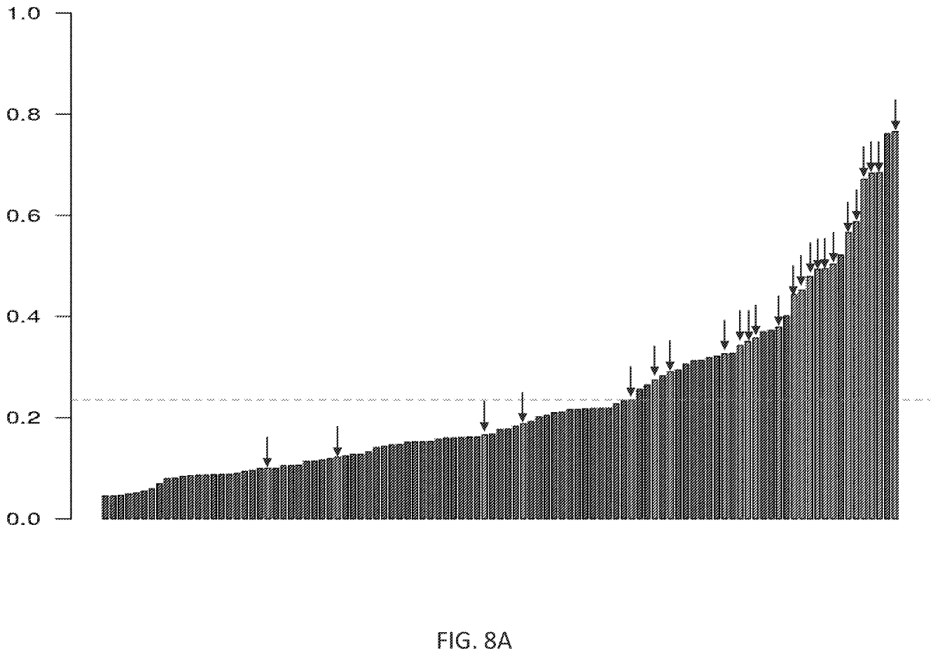

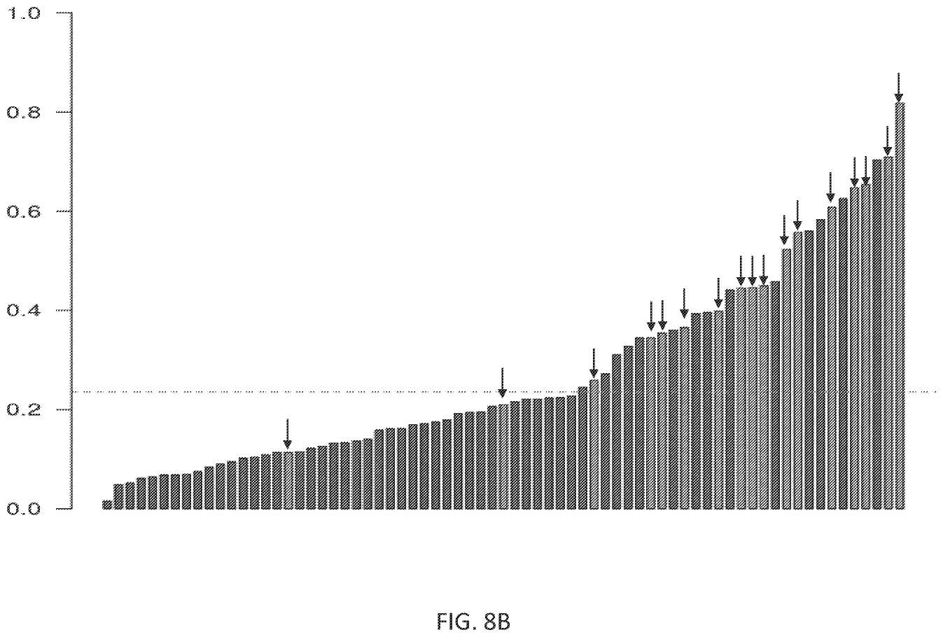

Using the methods described above, an additional 5-gene signature for cell-mediated kidney transplant rejection was identified. This 5-gene signature comprises the genes F3, CD74, CXCL10, UBE2D2 and IFNA4. The fractions of true and false positive results for this 5-gene signature to discriminate cell-mediated rejection are shown in FIG. 7A and FIG. 7B for the training and validation sets respectively. The AUC was 0.869 (95% CI 0.781-0.957) and 0.858 (95% CI 0.758-0.958) for the training and validation sets respectively. FIG. 8A and FIG. 8B show the probability of cell-mediated rejection based on the 5-gene signature in the training and validation sets respectively. Arrows in FIG. 8A and 8B denote samples from biopsy-confirmed kidney rejection patients. After optimization for NPV and sensitivity, a cutoff point of the 5-gene signature that discriminate biopsies with cell-mediated rejection from no rejection was derived, Table 3 shows the NPV, sensitivity, specificity and the PPV for the 5-gene signature when this cutoff value is used. These results indicate that the 5-gene signature comprising F3, CD74, CXCL10, UBE2D2 and IFNA4 can be used to identify patients with cell-mediated kidney transplant rejection in a method analyzing microvesicular RNA extracted from urinary exosomes.

TABLE 3

| | Training Set | Validation Set |
|---|---|---|
| NPV | 94.11% | 95.24% |
| Sensitivity | 83.33% | 88.24% |
| Specificity | 81.01% | 74.07% |
| PPV | 57.14% | 51.72% |

Identifying an Antibody-Mediated Kidney Transplant Rejection Gene Signature from Urinary Exosomes

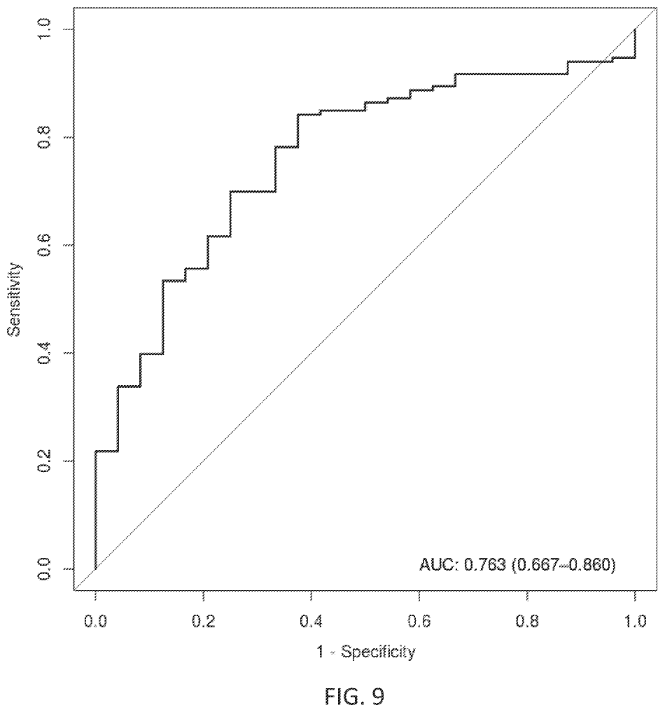

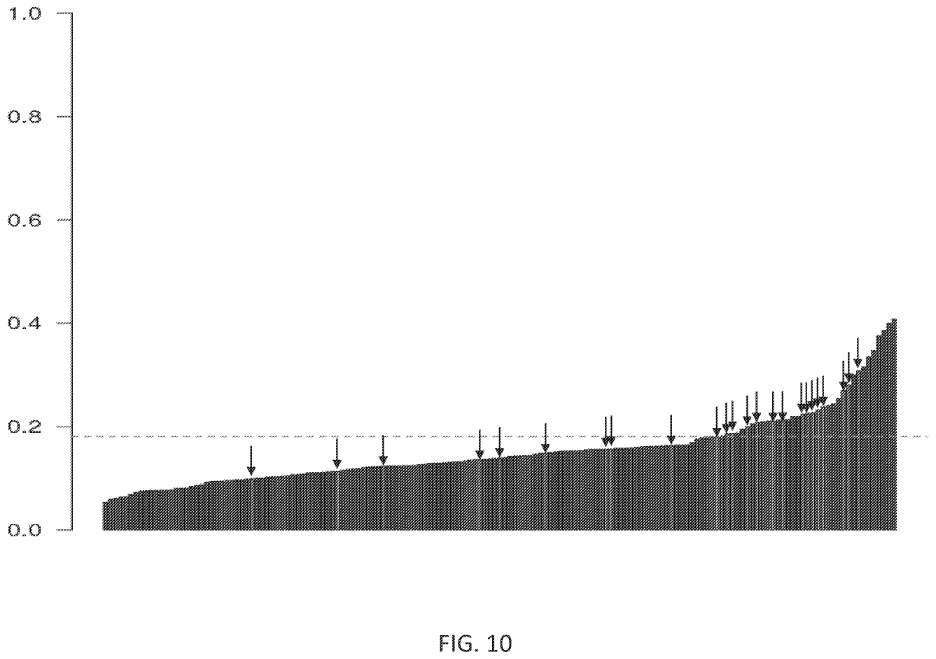

Using the methods described above, an additional 5-gene signature for antibody-mediated kidney transplant rejection was identified. This 5-gene signature comprises the genes HPRT1, CXCR4, CXCL10, IL32 and IFNA4. The fractions of true and false positive results for this 5-gene signature to discriminate antibody-mediated rejection is shown in FIG. 9 for the training set. The AUC was 0.763 (95% CI 0.667-0.860) for the training set. FIG. 10 shows the probability of antibody-mediated rejection based on the 5-gene signature in the training and validation sets respectively. Arrows in FIG. 10 denote samples from biopsy-confirmed kidney rejection patients. After optimization for NPV and sensitivity, a cutoff point of the 5-gene signature that discriminate biopsies with antibody-mediated rejection from no rejection was derived, Table 4 shows the NPV, sensitivity, specificity and the PPV for the 5-gene signature when this cutoff value is used. These results indicate that the 5-gene signature comprising HPRT1, CXCR4, CXCL10, IL32 and IFNA4 can be used to identify patients with antibody-mediated kidney transplant rejection in a method analyzing microvesicular RNA extracted from urinary exosomes.

TABLE 4

| | Training Set |
|---|---|
| NPV | 92.56% |
| Sensitivity | 62.50% |
| Specificity | 84.21% |
| PPV | 41.67% |

### Example 2

A total of 192 urine samples that have matched biopsy specimens were analyzed in the following example to derive the methods described in the present disclosure.

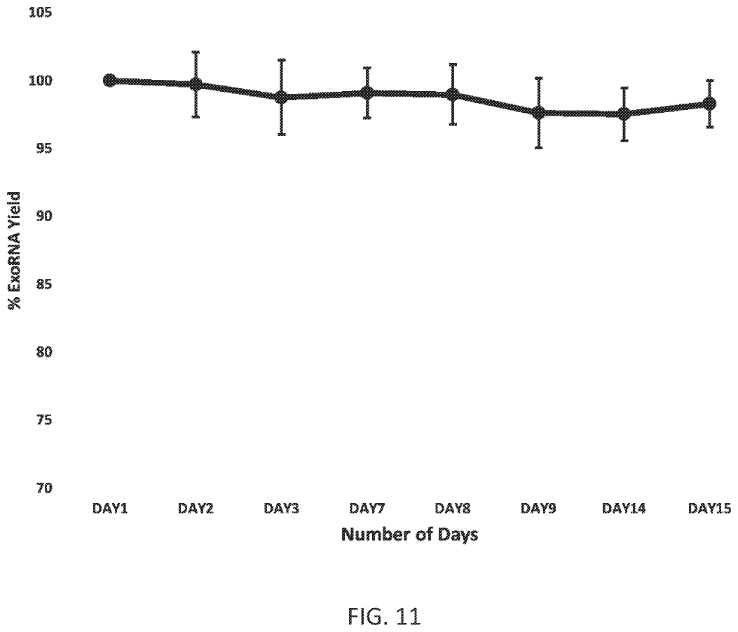

As shown in FIG. 11, exosomal mRNA showed stability in urine stored at 4° C. for 2 weeks. Without wishing to he bound by theory, the stability of mRNA is critical for developing clinically useful diagnostic tests as the samples can he safely cold-pack shipped from patient's residence to a central laboratory for analysis, where they can be either processed immediately or stored at ±4° C. for up to 2 weeks. Urine samples were collected and stored at 4° C. for up to two weeks. Exosomes were extracted at different time-points followed by qRT-PCR to analyze the yield and integrity of the RNA. The urine exosome RNA was stable over two weeks (average yield from three separate genes). The error bars in FIG. 11 represent the standard deviation of the percentage of exosomal RNA yield across three different genes.

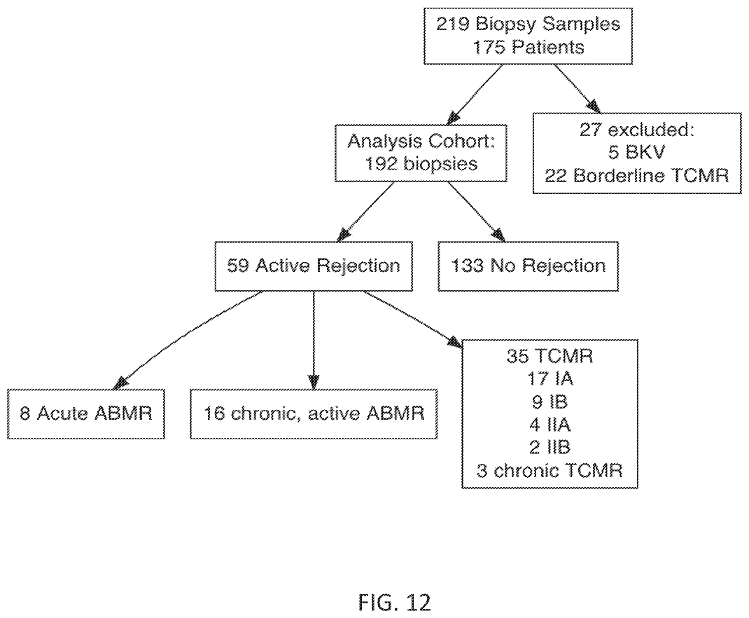

The following analysis included matched urine samples for biopsy specimens showing TCMR (Grades IA, IB, IIA, IIB), acute active and chronic active ABMR sub-groups rejection, based on the Banff classification and used the term active rejection to distinguish them from other biopsy specimens without rejection. There were 59 biopsy specimens with, and 133 biopsy specimens without active rejection (30.7% prevalence). FIG. 12 shows the results for the 192 biopsies that had matched urine samples. Table 5 shows the baseline characteristics of the study cohorts. The mean age of patients with any-cause rejection was 51.0 [38.0-64.5] years and 51.6 [40.8-65] in patients without rejection. Median estimated glomerular filtration rate (eGFR) levels were 32.85 [22.13-44.56] in patients with any-cause rejection and 37.89 [25.95-50.89] in patients with no rejection. The any-cause rejection group included a higher proportion of patients with previous rejection episodes (p=8.2 3e-07) and longer time since biopsy when compared to the group without rejection (p=0.02). The difference in the proportion of black patients between the groups was not significant (p=0.47). Among the any-cause rejection group, 59.3% of rejection cases were due to acute TCMR, and 40.7% attributed to ABMR.

TABLE 5

| | Clinical Cohort (n = 192) | | | | |
|---|---|---|---|---|---|
| | No | | Any-Cause | | |
| | Rejection | | Rejection | | |
| Characteristic | (n = 133) | | (n = 59) | | p-value |
| Age, years | 51.6 ± 15.1 | | 51.0 ± 16.2 | | 0.80\hat{ } |
| Female, % | 32.3 | | 45.8 | | |
| Race, % | | | | | |
| White | 83.6 | | 88.0 | | 0.47^{#} |
| Black | 16.4 | | 22.0 | | 0.47^{#} |
| SCr at Biopsy, | 1.8 | [1.5-2.6] | 2.2 | [1.7-2.8] | 0.39\hat{ } |
| mg/dl | | | | | |
| eGFR | 37.9 | [25.6-50.9] | 32.9 | [22.1-44.6] | 0.02\hat{ } |
| Previous | 15.2 | | 42.4 | | 8.23 e−07^{#} |
| Rejection, % | | | | | |
| Deceased | 43.0 | | 51.9 | | 0.65^{#} |
| Donor, % | | | | | |
| Time to | 215 | [46-1751] | 1250 | [295-3063] | 0.02\hat{ } |
| Biopsy (days) | | | | | |

| | | | |
|---|---|---|---|
| Thymoglobulin | 60.5 | 69.4 | 0.36 |
| % | | | |
| Rejection | — | | — |
| Type, % | | | |
| Cellular | | 59.3 | |
| Antibody | | 40.7 | |
| Mediated | | | |
| Banff | | | — |
| classification % | | | |
| IA | | 42.9 | |
| IB | | 20.0 | |
| 2A | | 8.6 | |
| 2B | | 7 | |

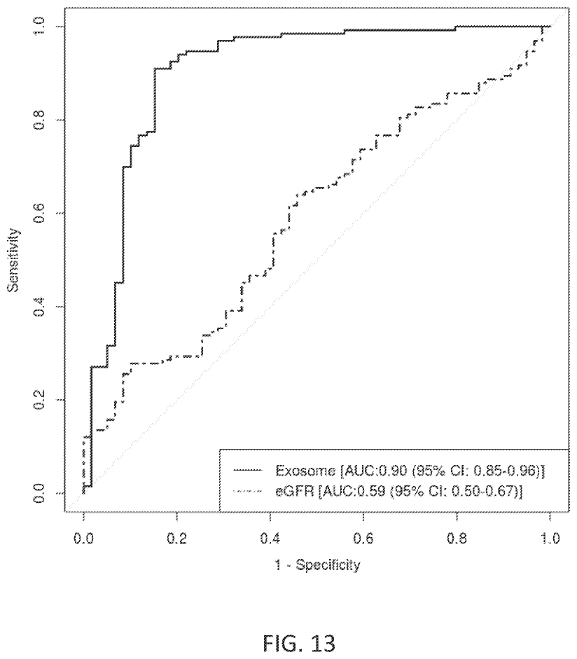

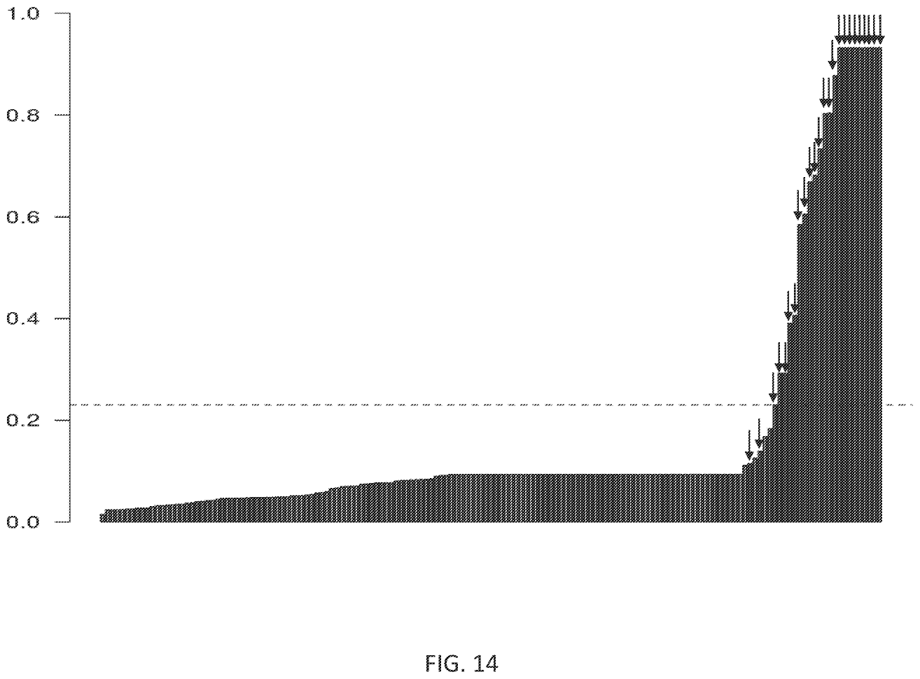

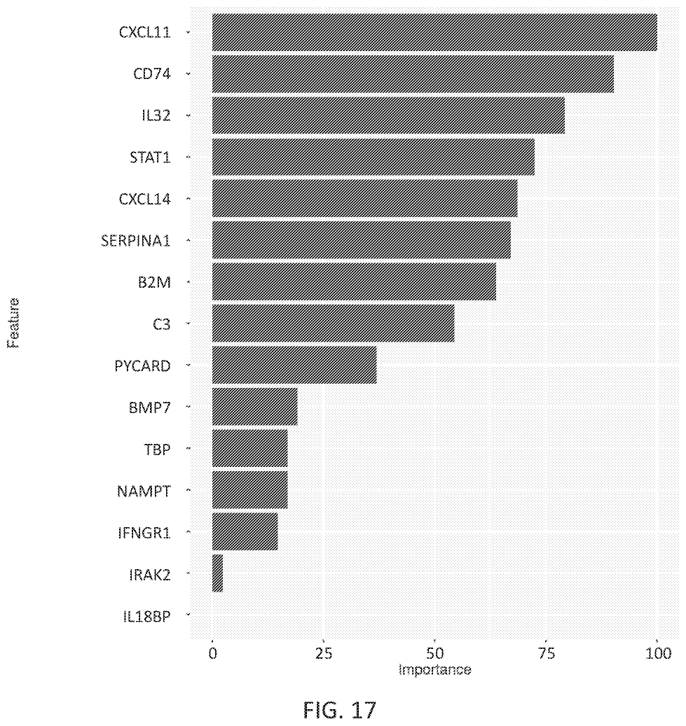

mRNA from urinary exosomes in urine samples collected from patients with biopsy proven any-cause rejection were compared to urine samples from patients without rejection. In order to identify relevant genes in urinary exosomes that could predict any-cause rejection, the samples were first analyzed using the TaqMan® OpenArray® Human Inflammation Panel. This panel consists of 586 TaqMan assays for genes that have been studied as targets for a range of inflammatory diseases and includes 21 endogenous control assays. For subsequent analyses, a subset of 112 TaqMan assays was identified and plated onto a custom TagMan OpenArray Panel. Given the large number of investigated genes, feature selection was performed using Bonita to identify the relevant features. A repeated stratified K-fold classification model (k=10, repeats=10) with a support vector machine (SVM) using a radial basis function (RBF) kernel was used for classification. The stratification ensures that there is a similar percentage of samples with rejection in each of the folds. This process is repeated ten times with a different randomization in each repeat to generate the final classification model. Without wishing to be bound by theory, cross-validation was used instead of hold-out because cross-validation improves the generalizability of the gene signature by validating the performance on multiple train-test subsets of the data and results in a much more stable estimate of the performance. This allowed for the identification of a multi-gene signature (CXCL11, CD74, IL32, STAT1, CXCL14, SERPINA1, B2M, C3, PYCARD, BMP7, TBP, NAMPT, IFNGR1, IRAK2, IL18BP) that discriminated biopsies with any-cause rejection from no-rejection. As shown in FIG. 13, the area under the curve (AUC) was 0.90 (95% CI 085-0.96). In order to compare the performance of this signature against current clinical practice, an AUC for estimated glomerular filtration rate (eGFR) was also generated. As shown in FIG. 13, the AUC for eGFR for this set of patients was 0.59 (95% CI 0.50-0.67), which was significantly inferior (p:=1.62 e-09) to the performance of the multi-gene signature. As shown in FIG. 14, a cutoff value to rule out any-cause rejection was also derived using by optimizing Youden's J. This resulted in an NPV of 93.1% (95% CI 87.4%-96.3%) and a sensitivity of 84.7% (95% CI 73.5%-91.8%). The PPV for discriminating active rejection was 80.6% (95% CI 69.1%-88.6%) (see Table 6). FIG. 17 shows the analysis of each gene in the signature to determine the relative importance of each gene in the signature.

TABLE 6

| | Performance (95% CI) |
|---|---|
| NPV | 93.1% (87.4%-96.3%) |
| Sensitivity | 84.7% (73.5%-91.8%) |
| Specificity | 91.0% (84.9%-94.8%) |
| PPV | 80.6% (69.1%-88.6%) |

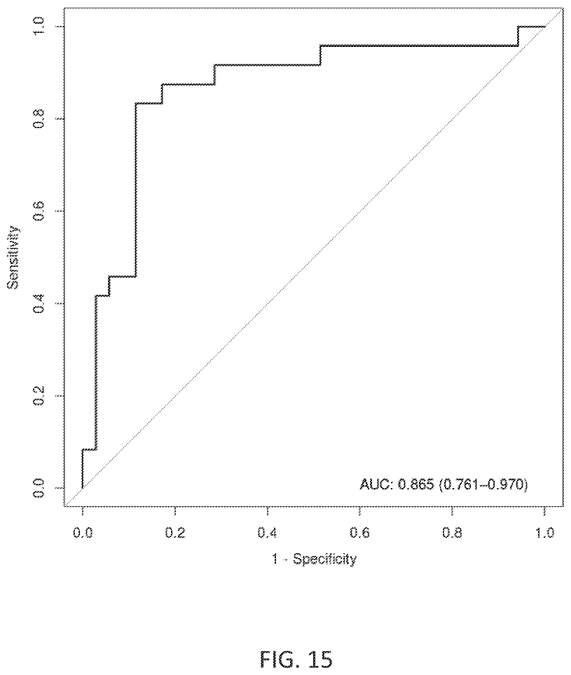

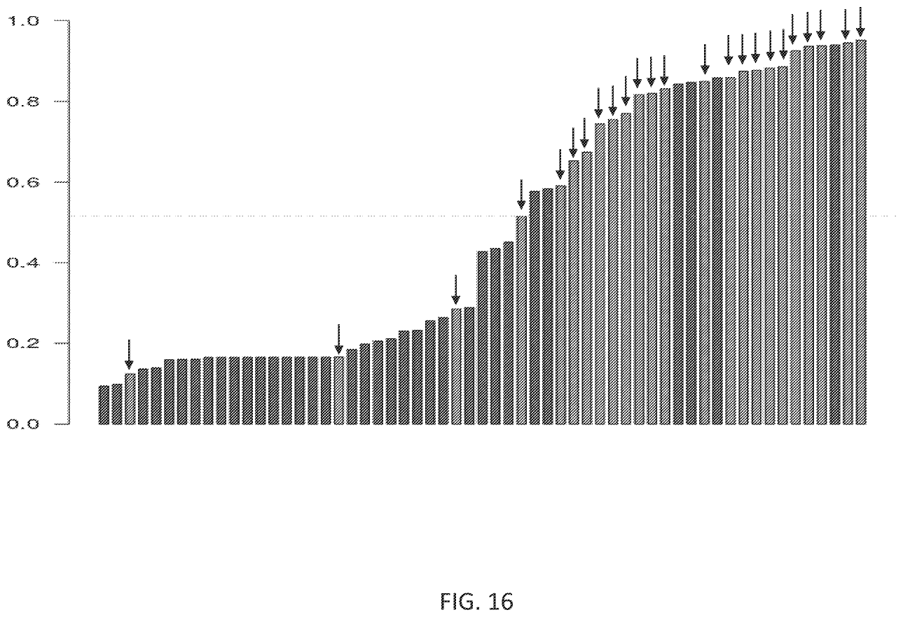

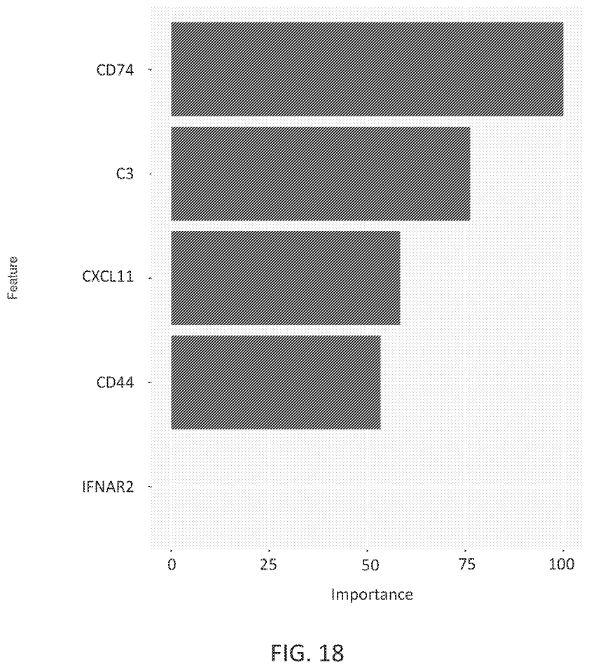

TCMR (t-cell mediated rejection) samples were also compared to the ABMR (antibody-mediated rejection) samples to derive an additional signature to discriminate between these two forms of rejection. Applying the same optimization and classification approach used for any-cause rejection, a multi-gene signature (CD74, C3, CXCL11, CD44, IFNAR2) was identified that could distinguish TCMR. from ABMR. As shown in FIG. 15, the AUC for this signature was 0.87 (95% CI 0.76-0.97). As shown in FIG. 16, a cutoff value was derived to maximize the NPV and sensitivity to rule out antibody-mediated rejection. This resulted in an NPV of 90.6% (95% CI 75.8%-96.8%) and a PPV of 77.8% (95% CI 59.2%-89.4%). The sensitivity to discriminate TCMR from ABMR was 87.5% (95% CI 69.0%-95.7%) and the specificity was 82.9% (95% CI 67.3%-92.0%) (see Table 7). FIG. 18 shows the analysis of each gene in the signature to determine the relative importance of each gene in the signature.

TABLE 7

| | Performance (95% CI) |
|---|---|
| NPV | 90.6% (75.8%-96.8%) |
| Sensitivity | 87.5% (69.0%-95.7%) |
| Specificity | 82.9% (67.3%-92.0%) |
| PPV | 77.8% (59.2%-89.4%) |

Materials and Methods

Patient and Sample Information

175 kidney transplant patients were enrolled at the time of a clinically indicated renal biopsy from 3 renal centers. A total of 219 urine samples were collected from patients for urinary exosomal mRNA profiling. Demographic and clinical characteristics and information on the donors were collected from the medical chart. The on-site pathologist's renal transplant biopsy report was used to define active rejection in accordance with the Banff Working Groups criteria, Samples that were diagnosed as borderline cell mediated rejection or BK virus nephropathy were excluded. For the analysis described above, TCMR, acute active and chronic active ABMR were integrated to form the active rejection group and distinguished them from samples that were classified as having no rejection based on biopsy reports. Biopsy reports with diagnosis of mixed ABMR and TCMR were grouped with the TCMR subgroup and those with mixed borderline TCMR and ABMR were grouped with the ABMR subgroup.

Urinary Exosome Isolation, mRNA Extraction and Gene Expression Analysis

The second voided urine sample was collected on the morning of the biopsy, and the urine samples were stored at −80° C. Three in-house controls were used, consisting of 1 pooled male sample. I pooled female sample, and 1 pooled male & female sample. Up to 20 ml urine were centrifuged to remove cells and cellular debris at 2000×g for 20 minutes. Exosomes were isolated from the urine supernatant using a urine exosome isolation kit. RNA was eluted in 16 μl nuclease-free H₂O, 14 μl of which was used in a 20 μl reverse transcription (RT) reaction using the VILO cDNA synthesis kit (Thermo Fisher).

The first round of samples was analyzed using the TaqMan ® OpenArray® Human Inflammation Panel (Thermo Fisher). This panel consists of 586 TaqMan assays for genes that have been studied as targets for a range of inflammatory diseases and includes 21 endogenous control assays. To prepare the samples for quantitative PCR (qPCR), 10 μl cDNA was split into two, equal portions and pre-amplified with two pools of mixed primers following the manufacturer's directions. The pre-amplification reactions were mixed and diluted prior to mixing with TaqMan® OpenArray® Real-Time PCR Master Mix. Reaction mixes were loaded onto the OpenArray plates and the plates run on the QuantStudio™ 12K Flex real-time PCR system (Thermo Fisher) using the preset protocol for this panel.

Based on the initial analysis, a subset of assays was identified and plated onto a custom TaqMan OpenArray Panel. This panel consisted of 112 TaqMan assays. For this panel, 5 cDNA was pre-amplified with a pool of the 112 assays using the manufacturer's directions. The pre-amplification reactions were diluted prior to mixing samples TaqMan® OpenArray® Real-Time PCR Master Mix, Reaction mixes were loaded onto the OpenArray plates and the plates run on the QuantStudio™ 12K Flex real-time PCR system (Thermo Fisher) using the preset protocol for this panel. Analysis of samples described here used the 112 TaqMan assays common to all samples.

Statistical Analyses

Genes with data missing from >20% of the samples were excluded from the analysis. Missing data was imputed using a non-parametric missing value imputation. The Ct values from the OpenArray were normalized to PGK1. The Boruta algorithm was used for feature selection. An SVM with a radial kernel was fit to the relevant features using a repeated K-fold cross-validation (K=10, repeats=10) to generate the rejection probabilities using the caret package. This approach gives a better indication of how well the model will perform on unseen data compared to just one train-test split in a hold-out method that makes it highly dependent on how the data is split in test and train datasets. The pROC package was used to generate the ROC curves. Associations between clinical and demographic factors were computed using Student's t-test for continuous variables and Pearson's Chi-Squared test for categorical variables. AUC comparison was performed using DeLong's test. Data reporting and analyses were conducted using R version 3.3. Two-tailed p-values 0.05 were considered statistically significant. Sample size was calculated for an NPV and specificity of 90% with a 10% width for the 95% CI at a prevalence of 30%. Based on this calculation, the required sample size was estimated to be 116 samples.

### Example 3

Using the analysis described in Example 2, a gene signature for the identification of cell-mediated kidney transplant rejection was derived (CD74, CXCL11, C3, CCL2, B2M, IL15, IL18BP, FPR2, ALOX5AP, IL1RAP, TLR1, NAMPT and IL1R2).

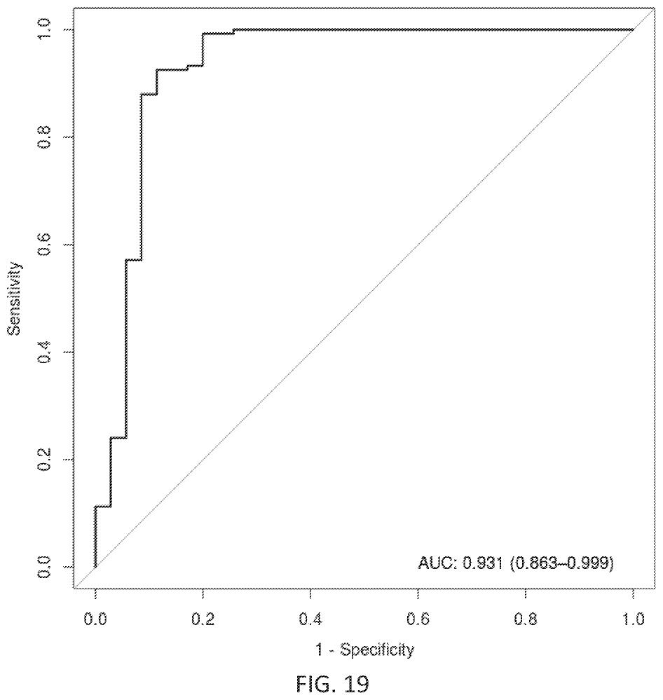

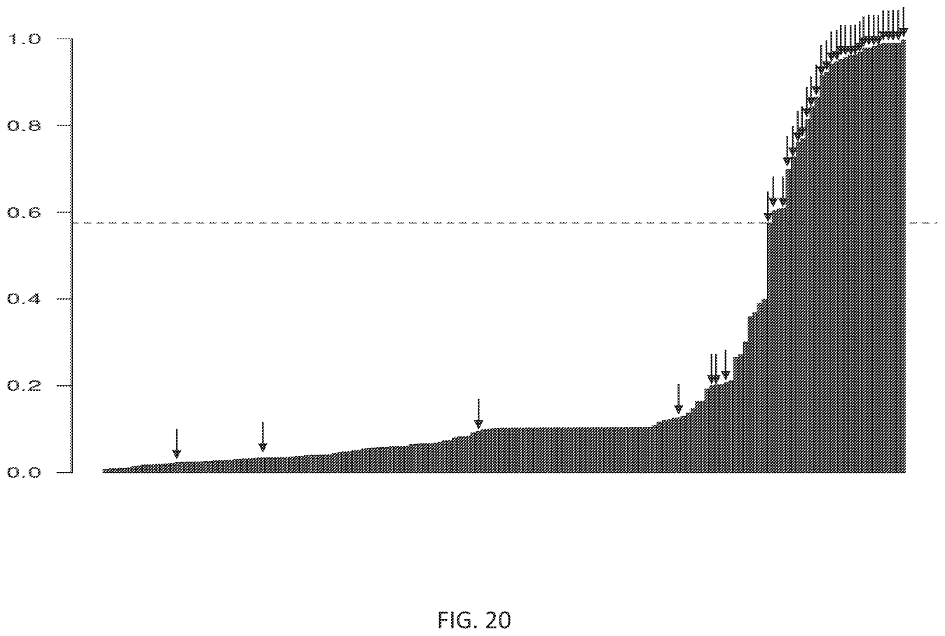

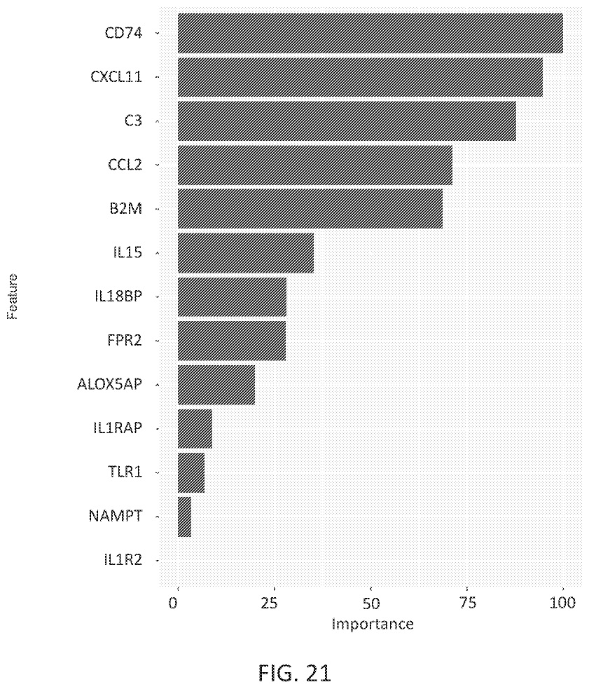

As shown in FIG. 19, the AUC for this signature was 0.931 (95% CI 0.863-0.99). As shown in FIG. 20, a cutoff value was derived to maximize the NPV and sensitivity to identify cell-mediated kidney transplant rejection This resulted in an NPV of 95.0% (95% CI 90.0-97.5) and a PPV of 96.6% (95% CI 82.8-99.4), The sensitivity to identify cell-mediated kidney transplant rejection was 80.0% (95% CI 64.1-90.0) and the specificity was 99.3% (95% CI 95.9-99.9) (see Table 8). FIG. 21 shows the analysis of each gene in the signature to determine the relative importance of each gene in the signature.

TABLE 8

| | Performance |
|---|---|
| NPV | 95.0% (90.0-97.5) |
| Sensitivity | 80.0% (64.1-90.0) |
| Specificity | 99.3% (95.9-99.9) |
| PPV | 96.6% (82.8-99.4) |

### Example 4

Using the analysis described in example 2, a gene signature for the identification of antibody-mediated kidney transplant rejection was derived (CD44, NAMPT, PYCARD, IRAK2, IL32, TBP, BCL10, IFNGR1, BMP7, STAT1, ANXA1, TYMP, NFX1).

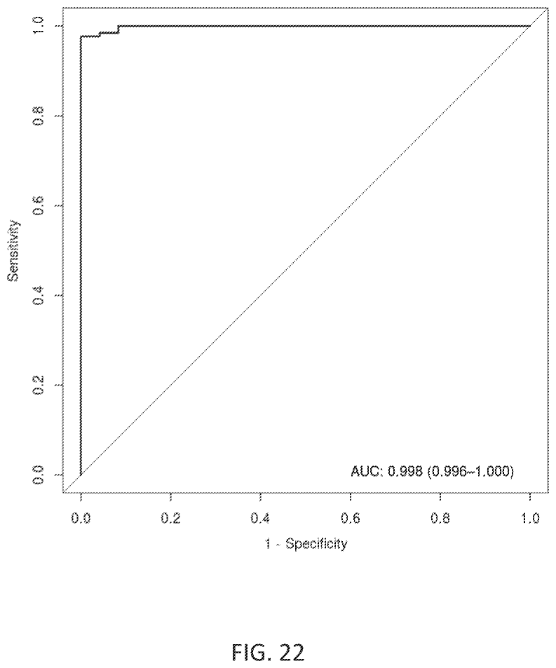

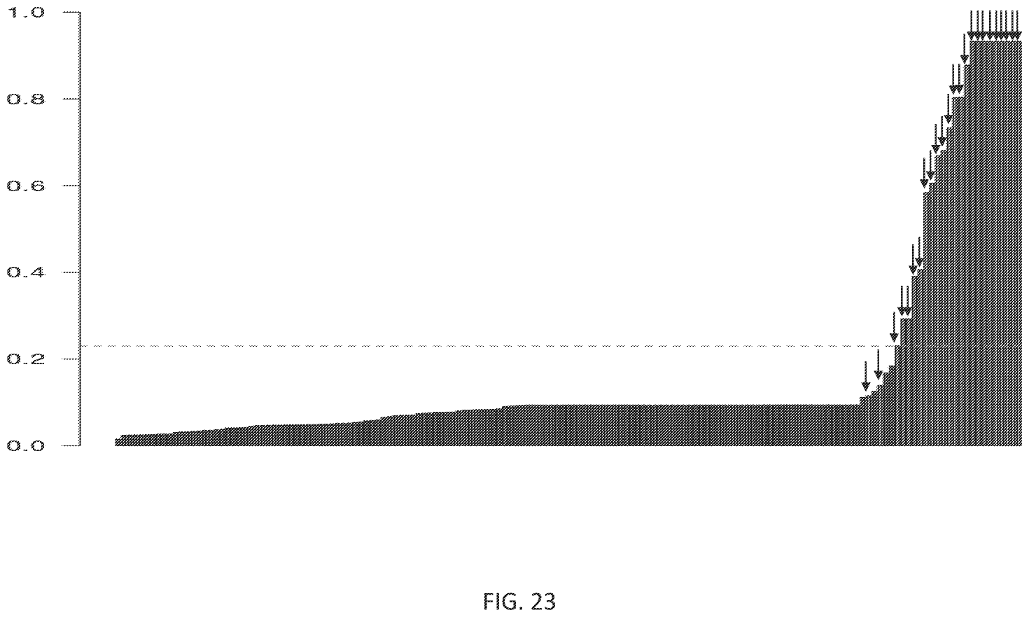

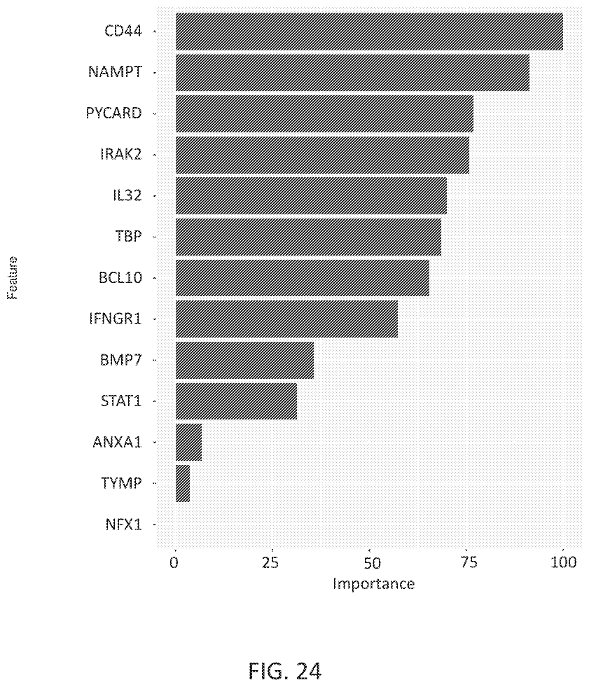

As shown in FIG. 22, the AUC for this signature was 0.998 (95% CI 0.996-1.000). As shown in FIG. 23, a cutoff value was derived to maximize the NPV and sensitivity to identify antibody-mediated kidney transplant rejection This resulted in an NPV of 98.5% (95% CI 94.8-99.6) and a PPV of 100.0% (95% CI 85.1-100.0), me sensitivity to identify antibody-mediated kidney transplant rejection was 91.7% (95% CI 74.2-97.7) and the specificity was 100.0% (95% CI 97.2-100.0) (see Table 9). FIG. 24 shows the analysis of each gene in the signature to determine the relative importance of each gene in the signature.

TABLE 9

| | Performance | |
|---|---|---|
| NPV | 98.5% | (94.8-99.6) |
| Sensitivity | 91.7% | (74.2-97.7) |
| Specificity | 100.0% | (97.2-100.0) |
| PPV | 100.0% | (85.1-100.0) |

The following is a non limiting example demonstrating that the gene signatures of the present disclosure can be measured and used to identify kidney transplant rejection and the risk of kidney transplant rejection using low sample volumes of urine.

The expression level of the biomarkers from the signature derived to distinguish any-cause kidney transplant rejection from no kidney transplant rejection (CXCL11, CD74, IL32, STAT1, CXCL14, SERPINA1, B2M, C3, PYCARD, BMP7, TBP, NAMPT, IFNGR1, IRAK2, IL18BP) in Example 2, the biomarkers from the signature derived to distinguish TCMR from ABMR (CD74, C3, CXCL11, CD44, IFNAR2) in Example 2, and the reference biomarker PGK1, were measured in microvesicular RNA isolated from urine samples of 3 ml, 5 ml, 10 ml and 20 ml using quantitative PCR. Table 10 shows the average et value measured for each of the genes in each of the sample volumes and the linearity between the different sample volumes.

TABLE 10

| | Sample Volume | | | | |
|---|---|---|---|---|---|
| Biomarker | 20 mL | 10 mL | 5 mL | 3 mL | Linearity |
| BMP7 | 23 | 24 | 25.2 | 25.1 | 0.952 |
| IL32 | 17.6 | 18.2 | 19.5 | 19.7 | 0.897 |
| IRAK2 | 20.4 | 21.4 | 22.3 | 22.7 | 0.974 |
| IFNGR1 | 21.2 | 21.8 | 23.5 | 23.5 | 0.864 |
| IFNAR2 | 21 | 21.6 | 23 | 23.1 | 0.889 |
| IL18BP | 24.1 | 24.9 | 25.5 | 25.7 | 0.991 |
| PGK1 | 15.6 | 16.3 | 17.4 | 17.7 | 0.929 |
| SERPINA1 | 17.7 | 18.3 | 19.6 | 19.7 | 0.898 |
| CXCL14 | 17.7 | 18.4 | 19.7 | 19.8 | 0.915 |
| CD74 | 17.9 | 18.7 | 20 | 20.8 | 0.891 |
| NAMPT | 16.4 | 17.1 | 18.3 | 18.6 | 0.920 |
| CD44 | 19.2 | 19.9 | 21.2 | 21.2 | 0.913 |
| B2M | 15.6 | 16.3 | 17.8 | 17.7 | 0.891 |
| CXCL11 | 22.5 | 23.3 | 25.2 | 25.3 | 0.887 |
| STAT1 | 17.3 | 18 | 19.2 | 19.3 | 0.925 |
| PYCARD | 21.8 | 22.5 | 24 | 24.2 | 0.899 |
| TBP | 19.7 | 20.5 | 21.9 | 22 | 0.922 |
| C3 | 23.2 | 24.2 | 25.7 | 26.3 | 0.924 |

As shown in Table 10, there was strong correlation between the expression levels measured in the different sample volumes. Without wishing to be bound by theory, these results indicate that the gene signatures of the present disclosure are robust enough to be used in situations where only low sample volumes of urine can be obtained from a subject,

What is claimed is:

1. A method of determining the risk of a kidney transplant rejection in a subject who has undergone a kidney transplant, the method comprising:
a) determining the expression level of at least two of 15 biomarkers in microvesicular RNA isolated from a biological sample from the subject, wherein the 15 biomarkers comprise CXCL11, CD74, IL32, STAT1, CXCL14, SERPINA1, B2M, C3, PYCARD, BMP7, TBP, NAMPT, IFNGR1, IRAK2 and IL18BP;
b) inputting the expression levels from step (a) into an algorithm to generate a score; and
c) determining the risk of a kidney transplant rejection in the subject based on the score.

2. The method of claim 1, wherein the kidney transplant ejection is any-cause kidney transplant rejection.

3. The method of any one of the preceding claims, wherein step (a) comprises determining the expression level of:
a) at least three of the 15 biomarkers;
b) at least four of the 15 biomarkers;
c) at least five of the 15 biomarkers;
d) at least six of the 15 biomarkers;
e) at least seven of the 15 biomarkers,
f) at least eight of the 15 biomarkers:
g) at least nine of the 15 biomarkers;
h) at least ten of the 15 biomarkers;
i) at least 11 of the 15 biomarkers,
j) at least 12 of the 15 biomarkers;
k) at least 13 of the 15 biomarkers; or
l) at least 14 of the 15 biomarkers.

4. The method of any one of the preceding claims, wherein step (a) comprises determining the expression level of each of the 15 biomarkers.

5. The method of any one of the preceding claims, wherein determining the risk of a kidney transplant rejection in the subject based on the score comprises:
i) comparing the score to a predetermined cutoff value; and
ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

6. A method of determining the risk of an antibody-mediated kidney transplant rejection as opposed to a cell-mediated kidney transplant rejection in a subject who has undergone a kidney transplant and has been identified as having a kidney transplant rejection and/or identified as being at high risk of having a kidney transplant rejection, the method comprising:
a) determining the expression level of at least two of five biomarkers in microvesicular RNA isolated from a biological sample from the subject, wherein the five biomarkers comprise CD74, C3, CXCL11, CD44 and IFNAR2;
b) inputting the expression levels from step (a) into an algorithm to generate a score; and
c) determining the risk of an antibody-mediated kidney transplant rejection as opposed to a cell-mediated kidney transplant rejection in the subject based on the score.

7. The method of claim 6, wherein step (a) comprises determining the expression level of:
a) at least three of the five biomarkers; or
b) at least four of the five biomarkers.

8. The method of claim 7, wherein step (a) comprises determining the expression level of each of the five biomarkers.

9. The method of claims 6-8, wherein determining the risk of an antibody-mediated kidney transplant rejection as opposed to a cell-mediated kidney transplant rejection in the subject based on the score comprises:
i) comparing the score to a predetermined cutoff value; and
ii) determining that the at the subject is at a higher risk of having an antibody-mediated kidney transplant rejection as opposed to a cell-mediated kidney transplant rejection when the score is greater than or equal to the predetermined cutoff value, or determining that the subject is at a higher risk of having a cell-mediated kidney transplant rejection when the score is less than the predetermined cutoff value.

10. A method comprising:
i) performing the method of any one of claims 1-5; and
ii) when the subject is identified as being at risk for a kidney transplant rejection, performing the method of any one of claims 6-9.

11. A method of determining the risk of a cell-mediated kidney transplant ejection in a subject who has undergone a kidney transplant, the method comprising:
a) determining the expression level of at least two of 13 biomarkers in microvesicular RNA isolated from a biological sample from the subject, wherein the 13 biomarkers comprise CD74, CXCL11, C3, CCL2, B2M, IL 15, IL18BP, FPR2, ALOX5AP, IL1RAP, TLR1, NAMPT and IL1R2;
b) inputting the expression levels from step (a) into an algorithm to generate a score; and
c) determining the risk of a cell-mediated kidney transplant rejection in the subject based on the score.

12. The method of claim 11, wherein determining the risk of a cell-mediated kidney transplant rejection in the subject based on the score comprises:
i) comparing the score to a predetermined cutoff value; and
ii) determining that the at the subject is at a high risk of having a cell-mediated kidney transplant rejection when the score is greater than or equal to the predetermined cutoff value or determining that the subject is at low risk of having a cell-mediated kidney transplant rejection when the score is less than the predetermined cutoff value.

13. A method of determining the risk of an antibody-mediated kidney transplant rejection in a subject who has undergone a kidney transplant, the method comprising:
a) determining the expression level of at least two of 13 biomarkers in microvesicular RNA isolated from a biological sample from the subject, wherein the 13 biomarkers comprise CD44, NAMPT, PYCARD, IRAK2, IL32, TBP, BCL10, IFNGR1, BMP7, STAT1, ANXA1, TYMP and NFX1;
b) inputting the expression levels from step (a) into an algorithm to generate a score; and
c) determining the risk of an antibody-mediated kidney transplant rejection in the subject based on the score.

14. The method of claim 13, wherein determining the risk of an antibody-mediated kidney transplant rejection in the subject based on the score comprises:
i) comparing the score to a predetermined cutoff value; and
ii) determining that the at the subject is at a high risk of having an antibody mediated. kidney transplant rejection when the score is greater than or equal to the predetermined cutoff value or determining that the subject is at low risk of haying an antibody-mediated kidney transplant rejection when the score is less than the predetermined cutoff value.

15. The method of any one of claims 11-14, wherein step (a) comprises determining the expression level of:
a) at least three of the 13 biomarkers;
b) at least four of the 13 biomarkers;
c) at least five of the 13 biomarkers;
d) at least six of the 13 biomarkers;
e) at least seven of the 13 biomarkers;
f) at least eight of the 13 biomarkers;
g) at least nine of the 13 biomarkers;
h) at least ten of the 13 biomarkers;
i) at least 11 of the 13 biomarkers;
j) at least 12 of the 13 biomarkers.

16. The method of claim 15, wherein step (a) comprises determining the expression level of each of the 13 biomarkers.

17. The method of any of the preceding claims, wherein the biological sample is a urine sample, preferably wherein the urine sample is:
a) a first-catch urine sample: or
b) a second voided urine sample.

18. The method of any of the preceding claims, wherein the biological sample has a volume of between at least about 1 ml to at least about 50 ml, preferably wherein the biological sample has a volume of at least about 3 ml, preferably wherein the biological sample has a volume of up to about 20 ml.

19. The method of any of the preceding claims, wherein step (a) further comprises:
(i) determining the expression level of at least one reference biomarker;
(ii) normalizing the expression level of the at least two biomarkers to the expression level of the at least one reference biomarker, and
wherein inputting the expression levels from step (a) into an algorithm to generate a score comprises inputting the normalized expression levels from step (a) into an algorithm to generate a score.

20. The method of claim 19, wherein the at least one reference biomarker comprises PGK1.

21. The method of any of the preceding claims, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis or any combination thereof.

22. The method of any of the preceding claims, wherein the algorithm is the product of a feature selection wrapper algorithm, a machine learning algorithm, a trained classifier built from at least one predictive classification algorithm or any combination thereof, preferably wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), cforest, classification and regression tree (CART), treebag, k nearest-neighbor (knn), neural network (met), support vector machine-radial (SVM-radial), support vector machine-linear (SVM-linear), naïve bayes (NB), multilayer perceptron (mlp), Boruta or any combination thereof.

23. The method of any of the preceding claims, wherein the predetermined cutoff value has:
i) a negative predictive value of at least about 80%;
ii) a positive predictive value of at least about 80%;
iii) a sensitivity of at least about 80%;
iv) a specificity of at least about 80%; or
v) any combination thereof.

24. The method of any one of the preceding claims, further comprising administering to a subject identified as being at risk for a kidney transplant rejection at least one kidney transplant rejection therapy,
preferably wherein the at least one kidney transplant rejection therapy comprises administering to the subject at least one therapeutically effective amount of at least one immunosuppressant, at least one steroid, at least one corticosteroid, at least one anti-T-cell antibody, mycophenolate mofetil (MMF), cyclosporine A (CsA), tacrolimus, azathioprine, muromonab (OKT-3), anti-thymocyte globulin (ATG), anti-lymphocyte globulin (ALG), Campath (alemtuzurnab), prednisone, mycophenolic acid, rapamycin, belatacept, intravenous immunoglobulin (IVIg), an anti-CD20 agent, rituximab, bortezomib, or any combination thereof.

25. The method of any one of the preceding claims, further comprising administering to a subject identified as being at risk for a cell-mediated kidney transplant rejection at least one cell-mediated kidney transplant rejection therapy,
preferably wherein the at least one cell-mediated kidney transplant rejection therapy comprises administering to the subject at least one therapeutically effective amount of at least one steroid, at least one corticosteroid, muromonab (OKT-3), anti-thymocyte globulin (ATG), Campath (alemtuzumab), prednisone, tacrolimus cyclosporine A, mycophenolic acid, azathioprine, rapamycin, amount of belatacept, or any combination thereof.

26. The method of any one of the preceding claims, further comprising administering to a subject identified as being at risk for an antibody-mediated kidney transplant rejection at least one antibody-mediated kidney transplant rejection therapy,
preferably wherein the at least one antibody-mediated kidney transplant rejection therapy comprises administering to the subject at least one therapeutically effective amount of at least one steroid, at least one corticosteroid, anti-thymocyte globulin (ATG), intravenous immunoglobulin (IVIg), an anti-CD20 agent, rituximab, bortezomib, or any combination thereof.

27. The method of any one of the preceding claims, wherein the subject has not undergone a renal biopsy.
